(19) United States
(12) Patent Application Publication
(10) Pub. No.: US 2026/0314154 A1
(43) Pub. Date: Oct. 8, 2026

(54) PHOTOELECTRIC CONVERSION ELEMENT, IMAGING ELEMENT, OPTICAL SENSOR, AND COMPOUND

(71) Applicant: Fujifilm Corporation, Tokyo (JP)

(72) Inventors: Hiroki Sugiura, Kanagawa (JP); Ryo Fujiwara, Kanagawa (JP); Saika Izumi, Kanagawa (JP); Yosuke Yamamoto, Kanagawa (JP); Yasunori Yonekuta, Kanagawa (JP)

(21) Appl. No.: 19/223,579

(22) Filed: May 30, 2025

Related U.S. Application Data
(63) Continuation of application No. PCT/JP2023/044285, filed on Dec. 11, 2023.

(30) Foreign Application Priority Data
Dec. 20, 2022 (JP) 2022-203403

(51) Int. Cl. H10K 30/30; H10K 30/60; H10K 39/32; H10K 85/20; H10K 85/40; H10K 85/60

(52) U.S. Cl. CPC H10K 30/353; H10K 30/60; H10K 39/32; H10K 85/211; H10K 85/40; H10K 85/622; H10K 85/623; H10K 85/624; H10K 85/626; H10K 85/633; H10K 85/653; H10K 85/654; H10K 85/655; H10K 85/656; H10K 85/6572; H10K 85/6574; H10K 85/6576

(57) ABSTRACT

An object is to provide a photoelectric conversion element having excellent quantum efficiency and having excellent manufacturing suitability. In addition, an object is to provide an imaging element, an optical sensor, and a compound, which are related to the above-described photoelectric conversion element. The photoelectric conversion element of the present invention includes, in the following order, a conductive film, a photoelectric conversion film, and a transparent conductive film, in which the photoelectric conversion film contains a compound represented by Formula (1).

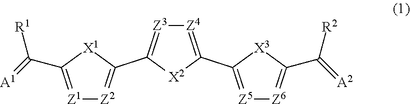

### CROSS-REFERENCE TO RELATED APPLICATIONS

This application is a Continuation of PCT International Application No. PCT/JP2023/044285 filed on Dec. 11, 2023, which claims priority under 35 U.S.C. § 119 (a) to Japanese Patent Application No. 2022-203403 filed on Dec. 20, 2022. The above applications are hereby expressly incorporated by reference, in their entirety, into the present application.

### BACKGROUND OF THE INVENTION

### 1. Field of the Invention

The present invention relates to a photoelectric conversion element, an imaging element, an optical sensor, and a compound.

### 2. Description of the Related Art

In recent years, development of an element (for example, an imaging element) having a photoelectric conversion film has progressed.

For example, JP2019-508376A discloses, as a material used for a photoactive organic electronic component, a compound having a specific structure.

### SUMMARY OF THE INVENTION

With the demand for improvement in performance of an imaging element, an optical sensor, and the like, there is a demand for a photoelectric conversion element which exhibits excellent characteristics. Examples of the characteristics required for the photoelectric conversion element include quantum efficiency. In addition, from the viewpoint of manufacturing efficiency of the element, it is also required to exhibit excellent manufacturing suitability such that the characteristics of the photoelectric conversion element are not deteriorated even in a case where a vapor deposition rate is increased.

Under such a demand, the present inventors have produced a photoelectric conversion element containing the compound disclosed in JP2019-508376A and conducted an examination, and thus found that it is difficult to achieve both the quantum efficiency and the manufacturing suitability.

An object of the present invention is to provide a photoelectric conversion element having excellent quantum efficiency and having excellent manufacturing suitability.

Another object of the present invention is to provide an imaging element, an optical sensor, and a compound, which are related to the above-described photoelectric conversion element.

As a result of conducting an extensive investigation to achieve the objects, the present inventors have found that the objects can be achieved by the following constitution.

[1] A photoelectric conversion element comprising, in the following order:
a conductive film;
a photoelectric conversion film; and
a transparent conductive film,
in which the photoelectric conversion film contains a compound represented by Formula (1) described later.

[2] The photoelectric conversion element according to [1],
in which X¹ is an oxygen atom.

[3] The photoelectric conversion element according to [1] or [2],
in which X¹ and X³ are oxygen atoms.

[4] The photoelectric conversion element according to any one of [1] to [3],
in which the group represented by Formula (A-1) described later is a group represented by Formula (A-2) described later.

[5] The photoelectric conversion element according to any one of [1] to [4],
in which the group represented by Formula (A-1) described later is a group represented by Formula (C-1) described later or a group represented by Formula (C-2) described later.

[6] The photoelectric conversion element according to any one of [1] to [5],
in which R^{Z2} represents a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, which may have a substituent selected from a substituent group S, an acyl group having 2 to 5 carbon atoms, an aromatic ring group which may have a substituent selected from the substituent group S, an aliphatic heterocyclic group which may have a substituent selected from the substituent group S, or a group represented by —Si(R^{Si2})₃,
provided that the linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms, which may have a substituent selected from the substituent group S, each of which is represented by R^{Z2}, may have a halogen atom and may have an etheric oxygen atom, and the acyl group having 2 to 5 carbon atoms, represented by R^{Z2}, may have a halogen atom,
the substituent group S: a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, a halogen atom, and a group represented by —Si(R^{Si2})₃,
the linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms in the substituent group S may have a halogen atom and may have an etheric oxygen atom,
R^{Si2}'s each independently represent a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, which may have a substituent selected from the substituent group S, or an aromatic ring group which may have a substituent selected from the substituent group S, and the linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms, which may have a substituent selected from the substituent group S, each of which is represented by R^{Si2}, may have a halogen atom and may have an etheric oxygen atom.

[7] The photoelectric conversion element according to any one of [1] to [6],
in which R^{Z2} represents a linear aliphatic hydrocarbon group having 1 or 2 carbon atoms, a branched aliphatic hydrocarbon group having 3 or 4 carbon atoms, an acyl group having 2 or 3 carbon atoms, a cyclic aliphatic hydrocarbon group having 3 to 6 carbon atoms, which may have a substituent selected from a substituent group T, an aromatic ring group which may have a substituent selected from the substituent group T, an aliphatic heterocyclic group which may have a substituent selected from the substituent group T, or a group represented by —Si(R^{Si3})₃,
the substituent group T: a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, a halogen atom, and a group represented by —Si(R^{Si3})₃,
R^{Si3}'s each independently represent a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, which may have a substituent selected from the substituent group T, or an aromatic ring group which may have a substituent selected from the substituent group T.

[8] The photoelectric conversion element according to any one of [1] to [7],
in which the photoelectric conversion film further contains an n-type organic semiconductor, and
the photoelectric conversion film has a bulk hetero structure formed in a state in which the compound represented by Formula (1) described later and the n-type organic semiconductor are mixed with each other.

[9] The photoelectric conversion element according to [8],
in which the n-type organic semiconductor includes fullerenes selected from the group consisting of a fullerene and derivatives of the fullerene.

[10] The photoelectric conversion element according to any one of [1] to [9],
in which the photoelectric conversion film further contains a p-type organic semiconductor.

[11] The photoelectric conversion element according to any one of [1] to [10],
in which the photoelectric conversion film further contains a coloring agent.

[12] The photoelectric conversion element according to any one of [1] to [11], further comprising:
one or more interlayers between the conductive film and the transparent conductive film, in addition to the photoelectric conversion film.

[13] An imaging element comprising:
the photoelectric conversion element according to any one of [1] to [12].

[14] An optical sensor comprising:
the photoelectric conversion element according to any one of [1] to [12].

[15] A compound represented by Formula (1) described later.

[16] The compound according to [15],
in which X¹ is an oxygen atom.

[17] The compound according to [15] or [16], in which X¹ and X³ are oxygen atoms.

[18] The compound according to any one of [15] to [17],
in which the group represented by Formula (A-1) described later is a group represented by Formula (A-2) described later.

[19] The compound according to any one of [15] to [18],
in which the group represented by Formula (A-1) described later is a group represented by Formula (C-1) described later or a group represented by Formula (C-2) described later.

[20] The compound according to any one of [15] to [19],
in which R^{Z2} represents a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, which may have a substituent selected from a substituent group S, an acyl group having 2 to 5 carbon atoms, an aromatic ring group which may have a substituent selected from the substituent group S, an aliphatic heterocyclic group which may have a substituent selected from the substituent group S, or a group represented by —Si(R^{Si2})₃,
provided that the linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms, which may have a substituent selected from the substituent group S, each of which is represented by R^{Z2}, may have a halogen atom and may have an etheric oxygen atom, and the acyl group having 2 to 5 carbon atoms, represented by R^{Z2}, may have a halogen atom,
the substituent group S: a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, a halogen atom, and a group represented by —Si(R^{Si2})₃,
the linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms in the substituent group S may have a halogen atom and may have an etheric oxygen atom,
R^{Si2}'s each independently represent a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, which may have a substituent selected from the substituent group S, or an aromatic ring group which may have a substituent selected from the substituent group S, and
the linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms, which may have a substituent selected from the substituent group S, each of which is represented by R^{Si2}, may have a halogen atom and may have an etheric oxygen atom.

[21] The compound according to any one of [15] to [20],
in which R^{Z2} represents a linear aliphatic hydrocarbon group having 1 or 2 carbon atoms, a branched aliphatic hydrocarbon group having 3 or 4 carbon atoms, an acyl group having 2 or 3 carbon atoms, a cyclic aliphatic hydrocarbon group having 3 to 6 carbon atoms, which may have a substituent selected from a substituent group T, an aromatic ring group which may have a substituent selected from the substituent group T, an aliphatic heterocyclic group which may have a substituent selected from the substituent group T, or a group represented by —Si(R^{Si3})₃,
the substituent group T: a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, a halogen atom, and a group represented by —Si(R^{Si3})₃,
R^{Si3}'s each independently represent a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, which may have a substituent selected from the substituent group T, or an aromatic ring group which may have a substituent selected from the substituent group T.

According to the present invention, it is possible to provide a photoelectric conversion element having excellent quantum efficiency and having excellent manufacturing suitability.

In addition, according to the present invention, it is also possible to provide an imaging element, an optical sensor, and a compound, which are related to the above-described photoelectric conversion element.

### BRIEF DESCRIPTION OF THE DRAWINGS

FIG. 1 is a schematic cross-sectional view showing an example of a configuration of a photoelectric conversion element.

FIG. 2 is a schematic cross-sectional view showing an example of a configuration of a photoelectric conversion element.

### DESCRIPTION OF THE PREFERRED EMBODIMENTS

Hereinafter, the present invention will be described in detail.

The description of the configuration requirements described below is made on the basis of representative embodiments of the present invention, but it should not be construed that the present invention is limited to those embodiments.

In the present specification, numerical ranges represented by “to” include numerical values before and after “to” as lower limit values and upper limit values.

In the present specification, a hydrogen atom may be any of a light hydrogen atom (normal hydrogen atom) or a heavy hydrogen atom (for example, a deuterium atom or the like).

In the present specification, in a case of a plurality of substituents, linking groups, and the like (hereinafter, also referred to as “substituent and the like”) represented by specific reference numeral, or in a case of simultaneously defining a plurality of the substituent and the like, it means that each of the substituent and the like may be the same as or different with each other. This also applies to a case of specifying the number of substituents and the like.

In the present specification, the “substituent” includes a group exemplified by the following substituent W, unless otherwise specified.

### (Substituent W)

The substituent W in the present specification will be described below.

Examples of the substituent W include a halogen atom (for example, a fluorine atom, a chlorine atom, a bromine atom, an iodine atom, and the like), an alkyl group (including a cycloalkyl group, a bicycloalkyl group, and a tricycloalkyl group), an alkenyl group (including a cycloalkenyl group and a bicycloalkenyl group), an alkynyl group, an aryl group, a heterocyclic group, a cyano group, a nitro group, an alkoxy group, an aryloxy group, a silyl group, a silyloxy group, a heterocyclic oxy group, an acyloxy group, a carbamoyloxy group, an alkoxycarbonyloxy group, an aryloxycarbonyloxy group, a secondary or tertiary amino group (including an anilino group), an alkylthio group, an arylthio group, a heterocyclic thio group, an alkyl or an arylsulfinyl group, an alkyl or an arylsulfonyl group, an acyl group, an aryloxycarbonyl group, an alkoxycarbonyl group, an aryl or a heterocyclic azo group, an imide group, a phosphino group, a phosphinyl group, a phosphinyloxy group, a phosphinylamino group, a phosphono group, a carboxy group, a phosphoric acid group, a sulfonic acid group, a hydroxy group, a thiol group, an acylamino group, a carbamoyl group, a ureido group, a boronic acid group, and a primary amino group. In addition, each of the above-described groups may further have a substituent (for example, one or more groups of each of the above-described groups), as possible. For example, an alkyl group which may have a substituent is also included as the form of the substituent W.

In addition, in a case where the substituent W has a carbon atom, the number of carbon atoms in the substituent W is, for example, 1 to 20.

The number of atoms other than a hydrogen atom in the substituent W is, for example, 1 to 30.

It is also preferable that a specific compound described later does not contain, as a substituent, a carboxy group, a salt of a carboxy group, a salt of a phosphoric acid group, a sulfonic acid group, a salt of a sulfonic acid group, a hydroxy group, a thiol group, an acylamino group, a carbamoyl group, a ureido group, or a boronic acid group (—B(OH)₂) and/or a primary amino group.

In the present specification, examples of the halogen atom include a fluorine atom, a chlorine atom, a bromine atom, and an iodine atom.

In the present specification, the aliphatic hydrocarbon group may be linear, branched, or cyclic.

Examples of the above-described aliphatic hydrocarbon group include an alkyl group, an alkenyl group, and an alkynyl group.

In addition, in the present specification, unless otherwise specified, the number of carbon atoms in the alkyl group is preferably 1 to 20, more preferably 1 to 10, and still more preferably 1 to 6.

The alkyl group may be linear, branched, or cyclic.

Examples of the alkyl group include a methyl group, an ethyl group, an n-propyl group, an isopropyl group, an n-butyl group, a sec-butyl group, a tert-butyl group, an n-hexyl group, and a cyclopentyl group.

In addition, the alkyl group may be any of a cycloalkyl group, a bicycloalkyl group, or a tricycloalkyl group, and may have a ring structure thereof as a partial structure.

In the alkyl group which may have a substituent, examples of the substituent which may be included in the alkyl group include the groups exemplified as the substituent W. Among these, an aryl group (preferably having 6 to 18 carbon atoms and more preferably having 6 carbon atoms), a heteroaryl group (preferably having 5 to 18 carbon atoms and more preferably having 5 or 6 carbon atoms), or a halogen atom (preferably a fluorine atom or a chlorine atom) is preferable.

In the present specification, unless otherwise specified, an alkyl group moiety in the alkoxy group is preferably the above-described alkyl group. An alkyl group moiety in the alkylthio group is preferably the above-described alkyl group.

In the alkoxy group which may have a substituent, examples of the substituent which may be included in the alkoxy group include the same examples as the substituent in the alkyl group which may have a substituent.

In the alkylthio group which may have a substituent, examples of the substituent which may be included in the alkylthio group include the same examples as the substituent in the alkyl group which may have a substituent.

In the present specification, the alkenyl group may be any of linear, branched, or cyclic, unless otherwise specified. The number of carbon atoms in the above-described alkenyl group is preferably 2 to 20. In the alkenyl group which may have a substituent, examples of the substituent which may be included in the alkenyl group include the same examples as the substituent in the alkyl group which may have a substituent.

In the present specification, the alkynyl group may be any of linear, branched, or cyclic, unless otherwise specified. The number of carbon atoms in the above-described alkynyl group is preferably 2 to 20. In the alkynyl group which may have a substituent, examples of the substituent which may be included in the alkynyl group include the same examples as the substituent in the alkyl group which may have a substituent.

In the present specification, unless otherwise specified, an aromatic ring or an aromatic ring constituting the aromatic ring group may be any of a monocyclic ring or a polycyclic ring (for example, 2 to 6 rings or the like). The monocyclic aromatic ring is an aromatic ring having only one aromatic ring structure as a ring structure. The polycyclic (for example, 2 to 6 rings or the like) aromatic ring is an aromatic ring formed by a plurality of (for example, 2 to 6 or the like) fused aromatic ring structures, as a ring structure.

The number of ring members in the above-described aromatic ring is preferably 5 to 15.

The above-described aromatic ring may be an aromatic hydrocarbon ring or an aromatic heterocyclic ring.

In a case where the above-described aromatic ring is an aromatic heterocyclic ring, the number of heteroatoms included as ring member atoms is, for example, 1 to 10. Examples of the above-described heteroatom include a nitrogen atom, a sulfur atom, an oxygen atom, a selenium atom, a tellurium atom, a phosphorus atom, a silicon atom, and a boron atom.

Examples of the above-described aromatic hydrocarbon ring include a benzene ring, a naphthalene ring, an anthracene ring, a phenanthrene ring, and a fluorene ring.

Examples of the above-described aromatic heterocyclic ring include a pyridine ring, a pyrimidine ring, a pyridazine ring, a pyrazine ring, a triazine ring (for example, 1,2,3-triazine ring, 1,2,4-triazine ring, 1,3,5-triazine ring, and the like), a tetrazine ring (for example, 1,2,4,5-tetrazine ring and the like), a quinoxaline ring, a pyrrole ring, a furan ring, a thiophene ring, an imidazole ring, an oxazole ring, a thiazole ring, a benzopyrrole ring, a benzofuran ring, a benzothiophene ring, a benzimidazole ring, a benzoxazole ring, a benzothiazole ring, a naphthopyrrole ring, a naphthofuran ring, a naphthothiophene ring, a naphthimidazole ring, a naphthoxazole ring, a pyrroloimidazole ring (for example, a 5H-pyrrolo[1,2-a]imidazole ring and the like), an imidazooxazole ring (for example, an imidazo[2,1-b]oxazole ring and the like), a thienothiazole ring (for example, a thieno[2,3-d]thiazole ring and the like), a benzothiadiazole ring, a benzodithiophene ring (for example, benzo[1,2-b: 4,5-b′]dithiophene ring and the like), a thienothiophene ring (for example, thieno[3,2-b]thiophene ring and the like), a thiazolothiazole ring (for example, thiazolo[5,4-d]thiazole ring and the like), a naphthodithiophene ring (for example, a naphtho[2,3-b: 6,7-b′]dithiophene ring, a naphtho[2,1-b: 6,5-b′]dithiophene ring, a naphtho[1,2-b: 5,6-b′]dithiophene ring, a 1,8-dithiadicyclopenta[b,g]naphthalene ring, and the like), a benzothienobenzothiophene ring, a dithieno[3,2-b: 2′,3′-d]thiophene ring, and a 3,4,7,8-tetrathiadicyclopenta[a,e]pentalene ring.

In the aromatic ring which may have a substituent, examples of the type of the substituent which may be included in the aromatic ring include the groups exemplified as the substituent W. In a case where the above-described aromatic ring has a substituent, the number of substituents may be 1 or more (for example, 1 to 4 or the like).

In the present specification, examples of the “aromatic ring group” include a group obtained by removing one or more (for example, 1 to 5 or the like) hydrogen atoms from the above-described aromatic ring.

In the present specification, examples of the “aryl group” include a group obtained by removing one hydrogen atom from a ring corresponding to the aromatic hydrocarbon ring among the above-described aromatic rings.

In the present specification, examples of the “heteroaryl group” include a group obtained by removing one hydrogen atom from a ring corresponding to the aromatic heterocyclic ring among the above-described aromatic rings.

In the present specification, examples of the “arylene group” include a group obtained by removing two hydrogen atoms from a ring corresponding to the aromatic hydrocarbon ring among the above-described aromatic rings.

In the present specification, examples of the “heteroarylene group” include a group obtained by removing two hydrogen atoms from a ring corresponding to the aromatic heterocyclic ring among the above-described aromatic rings.

In the aromatic ring group which may have a substituent, the aryl group which may have a substituent, the heteroaryl group which may have a substituent, the arylene group which may have a substituent, and the heteroarylene group which may have a substituent, examples of the type of the substituent which may be included in these groups include the groups exemplified as the substituent W. In a case where these groups each of which may have a substituent have a substituent, the number of substituents may be 1 or more (for example, 1 to 4 or the like).

In the present specification, the non-aromatic ring refers to a ring which does not correspond to the aromatic ring, and examples thereof include an aliphatic hydrocarbon ring and an aliphatic heterocyclic ring.

Examples of the above-described aliphatic hydrocarbon ring include a cycloalkane, a cycloalkene, and a cycloalkyne.

Examples of the above-described aliphatic heterocyclic ring include a pyrrolidine ring, an oxolane ring, a thiolane ring, a piperidine ring, a tetrahydropyran ring, a thiacine ring, a piperazine ring, a morpholine ring, a quinocyclidine ring, an azetidine ring, an oxetane ring, an aziridine ring, a dioxane ring, and a γ-butyrolactone ring.

In the present specification, examples of the “aliphatic hydrocarbon ring group” include a group obtained by removing one or more (for example, 1 to 5 or the like) hydrogen atoms from a ring corresponding to the aliphatic hydrocarbon ring.

In the present specification, examples of the “aliphatic heterocyclic group” include a group obtained by removing one or more (for example, 1 to 5 or the like) hydrogen atoms from a ring corresponding to the aliphatic heterocyclic ring.

In the present specification, in a case where a plurality of identical symbols indicating a type or the number of groups are present in the formula, which indicates a chemical structure, contents of these plurality of identical symbols indicating a type or the number of groups are independent of each other, and the contents of the identical symbols may be the same or different from each other, unless otherwise specified.

In the present specification, in a case where a plurality of identical groups (for example, alkyl groups and the like) are present in the formula, which indicates a chemical structure, specific contents between these plurality of identical groups are independent of each other, and the specific contents between the plurality of identical groups may be the same or different from each other, unless otherwise specified.

A bonding direction of a divalent group (for example, —CO—O—) described in the present specification is not limited unless otherwise specified. For example, in a case where Y in a compound represented by a formula “X—Y—Z” is —CO—O—, the compound may be any of “X—O—CO—Z” or “X—CO—O—Z”.

In the present specification, regarding a compound which may have a geometric isomer (cis-trans isomer), a general formula or a structural formula representing the compound may be described only in the form of either a cis isomer or a trans isomer for convenience. Even in such a case, unless otherwise specified, the form of the compound is not limited to either the cis isomer or the trans isomer, and the compound may be the cis isomer or the trans isomer.

In the present specification, unless otherwise specified, * in the formula represents a bonding position.

### [Photoelectric Conversion Element]

The photoelectric conversion element according to the embodiment of the present invention includes a conductive film, a photoelectric conversion film, and a transparent conductive film in this order, in which the photoelectric conversion film contains a compound represented by Formula (1) described later (hereinafter, referred to as “specific compound”).

The reason why the photoelectric conversion film having the above-described configuration can achieve the object of the present invention is not necessarily clear, but the present inventors speculate as follows.

The mechanism by which the effect is obtained is not limited by the following supposition. In other words, even in a case where an effect is obtained by a mechanism other than the following, it is included in the scope of the present invention.

Since the specific compound has one or more specific substituents represented by R^{Z2} at a donor site, and at least one of X¹, X², or X³ is an oxygen atom, intermolecular interaction of the specific compound is appropriately controlled. Accordingly, excessive aggregation between the specific compounds is suppressed, charge separation in the photoelectric conversion film proceeds efficiently, and as a result, the quantum efficiency of the photoelectric conversion element is excellent.

Furthermore, since an acceptor site of the specific compound has a specific ring structure, the donor site has a specific substituent represented by R^{Z2}, and at least one of X¹, X², or X³ is an oxygen atom, heat resistance and sublimation temperature of the specific compound are appropriately controlled. Accordingly, even in a case where a vapor deposition rate is increased, decomposition of the specific compound is suppressed, and as a result, the manufacturing suitability is also excellent.

Hereinafter, the fact that at least one of the quantum efficiency or the manufacturing suitability of the photoelectric conversion element is more excellent is also referred to as “effect of the present invention is more excellent”.

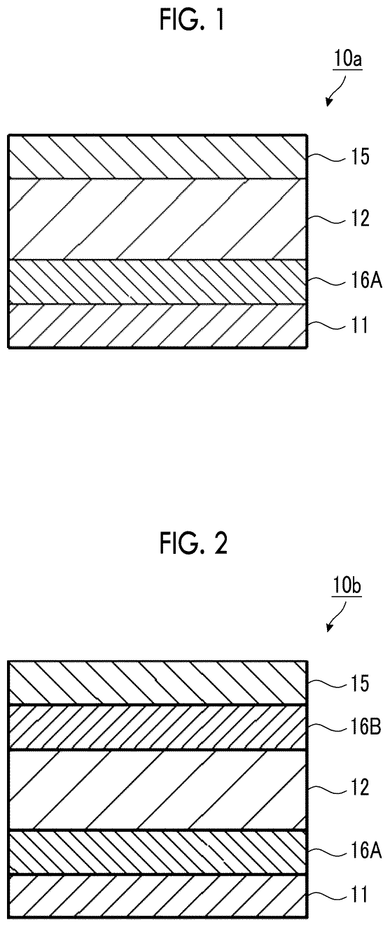

FIG. 1 shows a schematic cross-sectional view of one embodiment of the photoelectric conversion element according to the present invention.

A photoelectric conversion element 10a shown in FIG. 1 has a configuration in which a conductive film (hereinafter, also referred to as “lower electrode”) 11 functioning as a lower electrode, an electron blocking film 16A, a photoelectric conversion film 12 containing the specific compound, and a transparent conductive film (hereinafter, also referred to as “upper electrode”) 15 functioning as an upper electrode are laminated in this order.

FIG. 2 shows a configuration example of another photoelectric conversion element. A photoelectric conversion element 10b shown in FIG. 2 has a configuration in which the electron blocking film 16A, the photoelectric conversion film 12, a hole blocking film 16B, and the upper electrode 15 are laminated on the lower electrode 11 in this order. The lamination order of the electron blocking film 16A, the photoelectric conversion film 12, and the hole blocking film 16B in FIGS. 1 and 2 may be appropriately changed according to the application and the characteristics.

In the photoelectric conversion element 10a (or 10b), it is preferable that light is incident to the photoelectric conversion film 12 through the upper electrode 15.

In a case where the photoelectric conversion element 10a (or 10b) is used, a voltage can be applied. In this case, it is preferable that the lower electrode 11 and the upper electrode 15 form a pair of electrodes, and a voltage of 1×10⁻⁵ to 1×10⁷ V/cm is applied between the pair of electrodes. From the viewpoint of performance and power consumption, the applied voltage is more preferably 1×10⁻⁴ to 1×10⁷ V/cm and still more preferably 1×10⁻³ to 5×10⁶ V/cm.

Regarding the voltage application method, in FIGS. 1 and 2, it is preferable that the voltage is applied such that the electron blocking film 16A side is a cathode and the photoelectric conversion film 12 side is an anode. In a case where the photoelectric conversion element 10a (or 10b) is used as an optical sensor, or also in a case of being incorporated in an imaging element, the voltage can be applied by the same method.

As described in detail below, the photoelectric conversion element 10a (or 10b) can be suitably applied to applications of an imaging element.

Hereinafter, the form of each layer constituting the photoelectric conversion element according to the embodiment of the present invention will be described in detail.

### [Photoelectric Conversion Film]

The photoelectric conversion element includes a photoelectric conversion film.

### <Specific Compound>

The photoelectric conversion film contains the specific compound which is a compound represented by Formula (1).

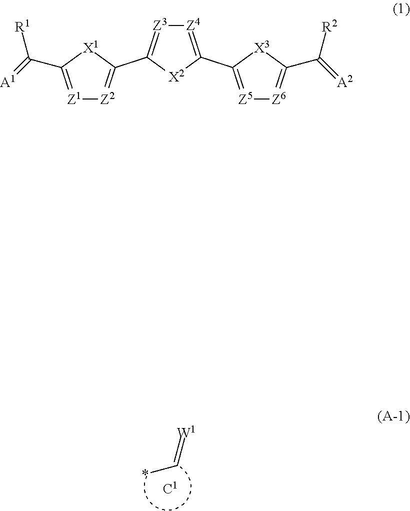

In Formula (1), R¹ and R² each independently represent a hydrogen atom or a substituent.

X¹ to X³ each independently represent a sulfur atom, an oxygen atom, a selenium atom, or a tellurium atom. However, at least one of X¹, X², or X³ is an oxygen atom.

Z¹ to Z⁶ each independently represent —CR^{Z1}═ or —N═. R^{Z1} represents a hydrogen atom or a substituent. However, at least one of Z¹, . . . , or Z⁶ is —CR^{Z2}═. R^{Z2} represents an aliphatic hydrocarbon group which may have a substituent, an acyl group which may have a substituent, an aromatic ring group which may have a substituent, an aliphatic heterocyclic group which may have a substituent, or a group represented by —Si(R^{Si})₃.

The aliphatic hydrocarbon group represented by R^{Z2} may have a halogen atom and may have an etheric oxygen atom. The acyl group represented by R^{Z2} may have a halogen atom.

R^{Si}'s each independently represent an aliphatic hydrocarbon group which may have a substituent or an aromatic ring group which may have a substituent.

The aliphatic hydrocarbon group represented by R^{Si} may have a halogen atom and may have an etheric oxygen atom.

In a case where Z¹ and Z² are —CR^{Z2}═, two R^{Z2}'s may be bonded to each other to form a ring; in a case where Z³ and Z⁴ are —CR^{Z2}═, two R^{Z2}'s may be bonded to each other to form a ring; and in a case where Z⁵ and Z⁶ are —CR^{Z2}═, two R^{Z2}'s may be bonded to each other to form a ring.

A¹ and A² each independently represent a group represented by Formula (A-1).

In Formula (A-1), C¹ represents a ring which contains at least two carbon atoms and may have a substituent.

W¹ represents an oxygen atom, a sulfur atom, ═NR^{W1}, or ═CR^{W2}R^{W3}. R^{W1} represents a hydrogen atom or a substituent. R^{W2} and R^{W3} each independently represent a cyano group, —SO₂R^{W4}, —COOR^{W5}, or —COR^{W6}. R^{W4}, R^{W5}, and R^{W6} each independently represent an aliphatic hydrocarbon group which may have a substituent, an aromatic ring group which may have a substituent, or an aliphatic heterocyclic group which may have a substituent.

In Formula (1), R¹ and R² each independently represent a hydrogen atom or a substituent.

Examples of the above-described substituent include the substituents exemplified as the substituent W.

From the viewpoint that the effect of the present invention is more excellent, R¹ and R² are preferably hydrogen atoms.

In Formula (1), X¹ to X³ each independently represent a sulfur atom, an oxygen atom, a selenium atom, or a tellurium atom. However, at least one of X¹, X², or X³ is an oxygen atom.

From the viewpoint that the effect of the present invention is more excellent, X¹ to X³ are preferably a sulfur atom or an oxygen atom, and more preferably an oxygen atom.

At least one of X¹, X², or X³ is an oxygen atom, and it is preferable that two or more thereof are oxygen atoms.

Among these, from the viewpoint that the effect of the present invention is more excellent, it is preferable that at least one of X¹ or X³ is an oxygen atom, and it is more preferable that X¹ and X³ are oxygen atoms.

The compound represented by Formula (1) is preferably a compound represented by Formula (1-1) to Formula (1-5); more preferably a compound represented by Formula (1-1) to Formula (1-4); still more preferably a compound represented by Formula (1-1) or Formula (1-4); and particularly preferably a compound represented by Formula (1-1).

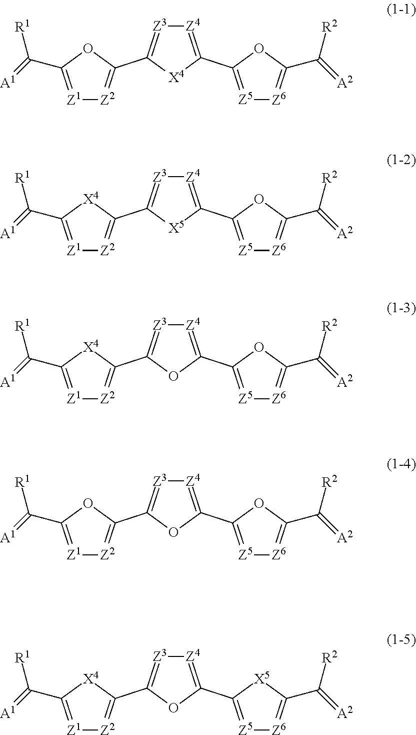

In Formula (1-1) to Formula (1-5), R¹, R², Z¹ to Z⁶, A¹, and A² have the same meaning as R¹, R², Z¹ to Z⁶, A¹, and A² in Formula (1).

X⁴ and X⁵ each independently represent a sulfur atom, a selenium atom, or a tellurium atom.

X⁴ and X⁵ are preferably sulfur atoms.

In Formula (1), Z¹ to Z⁶ each independently represent —CR^{Z1}═ or —N═.

R^{Z1} represents a hydrogen atom or a substituent.

Examples of the substituent represented by R^{Z1} include the substituents exemplified as the substituent W.

From the viewpoint that the effect of the present invention is more excellent, R^{Z1} is preferably a hydrogen atom, a halogen atom, or a group represented by R^{Z2}, which will be described later.

In a case where a plurality of R^{Z1}'s are present, groups represented by the plurality of R^{Z1}'s may be the same or different from each other.

At least one of Z¹, . . . , or Z⁶ is —CR^{Z1}═; and it is preferable that at least three thereof are —CR^{Z1}═, and it is more preferable that at least five thereof are —CR^{Z1}═.

In Formula (1), at least one of Z¹, . . . , or Z⁶ is —CR^{Z2}═.

It is preferable that one to four of Z¹ to Z⁶ are —CR^{Z2}═.

In a case where a plurality of R^{Z2}'s are present, groups represented by the plurality of R^{Z2}'s may be the same or different from each other.

In a case where Z¹ and Z² are —CR^{Z2}═, two R^{Z2}'s may be bonded to each other to form a ring; in a case where Z³ and Z⁴ are —CR^{Z2}═, two R^{Z2}'s may be bonded to each other to form a ring; and in a case where Z⁵ and Z⁶ are —CR^{Z2}═, two R^{Z2}'s may be bonded to each other to form a ring.

Examples of the ring formed by bonding two R^{Z2}'s to each other include an aliphatic hydrocarbon ring and an aliphatic heterocyclic ring.

The number of ring members in the above-described ring is not particularly limited, but is preferably 3 to 12, more preferably 4 to 6, and still more preferably 5.

Examples of a heteroatom which is included in the above-described aliphatic heterocyclic ring include a sulfur atom, an oxygen atom, a nitrogen atom, a selenium atom, a tellurium atom, a phosphorus atom, a silicon atom, and a boron atom; and a sulfur atom, an oxygen atom, or a nitrogen atom is preferable.

In Formula (1), R^{Z2} represents an aliphatic hydrocarbon group which may have a substituent, an acyl group which may have a substituent, an aromatic ring group which may have a substituent, an aliphatic heterocyclic group which may have a substituent, or a group by —Si(R^{Si})₃.

The above-described aliphatic hydrocarbon group represented by R^{Z2} may have a halogen atom and may have an etheric oxygen atom. The above-described acyl group represented by R^{Z2} may have a halogen atom.

In the present specification, the fact that the aliphatic hydrocarbon group may have an etheric oxygen atom means that the aliphatic hydrocarbon group may have a divalent linking group represented by —O— in or at a terminal of the aliphatic hydrocarbon group.

Examples of the above-described aliphatic hydrocarbon group represented by R^{Z2} include a linear aliphatic hydrocarbon group, a branched aliphatic hydrocarbon group, and a cyclic aliphatic hydrocarbon group.

The number of carbon atoms in the above-described linear aliphatic hydrocarbon group is preferably 1 to 20, more preferably 1 to 6, still more preferably 1 to 3, and particularly preferably 1 or 2. Specific examples thereof include a methyl group, an ethyl group, an n-propyl group, an n-butyl group, an n-pentyl group, an n-hexyl group, an n-heptyl group, an n-octyl group, an n-nonyl group, an n-decyl group, an n-undecyl group, an n-dodecyl group, a vinyl group, an allyl group, an ethynyl group, and a propargyl group; and a methyl group, an ethyl group, an n-propyl group, a vinyl group, an ethynyl group, or a propargyl group is preferable, a methyl group, an ethyl group, or an ethynyl group is more preferable, and a methyl group or an ethyl group is still more preferable.

The number of carbon atoms in the above-described branched aliphatic hydrocarbon group is preferably 3 to 20, more preferably 3 to 10, still more preferably 3 to 6, and particularly preferably 3 or 4. Specific examples thereof include an isopropyl group, a sec-butyl group, an iso-butyl group, a tert-butyl group, a neopentyl group, a 2-ethylhexyl group, a 3,7-dimethyloctyl group, a 2-butyloctyl group, a 2-hexyloctyl group, a 2-hexyldodecyl group, and a 2-octyldodecyl group; and an isopropyl group or a tert-butyl group is preferable.

The above-described cyclic aliphatic hydrocarbon group may be monocyclic or polycyclic.

The number of carbon atoms in the above-described cyclic aliphatic hydrocarbon group is preferably 3 to 10, more preferably 3 to 8, and still more preferably 3 to 6. Specific examples thereof include a cyclopropyl group, a cyclobutyl group, a cyclopentyl group, a cyclohexyl group, a cycloheptyl group, a cyclooctyl group, a cyclodecanyl group, a dicyclobutanyl group, a bicyclo[1.1.1]pentyl group, and a bicyclo[2.2.2]pentyl group; and a cyclopropyl group, a cyclobutyl group, a cyclopentyl group, or a cyclohexyl group is preferable, and a cyclopropyl group is more preferable.

The above-described aliphatic hydrocarbon group may have a halogen atom. Examples of the halogen atom which may be included in the above-described aliphatic hydrocarbon group include a fluorine atom, a chlorine atom, a bromine atom, and an iodine atom; and a fluorine atom or a chlorine atom is preferable.

The above-described aliphatic hydrocarbon group may have an etheric oxygen atom. Examples of the aliphatic hydrocarbon group having an etheric oxygen atom include a methoxy group, an ethoxy group, an isopropoxy group, a cyclopropoxy group, and a methoxyethyl group.

Examples of the substituent which may be included in the above-described aliphatic hydrocarbon group include the substituents exemplified as the substituent W; and a substituent selected from a substituent group S described later is preferable, and a substituent selected from a substituent group T described later is more preferable.

A hydrocarbon group contained in the above-described acyl group which may have a substituent, represented by R^{Z2}, may be an aliphatic hydrocarbon group or an aromatic hydrocarbon group, and is preferably an aliphatic hydrocarbon group.

The aliphatic hydrocarbon group included in the above-described acyl group may be linear, branched, or cyclic.

The number of carbon atoms in the aliphatic hydrocarbon group included in the above-described acyl group is preferably 1 to 20, more preferably 1 to 10, still more preferably 1 to 4, and particularly preferably 1 or 2. Specific examples thereof include a linear aliphatic hydrocarbon group such as a methyl group, an ethyl group, an n-propyl group, an n-butyl group, an n-pentyl group, an n-hexyl group, an n-heptyl group, an n-octyl group, an n-nonyl group, an n-decyl group, an n-undecyl group, and an n-dodecyl group, a branched aliphatic hydrocarbon group such as an isopropyl group, a sec-butyl group, an iso-butyl group, a tert-butyl group, a neopentyl group, a 1-ethylpentyl group, a 2,6-dimethylheptyl group, a 1-butylheptyl group, a 1-hexylheptyl group, a 1-hexylundecyl group, and a 1-octylundecyl group, and a cyclic aliphatic hydrocarbon group such as a cyclopropyl group, a cyclobutyl group, a cyclopentyl group, a cyclohexyl group, a cycloheptyl group, a cyclooctyl group, a cyclodecanyl group, a dicyclobutanyl group, a bicyclo[1.1.1]pentyl group, and a bicyclo[2.2.2]pentyl group; and a methyl group, an ethyl group, an n-propyl group, an isopropyl group, or a tert-butyl group is preferable, and a methyl group or an ethyl group is more preferable.

The number of carbon atoms in the aromatic hydrocarbon group included in the above-described acyl group is preferably 6 to 20, more preferably 6 to 10, and still more preferably 6. Specific examples thereof include a phenyl group, a naphthyl group, an anthryl group, a pyrenyl group, a phenanthrenyl group, and a fluorenyl group; and a phenyl group is preferable.

The number of carbon atoms in the above-described acyl group is preferably 2 to 21, more preferably 2 to 11, still more preferably 2 to 5, and particularly preferably 2 or 3.

Examples of the above-described acyl group include an acetyl group, a propionyl group, a butyryl group, an isobutyryl group, a pivaloyl group, a hexanoyl group, and a benzoyl group; and an acetyl group or a propionyl group is preferable.

The above-described acyl group may have a halogen atom. Examples of the halogen atom which may be included in the above-described acyl group include a fluorine atom, a chlorine atom, a bromine atom, and an iodine atom; and a fluorine atom or a chlorine atom is preferable.

Examples of the substituent which may be included in the above-described acyl group include the substituents exemplified as the substituent W; and a substituent selected from the substituent group S described later is preferable, and a substituent selected from the substituent group T described later is more preferable.

The above-described aromatic ring group which may have a substituent, represented by R^{Z2}, may be monocyclic or polycyclic.

The above-described aromatic ring group may be an aromatic hydrocarbon group or an aromatic heterocyclic group, and an aromatic hydrocarbon group is preferable.

The number of ring members in the above-described aromatic ring group is preferably 5 to 20, more preferably 5 to 12, and still more preferably 5 to 8.

The number of carbon atoms in the above-described aromatic ring group is preferably 30 or less, more preferably 20 or less, and still more preferably 10 or less. The lower limit thereof is preferably 1 or more, more preferably 3 or more, and still more preferably 4 or more.

Examples of a heteroatom which is included in the above-described aromatic heterocyclic group include a sulfur atom, an oxygen atom, a nitrogen atom, a selenium atom, a tellurium atom, a phosphorus atom, a silicon atom, and a boron atom; and a sulfur atom, an oxygen atom, or a nitrogen atom is preferable.

Examples of the above-described aromatic ring group include an aromatic hydrocarbon ring group such as a phenyl group, a naphthyl group, an anthryl group, a pyrenyl group, a phenanthrenyl group, and a fluorenyl group, and an aromatic heterocyclic group such as a pyridine ring group, a pyrimidine ring group, a pyridazine ring group, a pyrazine ring group, a triazine ring group, a tetrazine ring group, a quinoxaline ring group, a pyrrole ring group, a furan ring group, a thiophene ring group, an imidazole ring group, an oxazole ring group, a pyrazole ring group, a thiazole ring group, a benzopyrrole ring group, a benzofuran ring group, a benzothiophene ring group, a benzimidazole ring group, a benzoxazole ring group, and a benzothiazole ring group; and a phenyl group, a thiophene ring group, a furan ring group, or a pyridine ring group is preferable, a phenyl group or a thiophene ring group is more preferable, and a phenyl group is still more preferable.

Examples of the substituent which may be included in the above-described aromatic ring group include the substituents exemplified as the substituent W; and a substituent selected from the substituent group S described later is preferable, and a substituent selected from the substituent group T described later is more preferable.

In a case where the above-described aromatic ring group has a substituent, the number of substituents is not particularly limited, but is preferably 1 to 6 and more preferably 1 to 3.

The above-described aliphatic heterocyclic group which may have a substituent, represented by R^{Z2}, may be monocyclic or polycyclic.

The number of ring members in the above-described aliphatic heterocyclic group is preferably 6 to 20, more preferably 6 to 12, and still more preferably 6 to 8.

The number of carbon atoms in the above-described aliphatic heterocyclic group is preferably 1 to 30, more preferably 3 to 20, and still more preferably 4 to 10.

Examples of a heteroatom which is included in the above-described aliphatic heterocyclic group include a sulfur atom, an oxygen atom, a nitrogen atom, a selenium atom, a tellurium atom, a phosphorus atom, a silicon atom, and a boron atom; and a sulfur atom, an oxygen atom, or a nitrogen atom is preferable.

Examples of the above-described aliphatic heterocyclic group include a pyrrolidine ring group, an oxolane ring group, a thiolane ring group, a piperidine ring group, a tetrahydrofuran ring group, a tetrahydropyran ring group, a thiane ring group, a piperazine ring group, a morpholine ring group, a quinocyclidine ring group, an azetidine ring group, an oxetane ring group, an aziridine ring group, a dioxane ring group, a pentamethylenesulfide ring group, and a γ-butyrolactone ring group; and a piperidine ring group is preferable.

Examples of the substituent which may be included in the above-described aliphatic heterocyclic group include the substituents exemplified as the substituent W; and a substituent selected from the substituent group S described later is preferable, and a substituent selected from the substituent group T described later is more preferable.

In a case where the above-described aliphatic heterocyclic group has a substituent, the number of substituents is not particularly limited, but is preferably 1 to 4 and more preferably 1 to 3.

In the group represented by —Si(R^{Si})₃, R^{Si}'s each independently represent an aliphatic hydrocarbon group which may have a substituent or an aromatic ring group which may have a substituent.

The above-described aliphatic hydrocarbon group which may have a substituent, represented by R^{Si}, may have a halogen atom and may have an etheric oxygen atom.

Definition and suitable aspects of the aliphatic hydrocarbon group which may have a substituent, represented by R^{Si}, are the same as those of the aliphatic hydrocarbon group which may have a substituent, represented by R^{Z2}; and definition and suitable aspects of the aromatic ring group which may have a substituent, represented by R^{Si}, are the same as those of the aromatic ring group which may have a substituent, represented by R^{Z2}.

Among these, the group represented by —Si(R^{Si})₃ is preferably a group represented by —Si(R^{Si2})₃. R^{Si2}'s each independently represent a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, which may have a substituent selected from the substituent group S described later, or an aromatic ring group which may have a substituent selected from the substituent group S described later.

The linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms, which may have a substituent selected from the substituent group S, each of which is represented by R^{Si2}, may have a halogen atom and may have an etheric oxygen atom.

The group represented by —Si(R^{Si})₃ is more preferably a group represented by —Si(R^{Si3})₃. R^{Si3}'s each independently represent a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, which may have a substituent selected from the substituent group T described later, or an aromatic ring group which may have a substituent selected from the substituent group T described later.

Specific examples of the group represented by —Si(R^{Si})₃ include a trimethylsilyl group, a triethylsilyl group, a dimethylisopropylsilyl group, a diethylisopropylsilyl group, a cyclohexyldimethylsilyl group, a dimethylphenylsilyl group, and a tert-butyldimethylsilyl group; and a trimethylsilyl group or a triethylsilyl group is preferable, and a trimethylsilyl group is more preferable.

From the viewpoint that the effect of the present invention is more excellent, R^{Z2} is preferably a group selected from a substituent group R1.

Substituent group R1: a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, which may have a substituent selected from the substituent group S described later, an acyl group having 2 to 5 carbon atoms, an aromatic ring group which may have a substituent selected from the substituent group S described later, an aliphatic heterocyclic group which may have a substituent selected from the substituent group S described later, and the group represented by —Si(R^{Si2})₃.

In the above-described substituent group R1, the linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms, which may have a substituent selected from the substituent group S, may have a halogen atom and may have an etheric oxygen atom.

From the viewpoint that the effect of the present invention is more excellent, R^{Z2} is more preferably a group selected from a substituent group R².

Substituent group R²: a linear aliphatic hydrocarbon group having 1 or 2 carbon atoms, a branched aliphatic hydrocarbon group having 3 or 4 carbon atoms, an acyl group having 2 or 3 carbon atoms, a cyclic aliphatic hydrocarbon group having 3 to 6 carbon atoms, which may have a substituent selected from the substituent group T described later, an aromatic ring group which may have a substituent selected from the substituent group T described later, an aliphatic heterocyclic group which may have a substituent selected from the substituent group T described later, and the group represented by —Si(R^{Si3})₃.

Hereinafter, the substituent group S and substituent group T will be described in detail.

Substituent group S: a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, a halogen atom, and a group represented by —Si(R^{Si2})₃.

Specific examples of the above-described linear aliphatic hydrocarbon group having 1 to 3 carbon atoms in the above-described substituent group S include a methyl group, an ethyl group, an n-propyl group, a vinyl group, an allyl group, an ethynyl group, and a propargyl group; and a methyl group or an ethyl group is preferable, and a methyl group is more preferable.

Specific examples of the above-described branched-chain aliphatic hydrocarbon group having 3 or 4 carbon atoms in the above-described substituent group S include an isopropyl group, a sec-butyl group, an iso-butyl group, and a tert-butyl group; and an isopropyl group or a tert-butyl group is preferable, and an isopropyl group is more preferable.

The above-described cyclic aliphatic hydrocarbon group having 3 to 8 carbon atoms in the above-described substituent group S may be monocyclic or polycyclic, and is preferably monocyclic. Specific examples of the above-described cyclic aliphatic hydrocarbon group having 3 to 8 carbon atoms include a cyclopropyl group, a cyclobutyl group, a cyclopentyl group, a cyclohexyl group, a cycloheptyl group, and a cyclooctyl group; and a cyclopropyl group, a cyclobutyl group, a cyclopentyl group, or a cyclohexyl group is preferable, and a cyclopropyl group is more preferable.

The linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms in the above-described substituent group S (hereinafter, also referred to as “aliphatic hydrocarbon group in the substituent group S”) may have a halogen atom.

Examples of the halogen atom which may be included in the above-described aliphatic hydrocarbon group in the substituent group S include a fluorine atom, a chlorine atom, a bromine atom, and an iodine atom; and a fluorine atom or a chlorine atom is preferable.

The above-described aliphatic hydrocarbon group in the substituent group S may have an etheric oxygen atom. Examples of the aliphatic hydrocarbon group having an etheric oxygen atom in the substituent group S include a methoxy group, an ethoxy group, a methoxyethyl group, an isopropoxy group, and a cyclopropoxy group; and a methoxy group is preferable.

Examples of the halogen atom in the above-described substituent group S include a fluorine atom, a chlorine atom, a bromine atom, and an iodine atom; and a fluorine atom or a chlorine atom is preferable.

In the group represented by —Si(R^{Si2})₃ of the above-described substituent group S, R^{Si2}'s each independently represent a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, which may have a substituent selected from the substituent group S, or an aromatic ring group which may have a substituent selected from the substituent group S.

The linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms, which may have a substituent selected from the substituent group S, each of which is represented by R^{Si2}, may have a halogen atom and may have an etheric oxygen atom.

Definition and suitable aspects of the group represented by —Si(R^{Si2})₃ of the above-described substituent group S are the same as those of the group represented by —Si(R^{Si2})₃ in R^{Z2}.

From the viewpoint that the effect of the present invention is more excellent, the substituent selected from the substituent group S is preferably a substituent selected from the substituent group T.

Substituent group T: a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, a halogen atom, and a group represented by —Si(R^{Si3})₃.

Specific examples of the above-described linear aliphatic hydrocarbon group having 1 or 2 carbon atoms in the above-described substituent group T include a methyl group and an ethyl group; and a methyl group is preferable.

Specific examples of the above-described branched-chain aliphatic hydrocarbon group having 3 or 4 carbon atoms in the above-described substituent group T include an isopropyl group, a sec-butyl group, an iso-butyl group, and a tert-butyl group; and an isopropyl group or a tert-butyl group is preferable, and an isopropyl group is more preferable.

The above-described cyclic aliphatic hydrocarbon group having 3 to 6 carbon atoms in the above-described substituent group T may be monocyclic or polycyclic, and is preferably monocyclic. Specific examples of the above-described cyclic aliphatic hydrocarbon group having 3 to 6 carbon atoms include a cyclopropyl group, a cyclobutyl group, a cyclopentyl group, and a cyclohexyl group; and a cyclopropyl group is preferable.

Examples of the halogen atom in the above-described substituent group T include a fluorine atom, a chlorine atom, a bromine atom, and an iodine atom; and a fluorine atom or a chlorine atom is preferable.

In the group represented by —Si(R^{Si3})₃ of the above-described substituent group T, R^{Si3}'s each independently represent a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, which may have a substituent selected from the substituent group T, or an aromatic ring group which may have a substituent selected from the substituent group T.

Definition and suitable aspects of the group represented by —Si(R^{Si3})₃ of the above-described substituent group T are the same as those of the group represented by —Si(R^{Si3})₃ in R^{Z2}.

In Formula (1), A¹ and A² each independently represent a group represented by Formula (A-1).

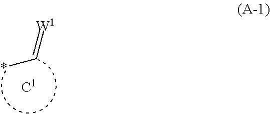

In Formula (A-1), W¹ represents an oxygen atom, a sulfur atom, ═NR^{W1}, or ═CR^{W2}R^{W3}.

R^{W1} represents a hydrogen atom or a substituent. Examples of the substituent include the groups exemplified as the substituent W.

R^{W2} and R^{W3} each independently represent a cyano group, —SO₂R^{W4}, —C(═O)OR^{W5}, or —C(═O)R^{W6}.

R^{W4} to R^{W6} each independently represent an aliphatic hydrocarbon group which may have a substituent, an aromatic ring group which may have a substituent, or an aliphatic heterocyclic group which may have a substituent.

Examples of the substituent which may be included in R^{W4} to R^{W6} include the groups exemplified as the substituent W.

The above-described aliphatic hydrocarbon group may be linear, branched, or cyclic, and preferably has 1 to 3 carbon atoms.

The above-described aromatic ring group may be an aromatic hydrocarbon ring group or an aromatic heterocyclic group, and a phenyl group is preferable.

The number of ring members in the above-described aliphatic heterocyclic group is preferably 5 to 20, more preferably 5 to 12, and still more preferably 6 to 8.

Examples of a heteroatom which is included in the above-described aliphatic heterocyclic group include a sulfur atom, an oxygen atom, a nitrogen atom, a selenium atom, a tellurium atom, a phosphorus atom, a silicon atom, and a boron atom; and a sulfur atom, an oxygen atom, or a nitrogen atom is preferable.

Examples of an aliphatic heterocyclic ring constituting the above-described aliphatic heterocyclic group include a pyrrolidine ring, an oxolane ring, a thiolane ring, a piperidine ring, a tetrahydrofuran ring, a tetrahydropyran ring, a thiacine ring, a piperazine ring, a morpholine ring, a quinocyclidine ring, an azetidine ring, an oxetane ring, an aziridine ring, a dioxane ring, a pentamethylene sulfide ring, and a γ-butyrolactone ring.

From the viewpoint that the effect of the present invention is more excellent, W¹ is preferably an oxygen atom, a sulfur atom, or ═CR^{W2}R^{W3}, more preferably an oxygen atom or a sulfur atom, and still more preferably an oxygen atom.

In Formula (A-1), C¹ represents a ring which contains at least two carbon atoms and may have a substituent.

Two carbon atoms contained in C¹ are the two carbon atoms specified in Formula (A-1).

The number of carbon atoms in the above-described ring is preferably 3 to 30, more preferably 3 to 20, and still more preferably 3 to 10. The number of carbon atoms in the above-described ring is the number including the two carbon atoms specified in the formula.

The above-described ring may be an aromatic ring or a non-aromatic ring.

The above-described ring may be any of a monocyclic ring or a polycyclic ring, and is preferably a 5-membered ring, a 6-membered ring, or a fused ring including at least one of a 5-membered ring or a 6-membered ring. The number of carbon atoms in the above-described fused ring including at least one of a 5-membered ring or a 6-membered ring is preferably 6 to 20, more preferably 6 to 15, and still more preferably 8 to 10.

The above-described ring may have a heteroatom. Examples of the heteroatom include a nitrogen atom, a sulfur atom, an oxygen atom, a selenium atom, a tellurium atom, a phosphorus atom, a silicon atom, and a boron atom; and a sulfur atom, a nitrogen atom, or an oxygen atom is preferable.

The number of heteroatoms in the above-described ring is preferably 0 to 10 and more preferably 0 to 5.

Among the carbon atoms constituting the ring represented by C¹, a carbon atom other than the carbon atom at a bonding position to which * is attached in Formula (A-1) and the carbon atom bonded to W¹ may be replaced with a carbonyl carbon (>C═O) or a thiocarbonyl carbon (>C═S).

Examples of the substituent which may be included in the ring include the groups exemplified as the substituent W; and a halogen atom, an alkyl group, an aromatic ring group, or a silyl group is preferable, and a halogen atom or an alkyl group is more preferable.

The above-described alkyl group may be linear, branched, or cyclic, and is preferably linear.

The number of carbon atoms in the above-described alkyl group is preferably 1 to 10 and more preferably 1 to 3.

As the above-described ring represented by C¹, a ring used as an acidic nucleus (for example, an acidic nucleus of a merocyanine coloring agent) is preferable; and examples thereof include the following nuclei.
(a) 1,3-dicarbonyl nucleus: for example, 1,3-indandione nucleus, 1,3-cyclohexanedione, 5,5-dimethyl-1,3-cyclohexanedione, 1,3-dioxane-4,6-dione, and the like
(b) pyrazolinone nucleus: for example, 1-phenyl-2-pyrazolin-5-one, 3-methyl-1-phenyl-2-pyrazolin-5-one, 1-(2-benzothiazolyl)-3-methyl-2-pyrazolin-5-one, and the like
(c) isoxazolinone nucleus: for example, 3-phenyl-2-isoxazolin-5-one, 3-methyl-2-isoxazolin-5-one, and the like
(d) oxindole nucleus: for example, 1-alkyl-2,3-dihydro-2-oxindole and the like
(e) 2,4,6-trioxohexahydropyrimidine nucleus: for example, barbituric acid, 2-thibarbituric acid and derivatives thereof, and the like; examples of the above-described derivatives include a 1-alkyl form such as 1-methyl and 1-ethyl, a 1,3-dialkyl form such as 1,3-dimethyl, 1,3-diethyl, and 1,3-dibutyl, a 1,3-diaryl form such as 1,3-diphenyl, 1,3-di(p-chlorophenyl), 1,3-di(p-ethoxycarbonylphenyl), a 1-alkyl-1-aryl form such as 1-ethyl-3-phenyl, and a 1,3-diheteroaryl form such as 1,3-di(2-pyridyl).
(f) 2-thio-2,4-thiazolidinedione nucleus: for example, rhodanine and derivatives thereof, and the like; examples of the above-described derivatives include a 3-alkylrhodanine such as 3-methylrhodanine, 3-ethylrhodanine, and 3-allylrhodanine, a 3-arylrhodanine such as 3-phenylrhodanine, and a 3-heteroaryl rhodanine such as 3-(2-pyridyl) rhodanine.
(g)₂-thio-2,4-oxazolidinedione (2-thio-2,4-(3H,5H)-oxazoledione) nucleus: for example, 3-ethyl-2-thio-2,4-oxazolidinedione and the like
(h) thianaphthenone nucleus: for example, 3 (2H)-thianaphthenone-1,1-dioxide and the like
(i) 2-thio-2,5-thiazolidinedione nucleus: for example, 3-ethyl-2-thio-2,5-thiazolidinedione and the like
(j) 2,4-thiazolidinedione nucleus: for example, 2,4-thiazolidinedione, 3-ethyl-2,4-thiazolidinedione, 3-phenyl-2,4-thiazolidinedione, and the like
(k) thiazolin-4-one nucleus: for example, 4-thiazolinone, 2-ethyl-4-thiazolinone, and the like
(l) 2,4-imidazolidinedione (hydantoin) nucleus: for example, 2,4-imidazolidinedione, 3-ethyl-2,4-imidazolidinedione, and the like
(i) 2-thio-2,4-imidazolidinedione (2-thiohydantoine) nucleus: for example, 2-thio-2,4-imidazolidinedione, 3-ethyl-2-thio-2,4-imidazolidinedione, and the like
(n) imidazolin-5-one nucleus: for example, 2-propylmercapto-2-imidazolin-5-one and the like
(o) 3,5-pyrazolidinedione nucleus: for example, 1,2-diphenyl-3,5-pyrazolidinedione, 1,2-dimethyl-3,5-pyrazolidinedione, and the like
(p) benzothiophen-3 (2H)-one nucleus: for example, benzothiophen-3 (2H)-one, oxobenzothiophen-3 (2H)-one, dioxobenzothiophen-3 (2H)-one, and the like
(q) indanone nucleus: for example, 1-indanone, 3-phenyl-1-indanone, 3-methyl-1-indanone, 3,3-diphenyl-1-indanone, 3,3-dimethyl-1-indanone, and the like
(r) benzofuran-3-(2H)-one nucleus: for example, benzofuran-3-(2H)-one and the like(s) 2,2-dihydrophenalene-1,3-dione nucleus and the like

From the viewpoint that the effect of the present invention is more excellent, A¹ and A² are each independently preferably a group represented by Formula (A-2).

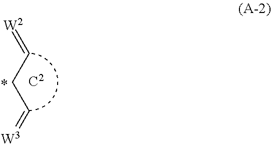

In Formula (A-2), W² and W³ each independently represent an oxygen atom, a sulfur atom, ═NR^{W1}, or ═CR^{W2}R^{W3}.

Definition and suitable aspects of R^{W1}, R^{W2}, and R^{W3} are as described above.

From the viewpoint that the effect of the present invention is more excellent, W² and W³ are preferably an oxygen atom, a sulfur atom, or ═C(CN)₂, more preferably an oxygen atom or a sulfur atom, and still more preferably an oxygen atom. Among these, it is preferable that at least one of W² or W³ is an oxygen atom, and it is more preferable that W² and W³ are oxygen atoms.

In Formula (A-2), C² represents a ring which contains at least three carbon atoms and may have a substituent.

Three carbon atoms contained in C² are the three carbon atoms specified in Formula (A-2).

The number of carbon atoms in the above-described ring is preferably 3 to 30, more preferably 3 to 20, and still more preferably 3 to 10. The number of carbon atoms in the above-described ring is the number including the three carbon atoms specified in the formula.

The above-described ring may be an aromatic ring or a non-aromatic ring.

The above-described ring may be any of a monocyclic ring or a polycyclic ring, and is preferably a 5-membered ring, a 6-membered ring, or a fused ring including at least one of a 5-membered ring or a 6-membered ring. The number of carbon atoms in the above-described fused ring including at least one of a 5-membered ring or a 6-membered ring is preferably 6 to 20, more preferably 6 to 15, and still more preferably 8 to 10.

The above-described ring may have a heteroatom. Examples of the heteroatom include a nitrogen atom, a sulfur atom, an oxygen atom, a selenium atom, a tellurium atom, a phosphorus atom, a silicon atom, and a boron atom; and a sulfur atom, a nitrogen atom, or an oxygen atom is preferable.

The number of heteroatoms in the above-described ring is preferably 0 to 10 and more preferably 0 to 5.

Among the carbon atoms constituting the ring represented by C², a carbon atom other than the carbon atom at a bonding position to which * is attached in Formula (A-2) and the carbon atom bonded to W² or W³ may be replaced with a carbonyl carbon (>C═O) or a thiocarbonyl carbon (>C═S).

A suitable aspect of the substituent which may be included in the ring is the same as that of the substituent which may be included in the ring C¹ described above.

From the viewpoint that the effect of the present invention is more excellent, A¹ and A² are each independently more preferably a group represented by Formula (C-1) or a group represented by Formula (C-2), and still more preferably a group represented by Formula (C-2).

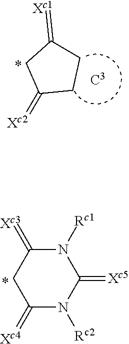

In Formula (C-1), X^{c1} and X^{c2} each independently represent an oxygen atom, a sulfur atom, ═NR^{X1}, or ═CR^{X2}R^{X3}.

R^{X1} represents a hydrogen atom or a substituent. Examples of the substituent represented by R^{X1} include the groups exemplified as the substituent W.

R^{X2} and R^{X3} each independently represent a cyano group, —SO₂R^{X4}, —C(═O)OR^{X5}, or —C(═O)R^{X6}.

R^{X4} to R^{X6} each independently represent an aliphatic hydrocarbon group which may have a substituent, an aromatic ring group which may have a substituent, or an aliphatic heterocyclic group which may have a substituent.

Definition and suitable aspects of each group represented by R^{X4} to R^{X6} are the same as those of each group represented by R^{W4} to R^{W6} described above.

It is preferable that at least one of R^{X2} or R^{X3} is a cyano group, and it is more preferable that R^{X2} and R^{X3} are cyano groups.

From the viewpoint that the effect of the present invention is more excellent, X^{c1} and X^{c2} are preferably an oxygen atom, a sulfur atom, or ═C(CN)₂, more preferably an oxygen atom or a sulfur atom, and still more preferably an oxygen atom. Among these, it is preferable that at least one of X^{c1} or X^{c2} is an oxygen atom, and it is more preferable that X^{c1} and X^{c2} are oxygen atoms.

In Formula (C-1), C³ represents an aromatic ring which may have a substituent.

The number of ring members in the above-described aromatic ring is preferably 4 to 30, more preferably 5 to 12, and still more preferably 5 to 8. The number of ring members in the above-described aromatic ring is the number including the two carbon atoms specified in the formula.

The above-described aromatic ring may be monocyclic or polycyclic.

In addition, the aromatic ring may be any of an aromatic hydrocarbon ring or an aromatic heterocyclic ring, and an aromatic hydrocarbon ring is preferable.

The aromatic ring represented by C³ is as described above; and is preferably a benzene ring, a naphthalene ring, an anthracene ring, a pyrene ring, a thiophene ring, a furan ring, a thiazole ring, an oxazole ring, a pyridine ring, a thienothiophene ring, a benzothiophene ring, a benzofuran ring, a pyrazine ring, a pyrimidine ring, or a pyridazine ring, more preferably a benzene ring, a naphthalene ring, or a thiophene ring, and particularly preferably a benzene ring.

Examples of the substituent which may be included in the above-described aromatic ring include the groups exemplified as the substituent W, and an alkyl group or a halogen atom is preferable.

The number of substituents which may be included in the above-described aromatic ring is not particularly limited, but is preferably 0 to 8 and more preferably 0 to 4.

In Formula (C-2), X^{c3} to X^{c5} each independently represent an oxygen atom, a sulfur atom, ═NR^{X1}, or ═CR^{X2}R^{X3}.

Definition and suitable aspects of R^{X1} to R^{X3} are as described above.

As X^{c3} to X^{c5}, an oxygen atom or a sulfur atom is preferable, and an oxygen atom is more preferable. Among these, it is preferable that at least two of X^{c3}, X^{c4}, or X^{c5} are oxygen atoms, and it is more preferable that all of X^{c3} to X^{c5} are oxygen atoms.

R^{c1} and R^{c2} each independently represent a hydrogen atom or a substituent.

Examples of the substituent include the groups exemplified as the substituent W; and an alkyl group or an aryl group is preferable, and an alkyl group is more preferable.

The above-described alkyl group may be linear, branched, or cyclic, and is preferably linear. The number of carbon atoms in the above-described alkyl group is preferably 1 to 20, more preferably 1 to 6, still more preferably 1 to 3, and particularly preferably 1.

The above-described aryl group may be monocyclic or polycyclic, and a phenyl group is preferable. The above-described phenyl group may further have a substituent, and examples of the substituent include the groups exemplified as the substituent W.

In Formula (1), in a case where A¹ and A² are the groups represented by Formula (A-2), the specific compound is represented by Formula (1-A2); in a case where A¹ and A² are the groups represented by Formula (C-1), the specific compound is represented by Formula (1-C1); and in a case where A¹ and A² are the groups represented by Formula (C-2), the specific compound is represented by Formula (1-C2).

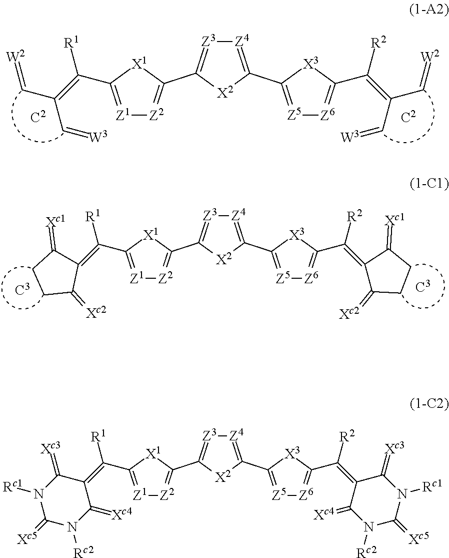

The specific compound is preferably the compound represented by Formula (1-A2), more preferably the compound represented by Formula (1-C1) or the compound represented by Formula (1-C2), and still more preferably the compound represented by Formula (1-C2).

In Formula (1-A2), Formula (1-C1), and Formula (1-C2), groups represented by the same reference numerals, which are present in a plural number, may be the same or different from each other, but are preferably the same.

Specific examples of the specific compound are shown below, but the present invention is not limited thereto.

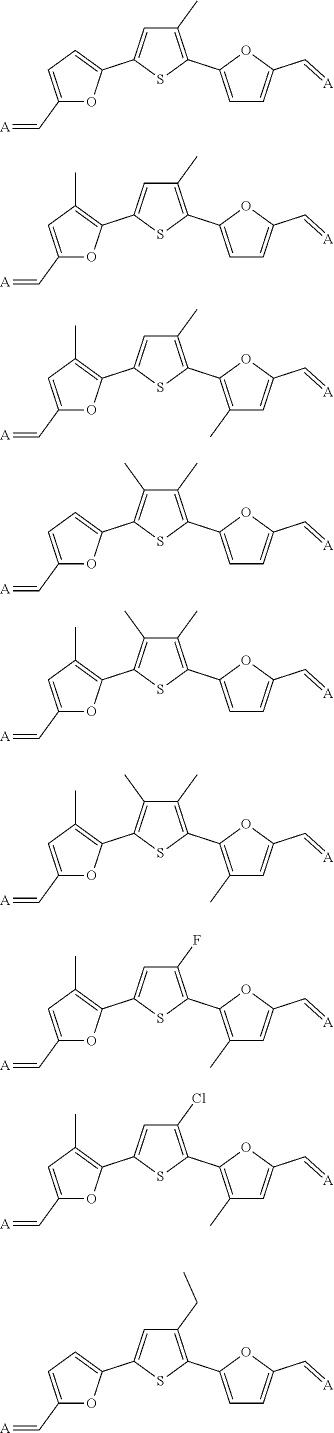

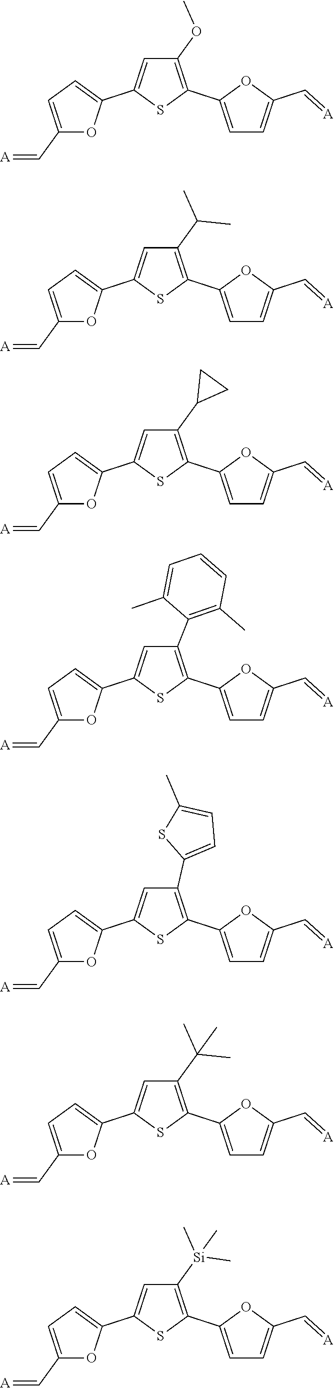

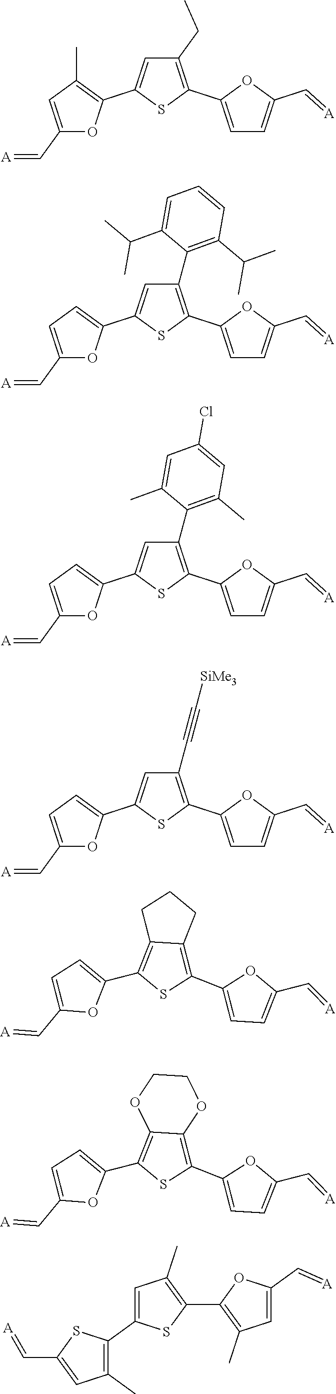

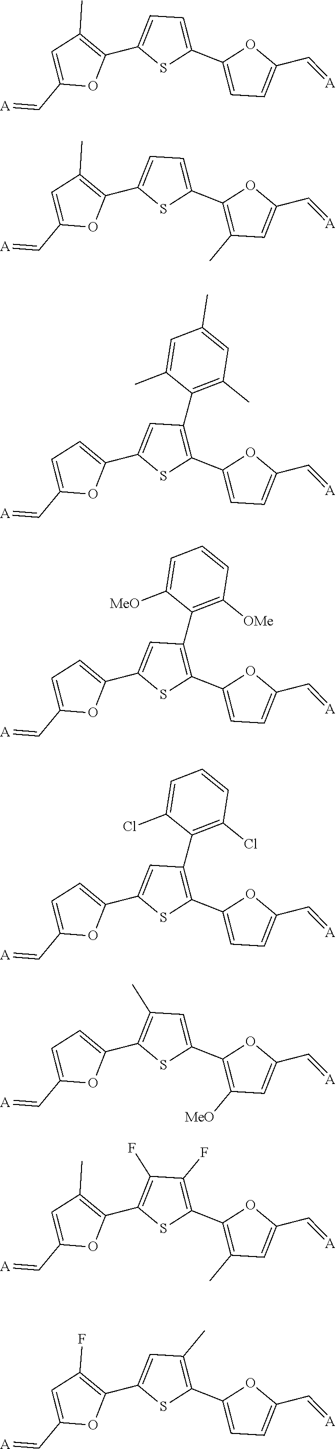

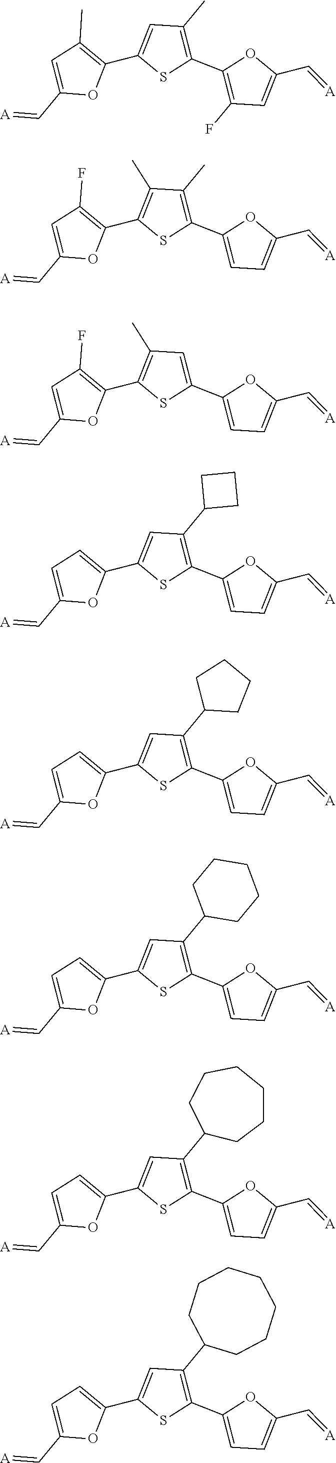

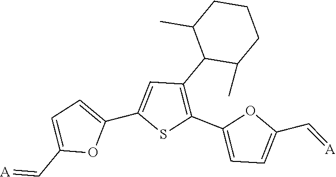

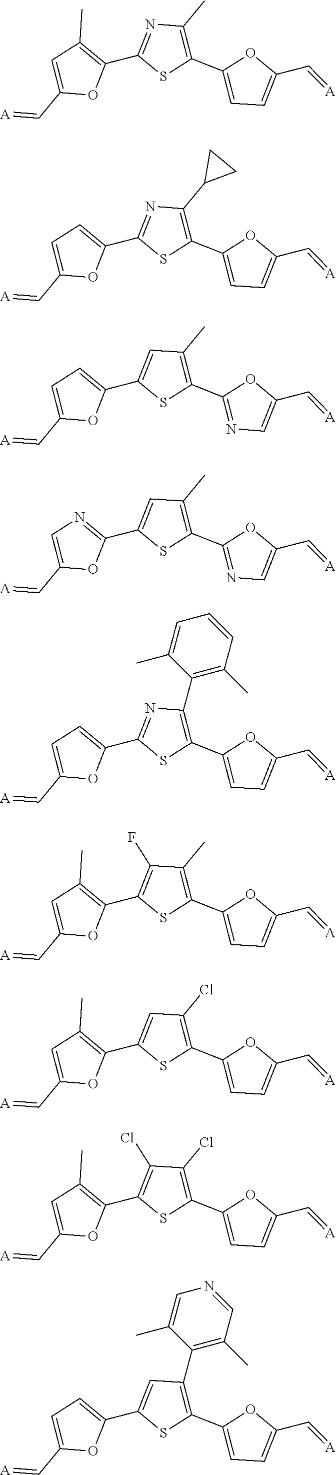

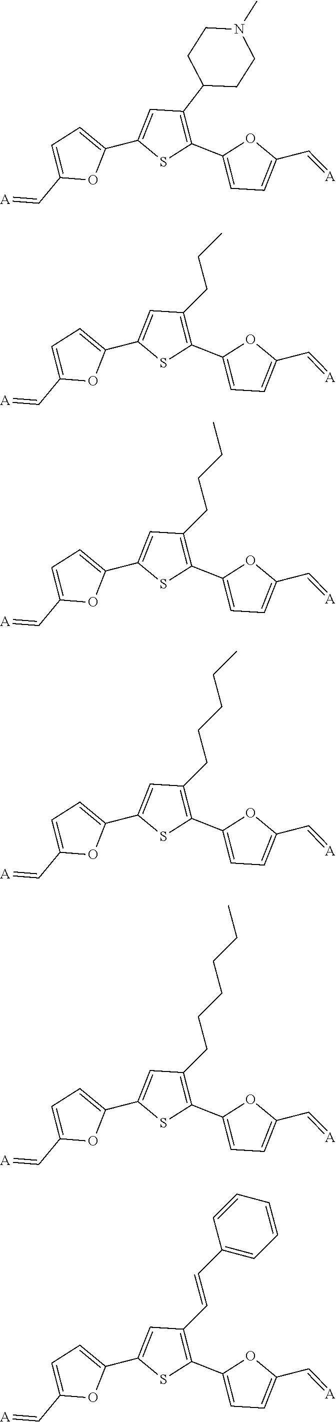

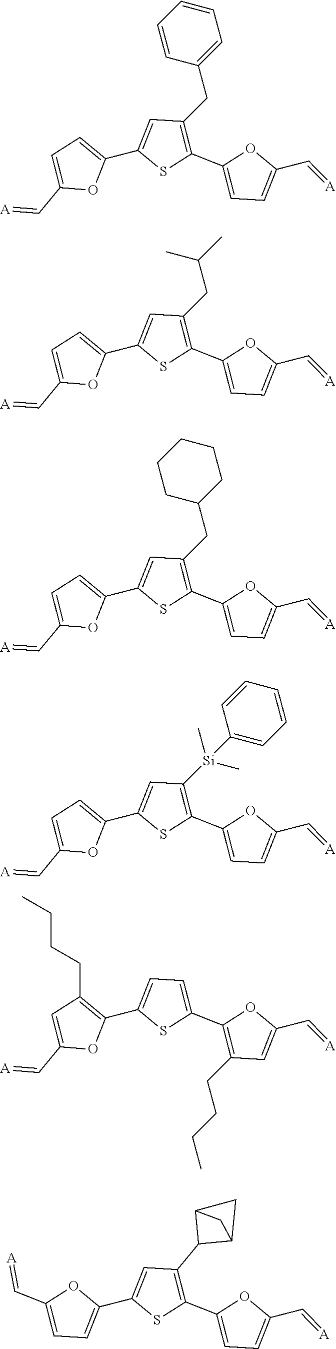

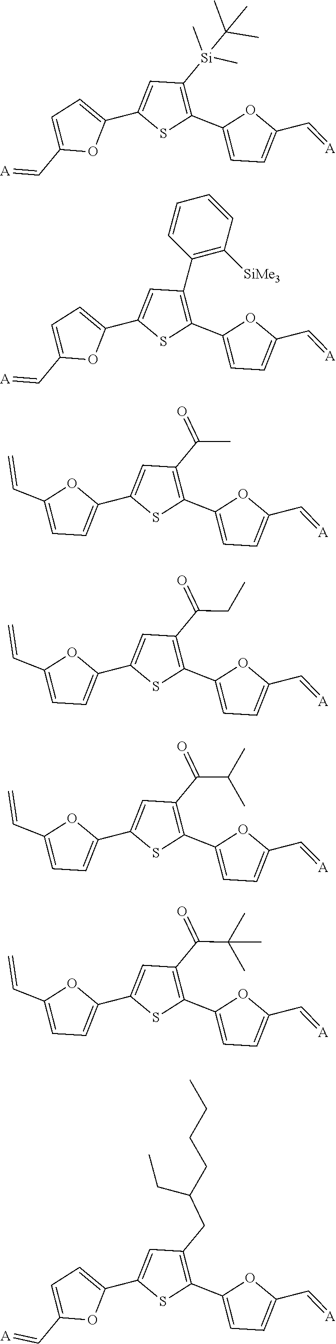

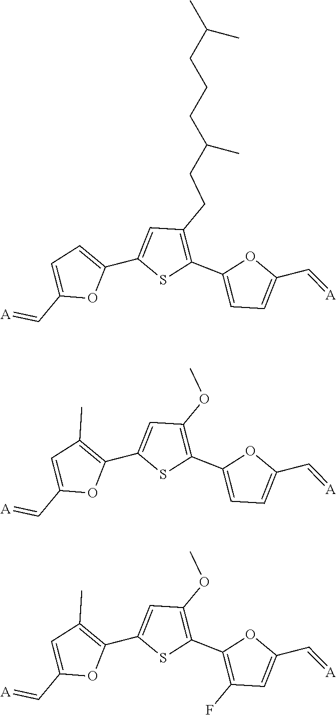

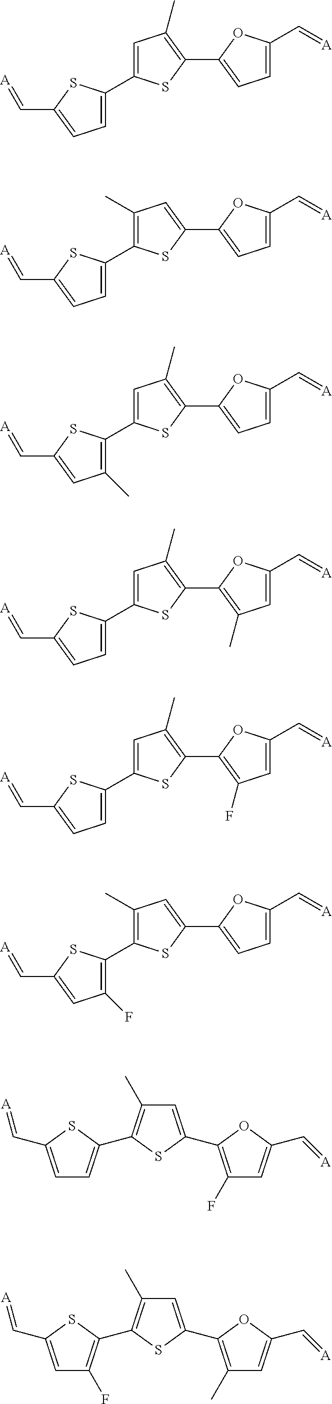

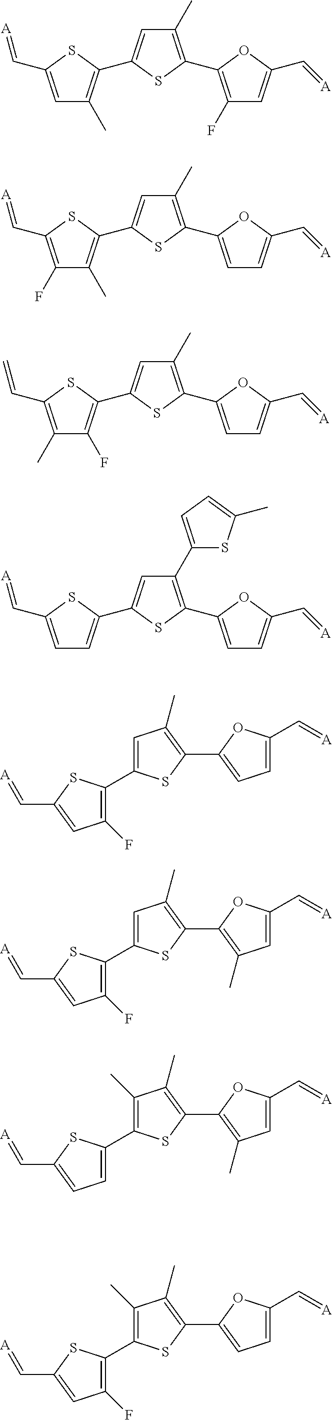

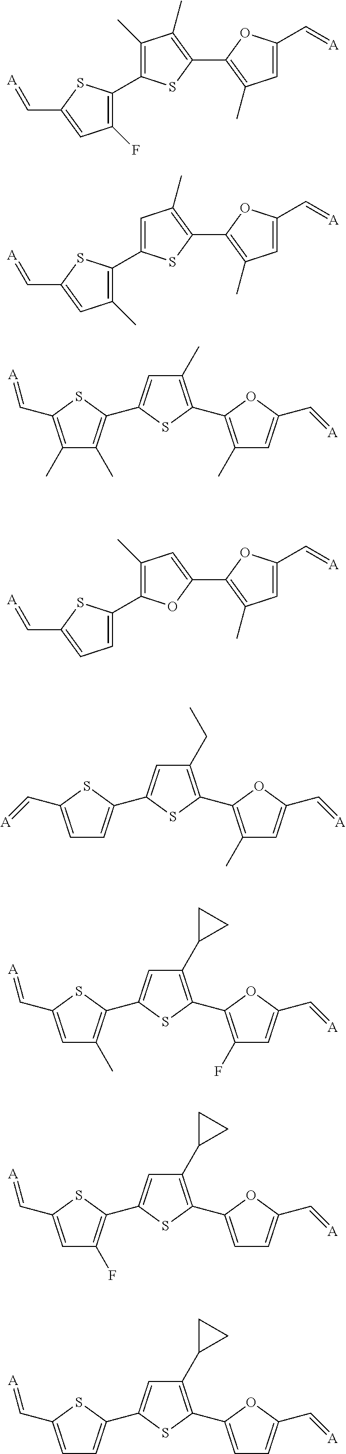

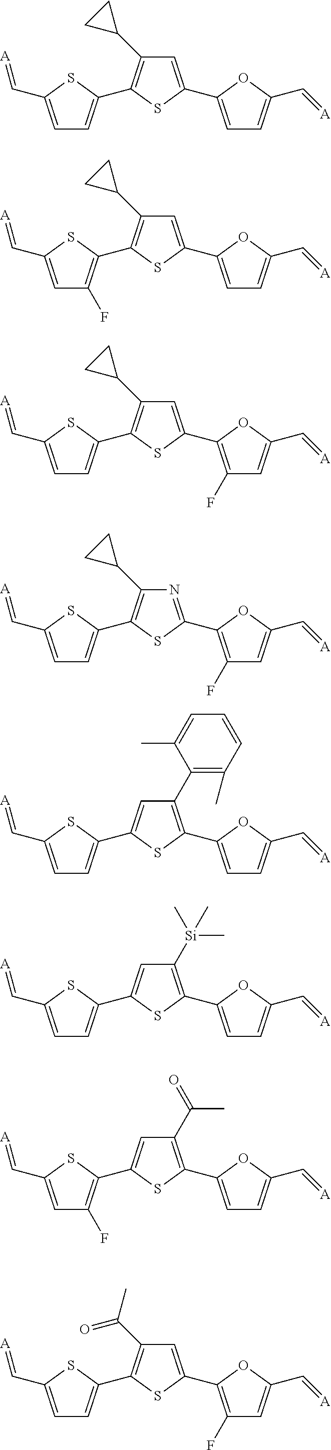

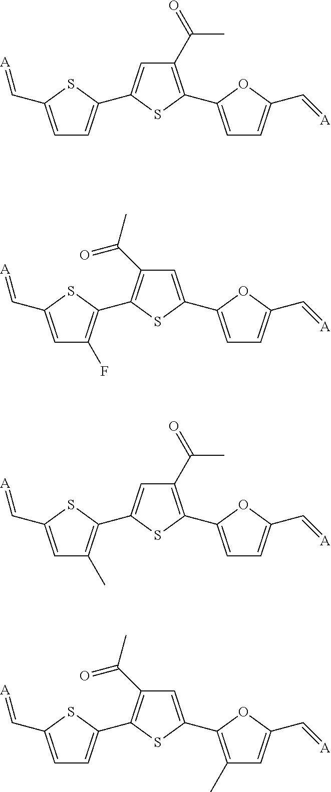

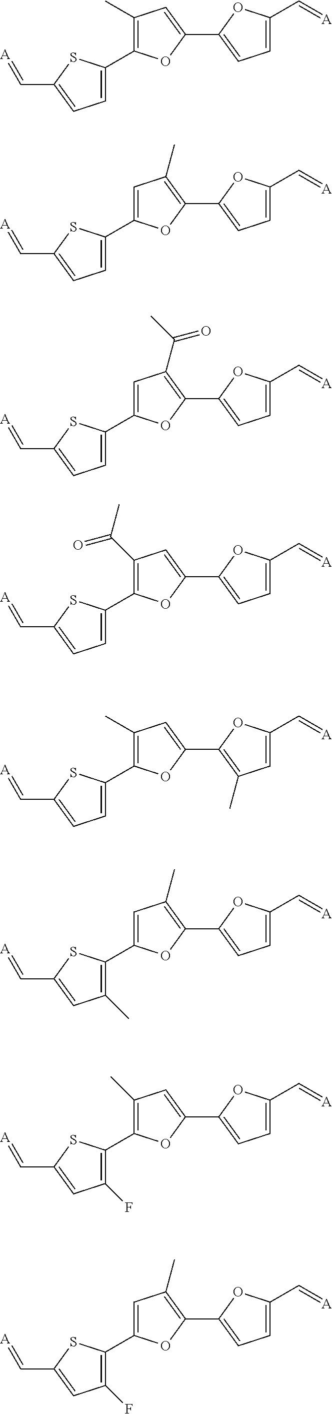

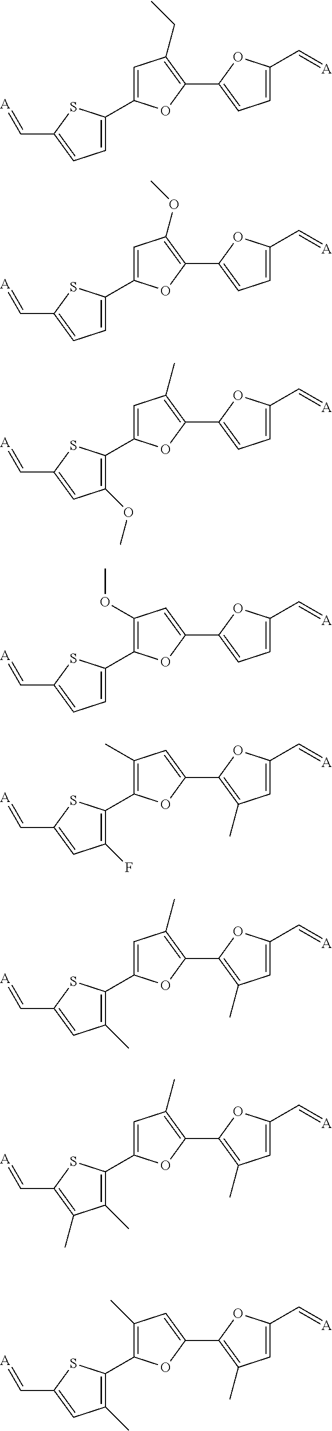

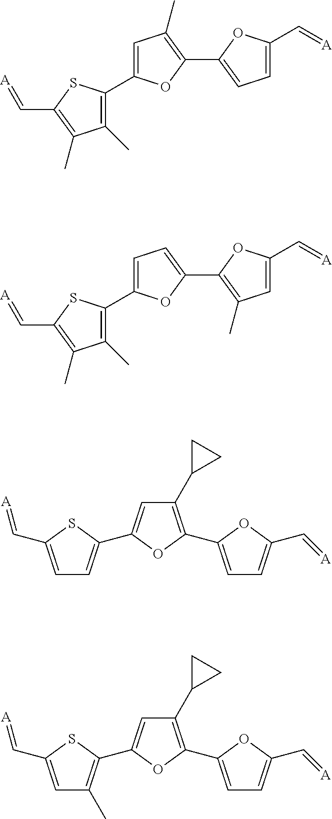

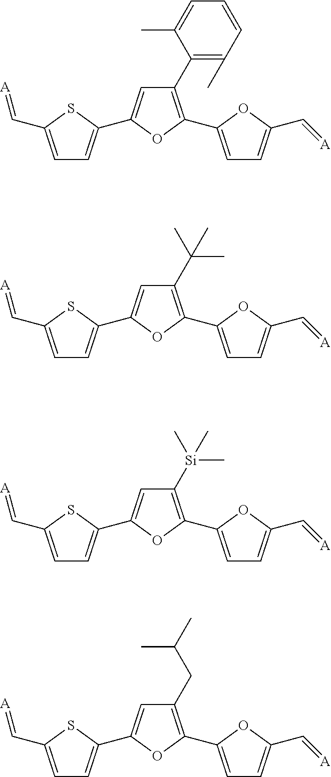

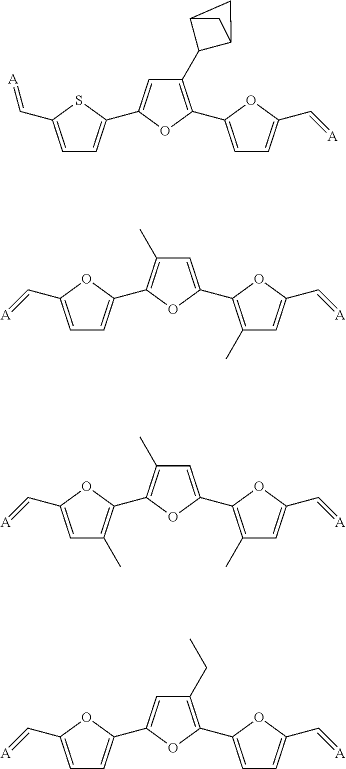

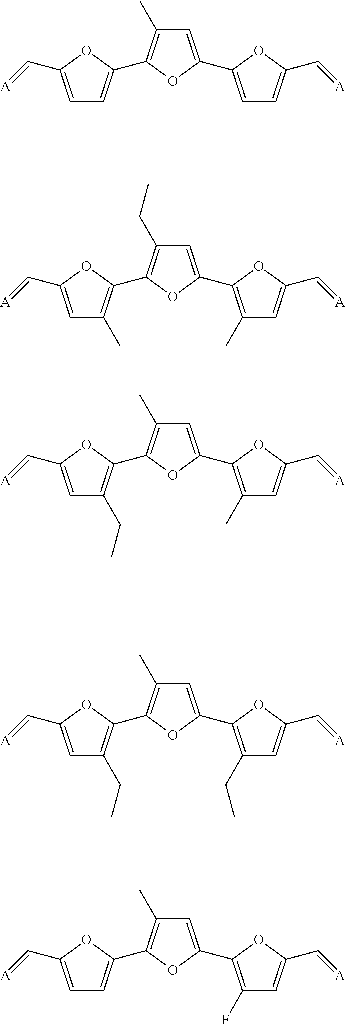

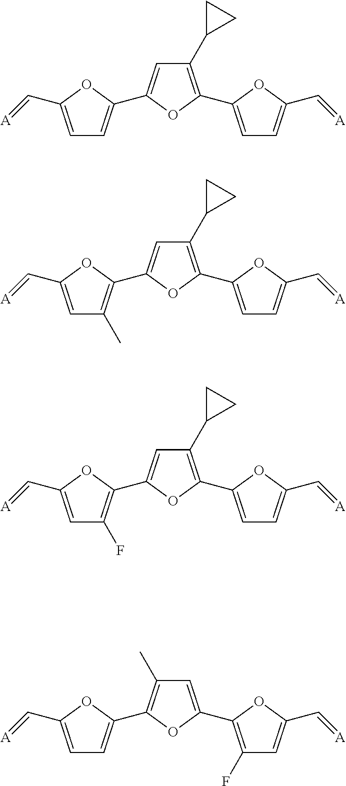

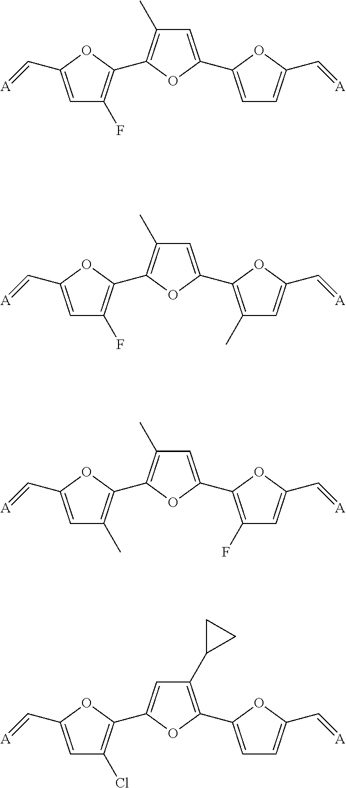

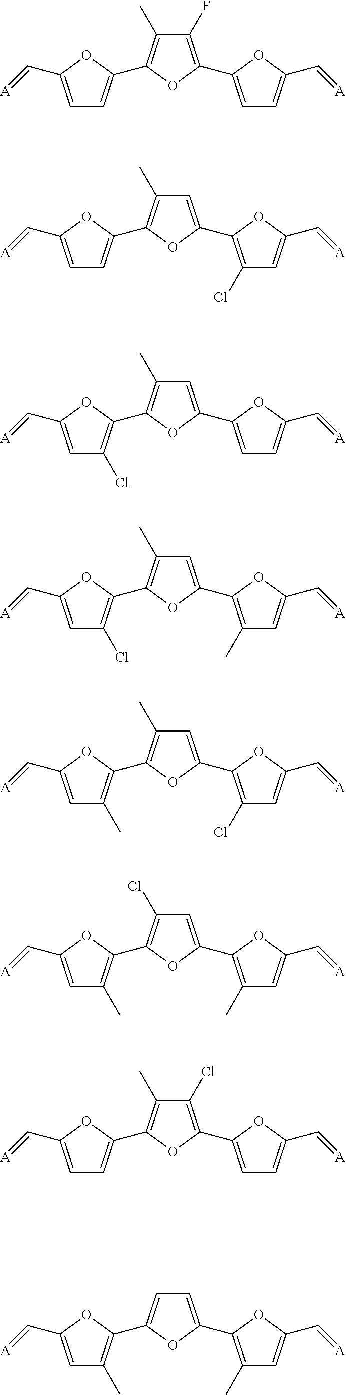

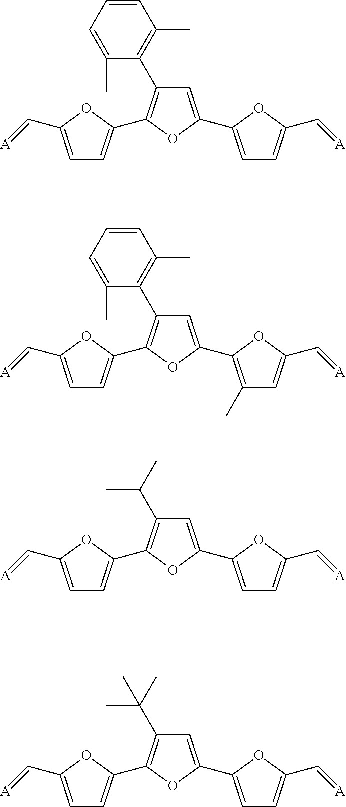

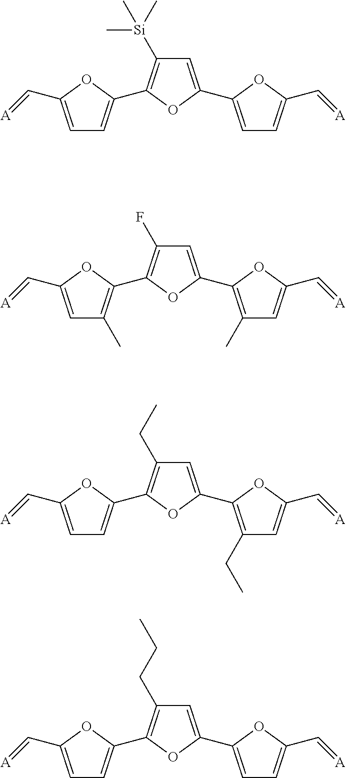

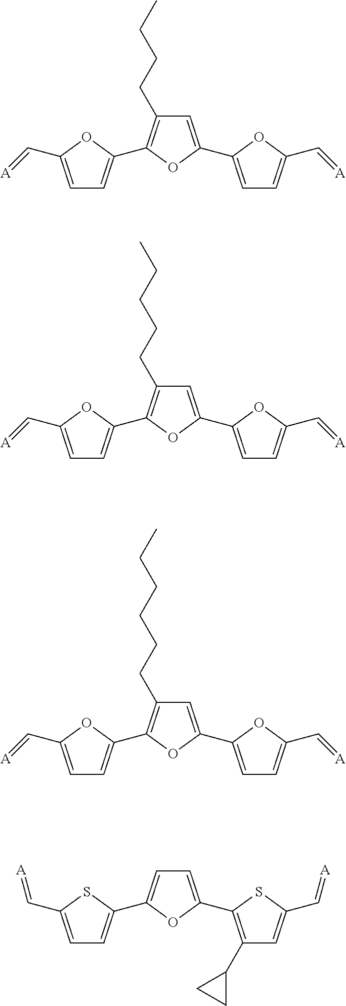

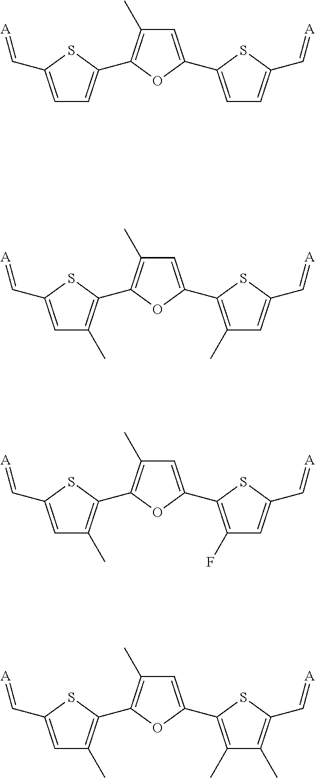

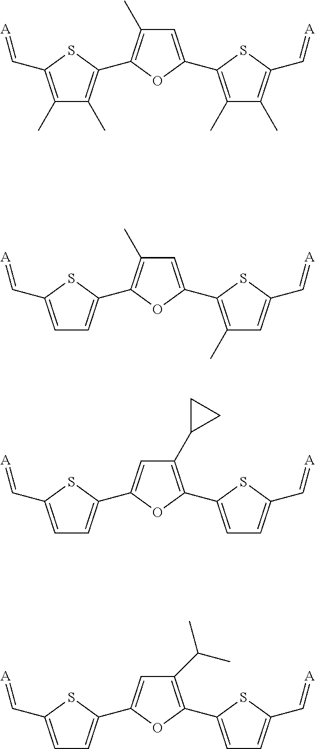

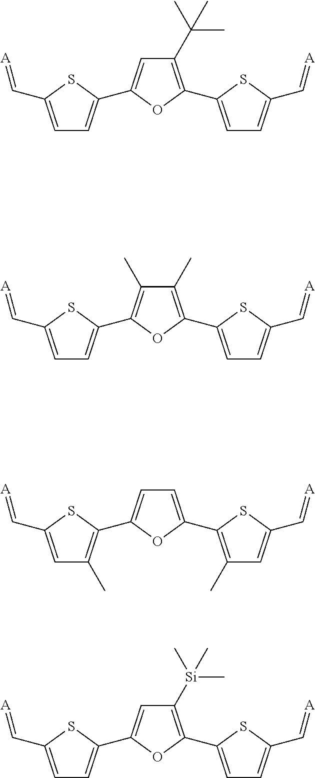

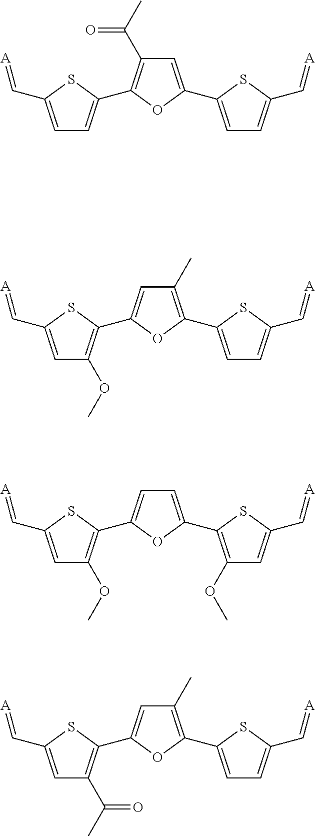

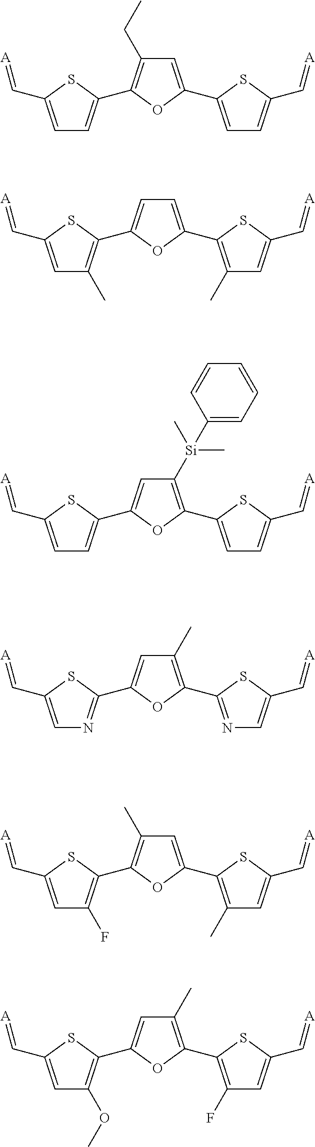

A in the specific compounds exemplified above is represented by any of the following groups.

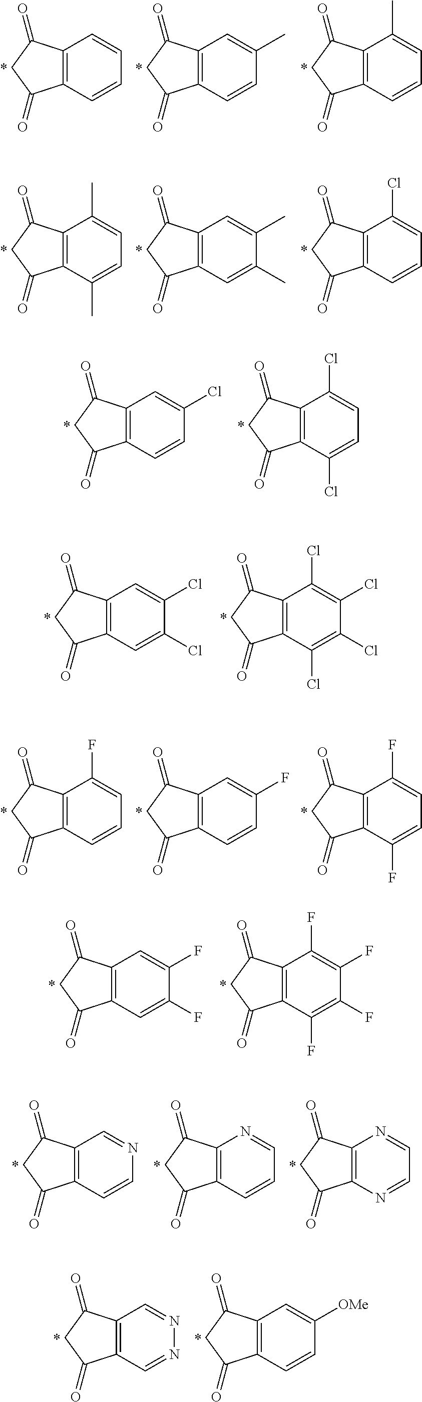

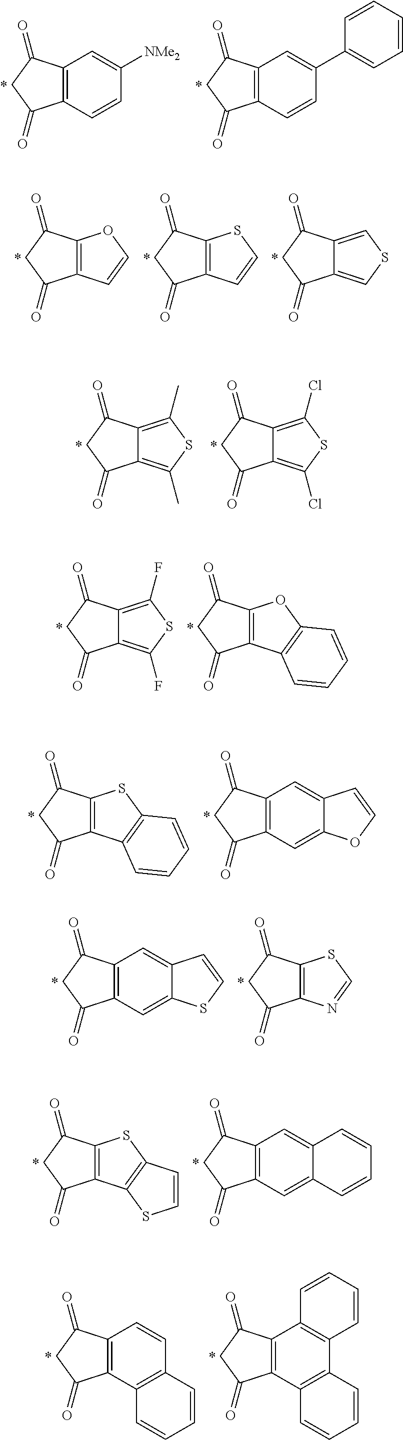

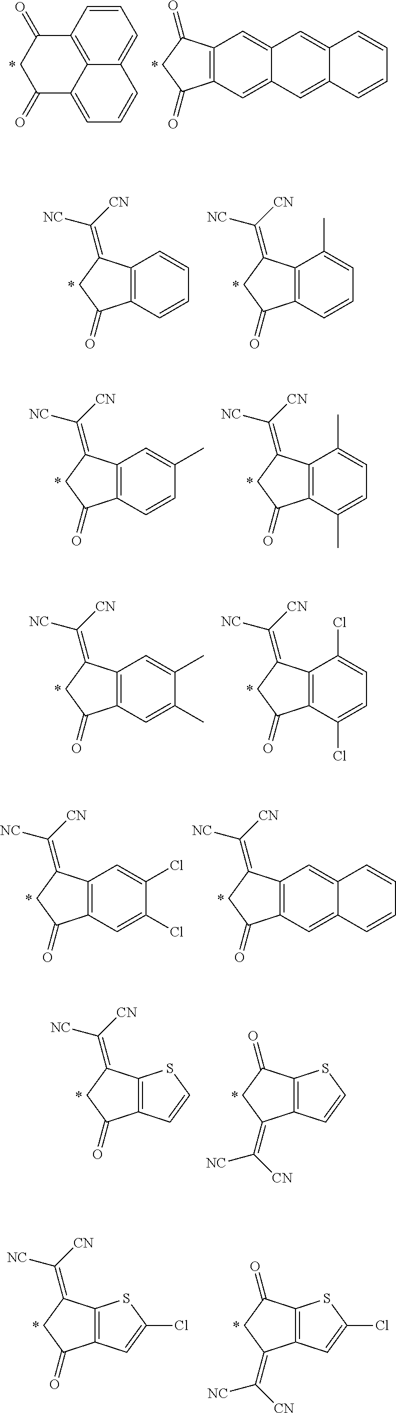

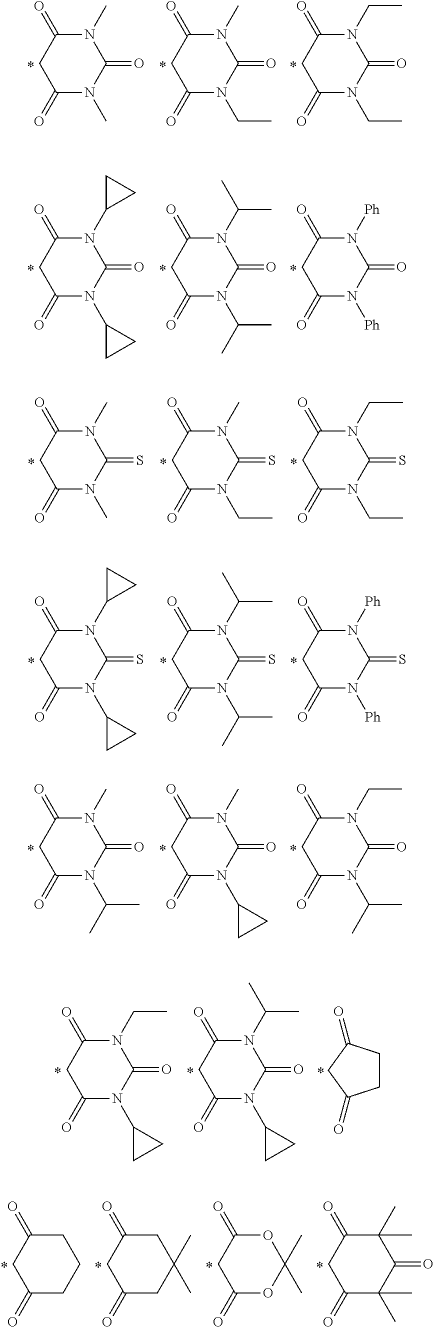

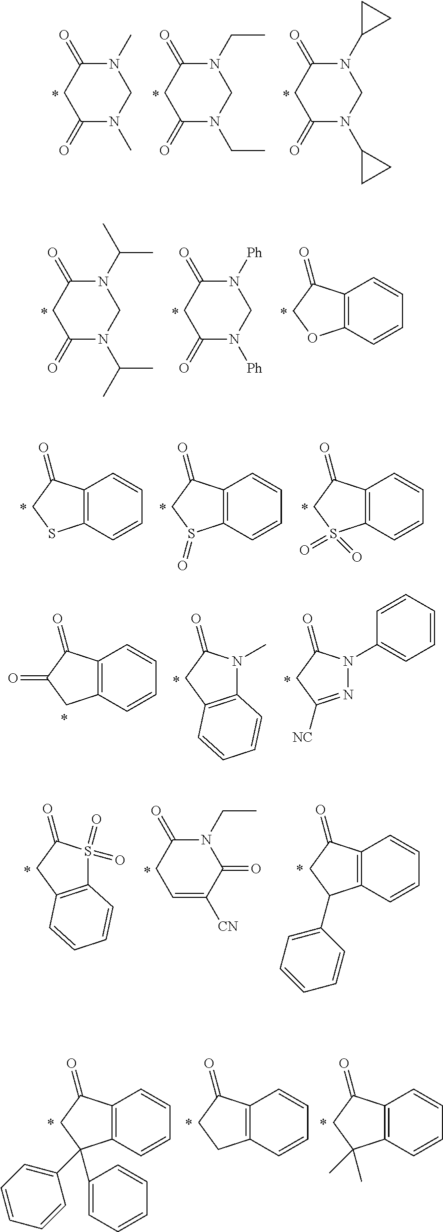

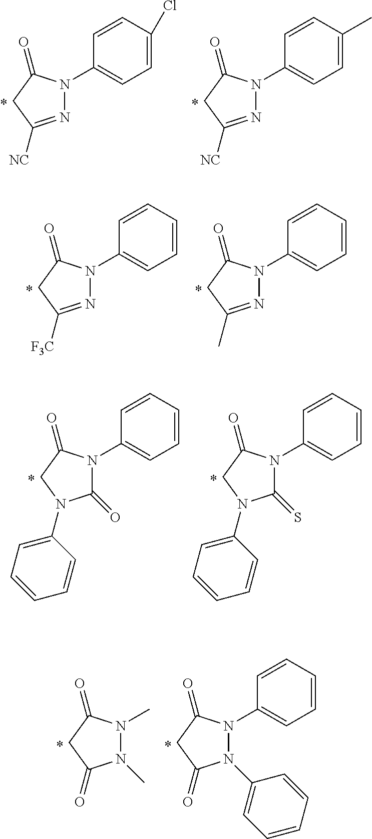

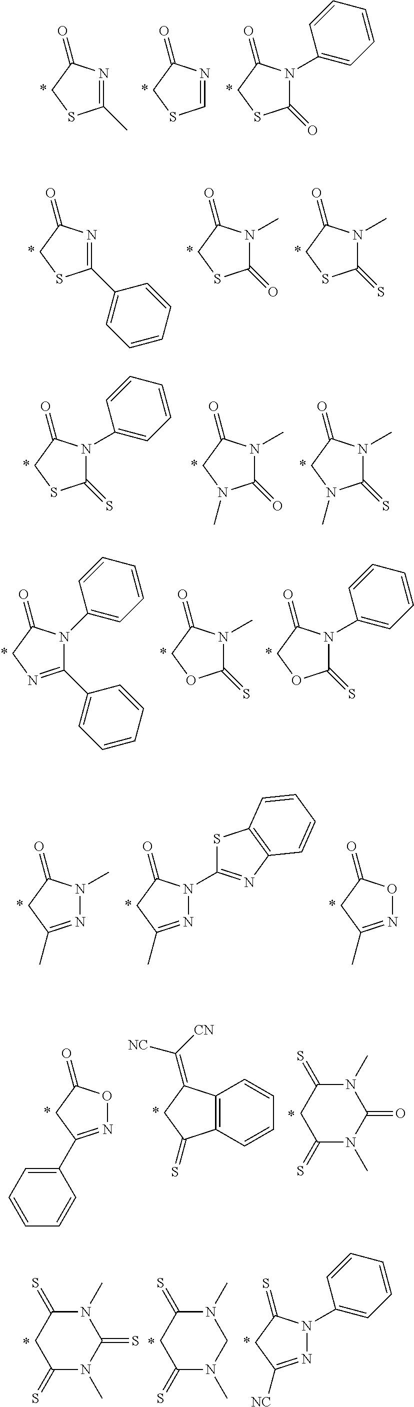

A molecular weight of the specific compound is preferably 400 to 1,400, more preferably 480 to 1,000, and still more preferably 520 to 800.

In a case where the molecular weight is in the above-described range, it is presumed that sublimation temperature of the specific compound is low, and thus the manufacturing suitability is excellent.

From the viewpoint of stability in a case of using the specific compound as a p-type organic semiconductor and matching of energy levels between the specific compound and an n-type organic semiconductor, an ionization potential of the specific compound in a single film is preferably −6.0 to −5.0 eV.

A maximal absorption wavelength of the specific compound is preferably in a wavelength range of 400 to 700 nm, and more preferably in a wavelength range of 450 to 650 nm.

The above-described maximal absorption wavelength is a value measured in a solution state (solvent: chloroform) by adjusting the absorption spectrum of the specific compound to a concentration such that the light absorbance is 0.5 to 1.0. However, in a case where the specific compound is not soluble in chloroform, a value measured by using the specific compound in which the specific compound is vapor-deposited and formed into a film state is defined as the maximal absorption wavelength of the specific compound.

The specific compound is particularly useful as a material of a photoelectric conversion film used for an imaging element, an optical sensor, or a photoelectric cell. The specific compound usually functions as a coloring agent in the photoelectric conversion film. In addition, the specific compound can also be used as a coloring material, a liquid crystal material, an organic semiconductor material, a charge transport material, a pharmaceutical material, and a fluorescent diagnostic material.

The specific compound may be purified as necessary.

Examples of a purification method of the specific compound include sublimation purification, purification using silica gel column chromatography, purification using gel permeation chromatography, re-slurry washing, re-purification by re-precipitation, purification using an adsorbent such as activated carbon, and recrystallization purification.

A content of the specific compound in the photoelectric conversion film (=Film thickness of specific compound in terms of single layer/Film thickness of photoelectric conversion film×100) is not particularly limited, but is preferably 5% to 75% by volume, more preferably 10% to 50% by volume, and still more preferably 15% to 40% by volume.

The specific compound may be used alone or in combination of two or more types thereof. In a case where two or more types thereof are used, it is preferable that the total amount thereof is within the above-described range.

<n-Type Organic Semiconductor>

The photoelectric conversion film preferably contains an n-type organic semiconductor, in addition to the specific compound.

The n-type organic semiconductor is a compound different from the above-described specific compound.

The n-type organic semiconductor is an acceptor-type organic semiconductor material (compound), and refers to an organic compound having a property of easily accepting an electron. That is, the n-type organic semiconductor refers to an organic compound having a larger electron affinity in a case where two organic compounds used in contact with each other. That is, any organic compound having an electron accepting property can be used as the acceptor-type organic semiconductor.

Examples of the n-type organic semiconductor include fullerenes selected from the group consisting of a fullerene and derivatives thereof; fused aromatic carbocyclic compounds (for example, a naphthalene derivative, an anthracene derivative, a phenanthrene derivative, a tetracene derivative, a pyrene derivative, a perylene derivative, a fluoranthene derivative, and the like); heterocyclic compounds with a 5- to 7-membered ring having at least one selected from the group consisting of a nitrogen atom, an oxygen atom, and a sulfur atom (for example, pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, thiazole, and the like); polyarylene compounds; fluorene compounds; cyclopentadiene compounds; silyl compounds; 1,4,5,8-naphthalenetetracarboxylic acid anhydride; 1,4,5,8-naphthalenetetracarboxylic acid anhydride imide derivatives and oxadiazole derivatives; anthraquinodimethane derivatives; diphenylquinone derivatives; bathocuproine, bathophenanthroline, and derivatives thereof; triazole compounds; distyrylarylene derivatives; metal complexes having a nitrogen-containing heterocyclic compound as a ligand; silole compounds; and compounds described in paragraphs [0056] and [0057] of JP2006-100767A.

The n-type organic semiconductor (compound) is preferably fullerenes selected from the group consisting of a fullerene and derivatives thereof.

Examples of the fullerene include a fullerene C₆₀, a fullerene C₇₀, a fullerene C₇₆, a fullerene C₇₈, a fullerene C₈₀, a fullerene C₈₂, a fullerene C₈₄, a fullerene C₉₀, a fullerene C₉₆, a fullerene C₂₄₀, a fullerene C₅₄₀, and a mixed fullerene.

Examples of the derivatives of fullerene include compounds in which a substituent is added to the above-described fullerenes. The above-described substituent is preferably an alkyl group, an aryl group, or a heterocyclic group. As the derivatives of fullerene, compounds described in JP2007-123707A are preferable.

A molecular weight of the n-type organic semiconductor is preferably 200 to 1,200 and more preferably 200 to 900.

A maximal absorption wavelength of the n-type organic semiconductor is preferably in a wavelength of 400 nm or less or in a wavelength range of 500 to 600 nm.

It is preferable that the photoelectric conversion film has a bulk hetero structure formed in a state in which the specific compound and the n-type organic semiconductor are mixed with each other. The bulk hetero structure refers to a layer in which the specific compound and the n-type organic semiconductor are mixed and dispersed in the photoelectric conversion film. The photoelectric conversion film having the bulk hetero structure can be formed by a wet method or a dry method. The bulk hetero structure is as described in detail in paragraphs [0013] and [0014] of JP2005-303266A.

A difference in electron affinity between the specific compound and the n-type organic semiconductor is preferably 0.1 eV or more.

The n-type organic semiconductor may be used alone or in combination of two or more types thereof.

In a case where the photoelectric conversion film contains the n-type organic semiconductor, a content of the n-type organic semiconductor in the photoelectric conversion film (Film thickness of n-type organic semiconductor in terms of single layer/Film thickness of photoelectric conversion film×100) is preferably 15% to 75% by volume, more preferably 20% to 60% by volume, and still more preferably 20% to 50% by volume.

In a case where the n-type organic semiconductor includes fullerenes, a content of the fullerenes to the total content of the n-type organic semiconductor (Film thickness of fullerenes in terms of single layer/Total film thickness of n-type organic semiconductors in terms of single layer×100) is preferably 50% to 100% by volume, and more preferably 80% to 100% by volume. The fullerenes may be used alone or in combination of two or more types thereof.

From the viewpoint of response speed of the photoelectric conversion element, the content of the specific compound to the total content of the specific compound and the n-type organic semiconductor (Film thickness of specific compound in terms of single layer/(Film thickness of specific compound in terms of single layer+Film thickness of n-type organic semiconductor in terms of single layer)×100) is preferably 20% to 80% by volume, and more preferably 40% to 80% by volume.

In a case where the photoelectric conversion film contains the n-type organic semiconductor and a p-type organic semiconductor, the content of the specific compound (Film thickness of specific compound in terms of single layer/(Film thickness of specific compound in terms of single layer+Film thickness of n-type organic semiconductor in terms of single layer+Film thickness of p-type organic semiconductor in terms of single layer)×100) is preferably 10% to 75% by volume, and more preferably 15% to 50% by volume.

It is preferable that the photoelectric conversion film substantially contains the specific compound, the n-type organic semiconductor, and the p-type organic semiconductor contained as desired. The term “substantially” indicates that the total content of the specific compound, the n-type organic semiconductor, and the p-type organic semiconductor is 90% to 100% by volume, preferably 95% to 100% by volume, and more preferably 99% to 100% by volume with respect to the total volume of the photoelectric conversion film.

<p-Type Organic Semiconductor>

The photoelectric conversion film preferably contains a p-type organic semiconductor in addition to the above-described specific compound.

The p-type organic semiconductor is a compound different from the above-described specific compound.

The p-type organic semiconductor is a donor-type organic semiconductor material (compound), and refers to an organic compound having a property of easily donating an electron. That is, the p-type organic semiconductor refers to an organic compound having a smaller ionization potential in a case where two organic compounds are used in contact with each other.

The p-type organic semiconductor may be used alone or in combination of two or more types thereof.

Examples of the p-type organic semiconductor include triarylamine compounds (for example, N,N′-bis(3-methylphenyl)-(1,1′-biphenyl)-4,4′-diamine (TPD), 4,4′-bis[N-(naphthyl)-N-Phenyl-amino]biphenyl(α-NPD), compounds described in paragraphs [0128] to [0148] of JP2011-228614A, compounds described in paragraphs [0052] to [0063] of JP2011-176259A, compounds described in paragraphs [0119] to [0158] of JP2011-225544A, compounds described in paragraphs [0044] to [0051] of JP2015-153910A, compounds described in paragraphs [0086] to [0090] of JP2012-094660A, and the like), pyrazoline compounds, styrylamine compounds, hydrazone compounds, polysilane compounds, thiophene compounds (for example, a thienothiophene derivative, a dibenzothiophene derivative, a benzodithiophene derivative, a dithienothiophene derivative, a [1]benzothieno[3,2-b]thiophene (BTBT) derivative, a thieno[3,2-f:4,5-f′]bis[1]benzothiophene (TBBT) derivative, compounds described in paragraphs [0031] to [0036] of JP2018-014474A, compounds described in paragraphs [0043] to [0045] of WO2016/194630A, compounds described in paragraphs [0025] to [0037], and [0099] to [0109] of WO2017/159684A, compounds described in paragraphs [0029] to [0034] of JP2017-076766A, compounds described in paragraphs [0015] to [0025] of WO2018/207722A, compounds described in paragraphs [0045] to [0053] of JP2019-054228A, compounds described in paragraphs [0045] to [0055] of WO2019/058995A, compounds described in paragraphs [0063] to [0089] of WO2019/081416A, compounds described in paragraphs [0033] to [0036] of JP2019-80052A, compounds described in paragraphs [0044] to [0054] of WO2019/054125A, compounds described in paragraphs [0041] to [0046] of WO2019/093188A, compounds described in paragraphs [0034] to [0037] of JP2019-050398A, compounds described in paragraphs [0033] to [0036] of JP2018-206878A, compounds described n paragraph [0038] of JP2018-190755A, compounds described in paragraphs [0019] to [0021] of JP2018-026559A, compounds described in paragraphs [0031] to [0056] of JP2018-170487A, compounds described in paragraphs [0036] to [0041] of JP2018-078270A, compounds described in paragraphs [0055] to [0082] of JP2018-166200A, compounds described in paragraphs [0041] to [0050] of JP2018-113425A, compounds described in paragraphs [0044] to [0048] of JP2018-085430A, compounds described in paragraphs [0041] to [0045] of JP2018-056546A, compounds described in paragraphs [0042] to [0049] of JP2018-046267A, compounds described in paragraphs [0031] to [0036] of JP2018-014474A, compounds described in paragraphs [0036] to [0046] of WO2018/016465A, compounds described in paragraphs [0045] to [0048] of JP2020-010024A, and the like), a cyanine compound, an oxonol compound, a polyamine compound, an indole compound, a pyrrole compound, a pyrazole compound, a polyarylene compound, a fused aromatic carbocyclic compound (for example, a naphthalene derivative, an anthracene derivative, a phenanthrene derivative, a tetracene derivative, a pentacene derivative, a pyrene derivative, a perylene derivative, a fluoranthene derivative, and the like), a porphyrin compound, a phthalocyanine compound, a triazole compound, an oxadiazole compound, an imidazole compound, a polyarylalkane compound, a pyrazolone compound, an amino-substituted chalcone compound, an oxazole compound, a fluorenone compound, a silazane compound, and a metal complex having a nitrogen-containing heterocyclic compound as a ligand.

In addition, examples of the p-type organic semiconductor also include compounds described in JP2022-123944A, compounds described in JP2022-122839A, compounds described in JP2022-120323A, compounds described in JP2022-120273A, compounds described in JP2022-115832A, compounds described in JP2022-108268A, compounds described in JP2023-005703A, compounds described in JP2022-100258A, compounds described in JP2022-181226A, compounds described in JP2022-27575A, and compounds described in JP2021-163968A.

Examples of the p-type organic semiconductor also include compounds having an ionization potential smaller than that of the n-type organic semiconductor, and in a case where this condition is satisfied, the organic coloring agent exemplified as the n-type organic semiconductor can be used.

Compounds which can be used as the p-type organic semiconductor compound are exemplified below.

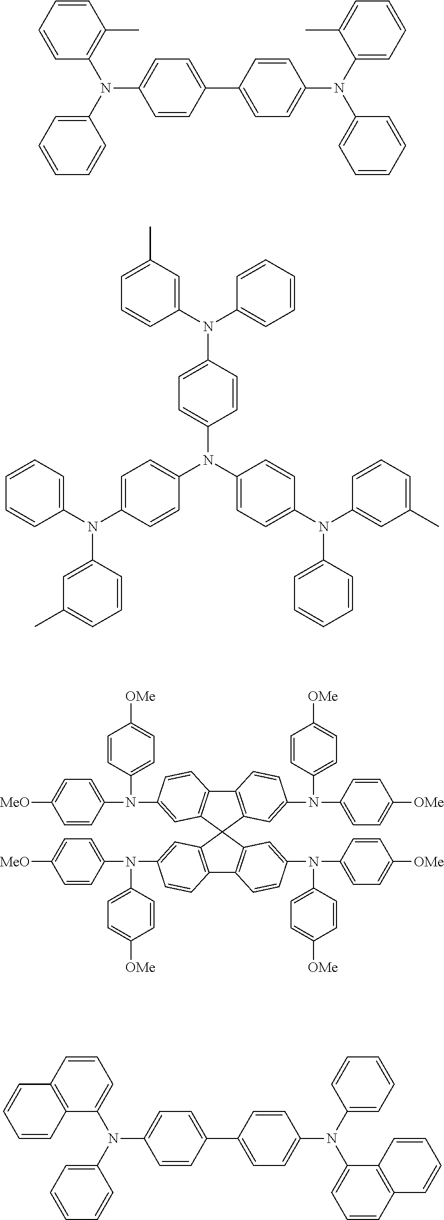 [SMILES: Cc1ccccc1N(c1ccccc1)c1ccc(-c2ccc(N(c3ccccc3)c3ccccc3C)cc2)cc1 ; Cc1cccc(N(c2ccccc2)c2ccc(N(c3ccc(N(c4ccccc4)c4cccc(C)c4)cc3)c3ccc(N(c4ccccc4)c4cccc(C)c4)cc3)cc2)c1 ; COc1ccc(N(c2ccc(OC)cc2)c2ccc3c(c2)C2(c4cc(N(c5ccc(OC)cc5)c5ccc(OC)cc5)ccc4-3)c3cc(N(c4ccc(OC)cc4)c4ccc(OC)cc4)ccc3-c3ccc(N(c4ccc(OC)cc4)c4ccc(OC)cc4)cc32)cc1 ; c1ccc(N(c2ccc(-c3ccc(N(c4ccccc4)c4cccc5ccccc45)cc3)cc2)c2cccc3ccccc23)cc1]

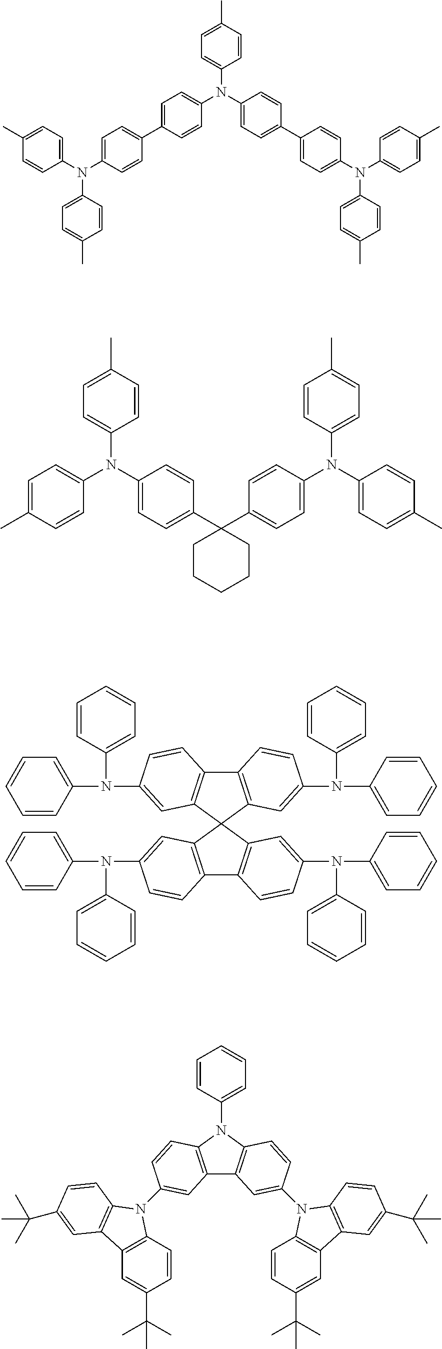

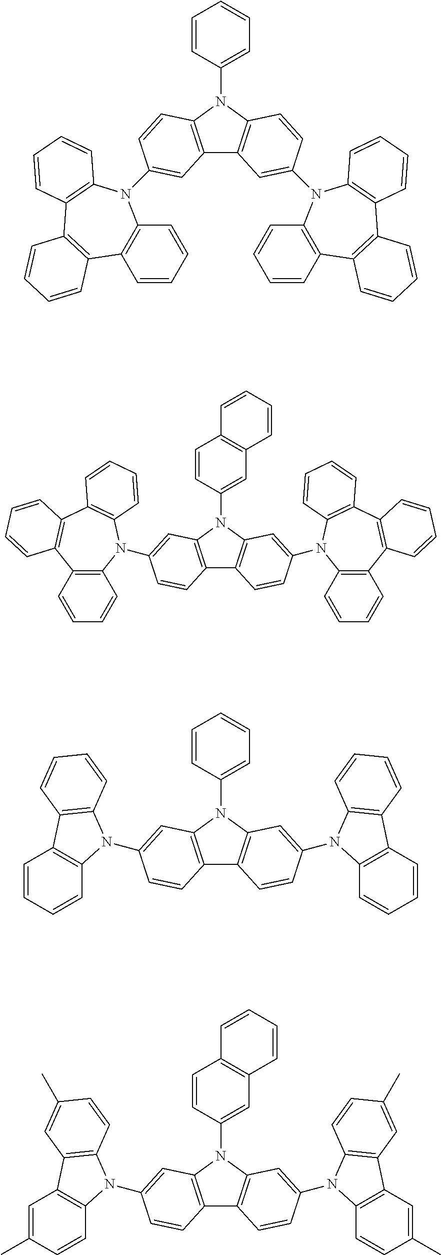

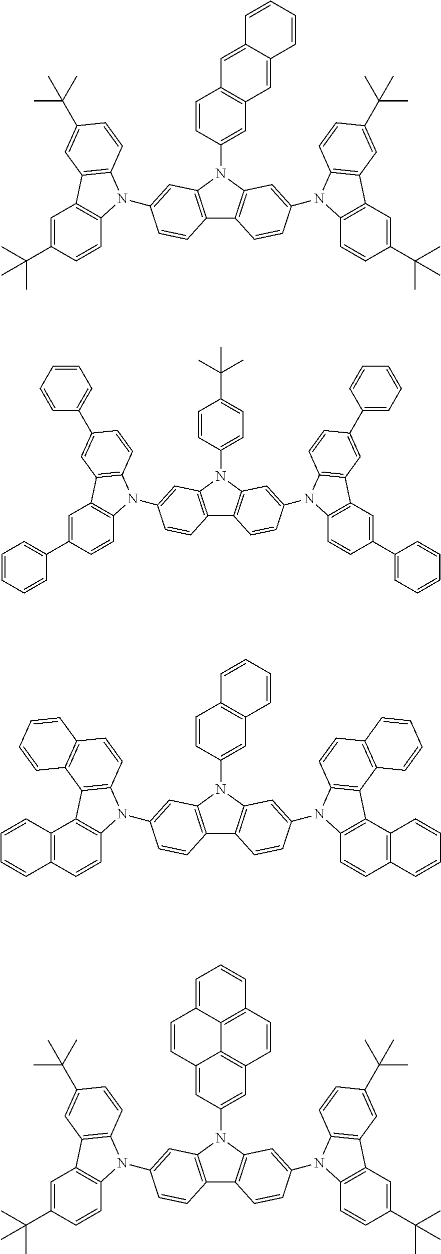

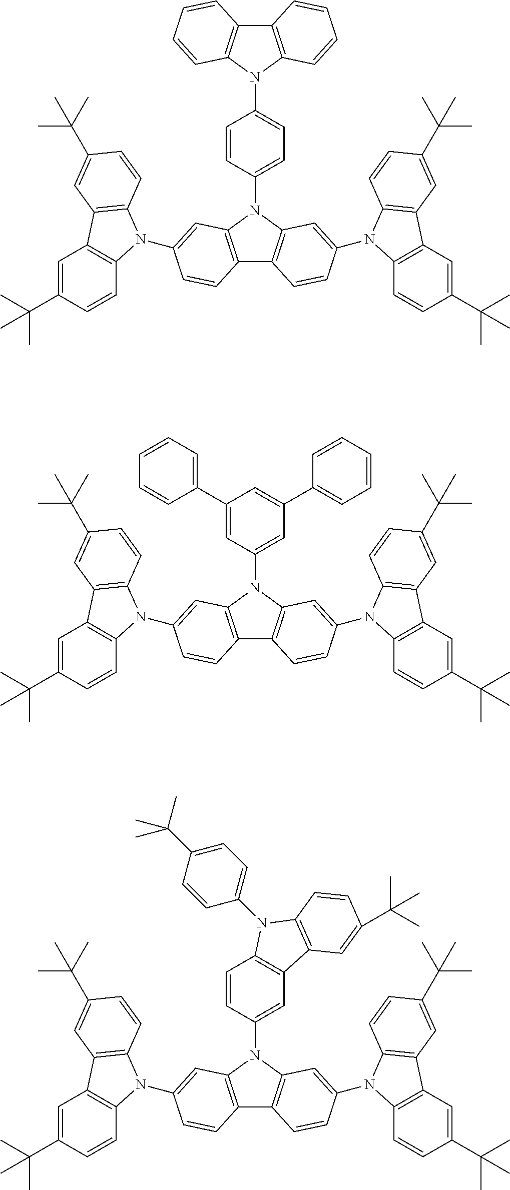

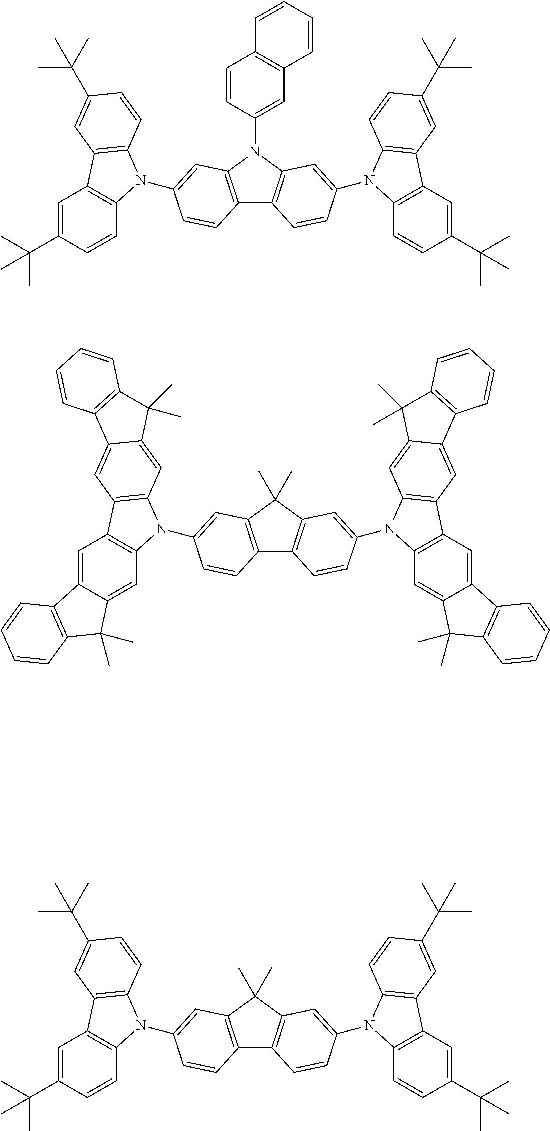

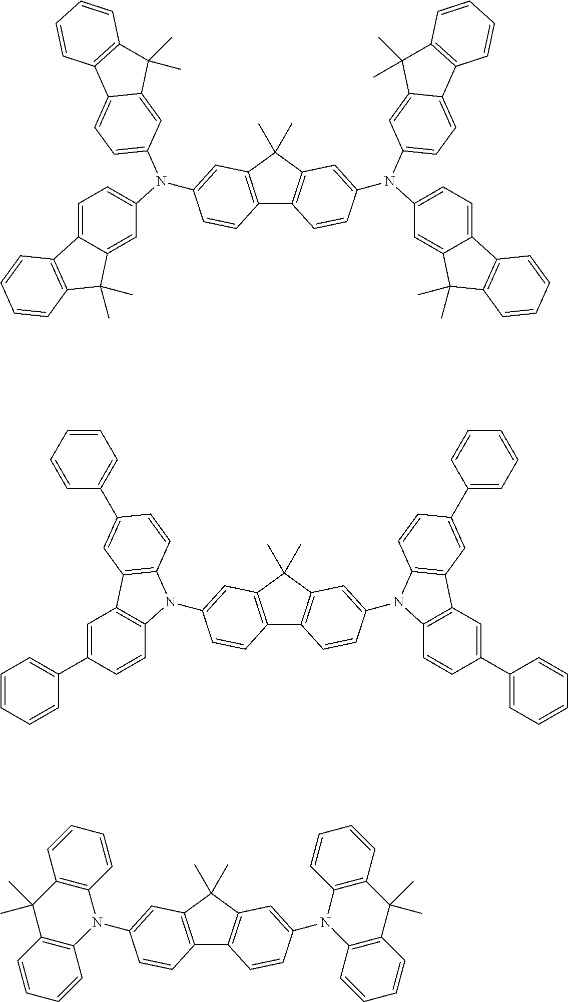

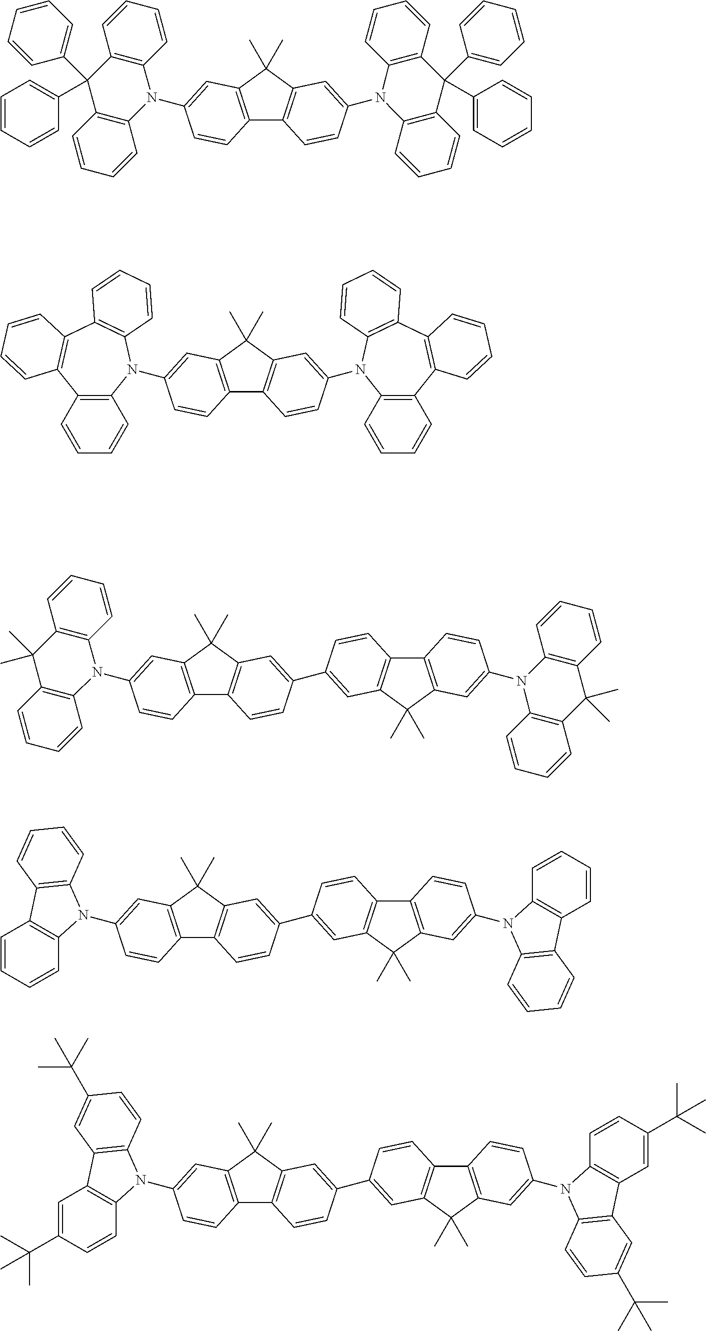

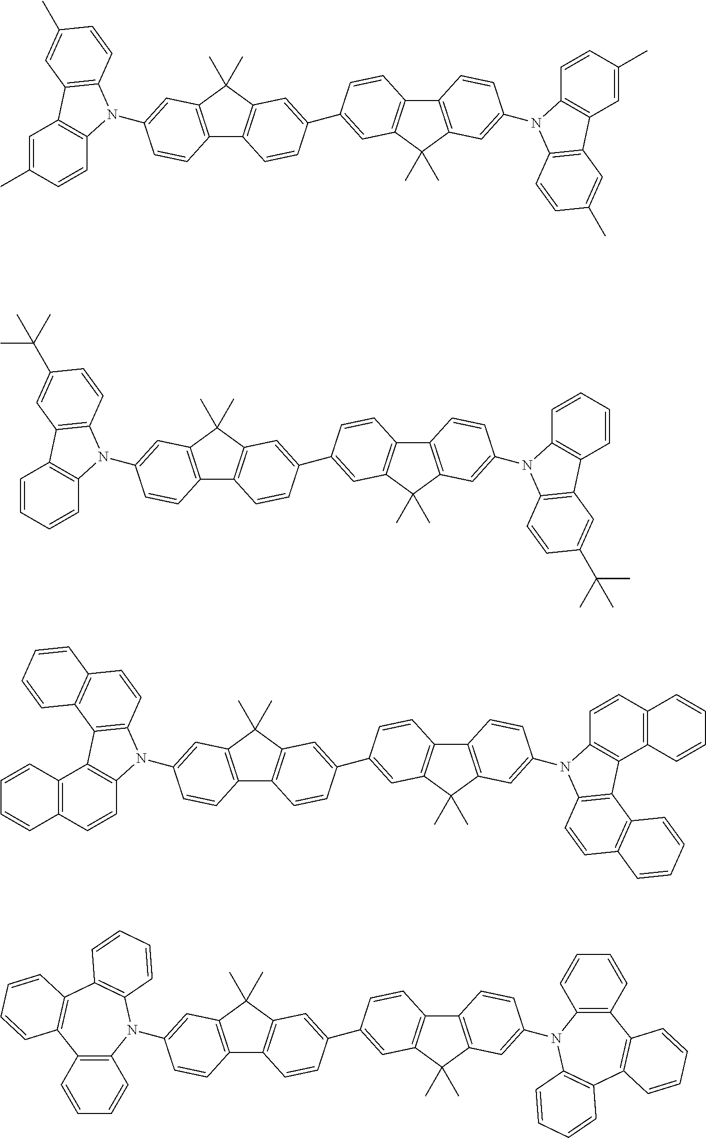

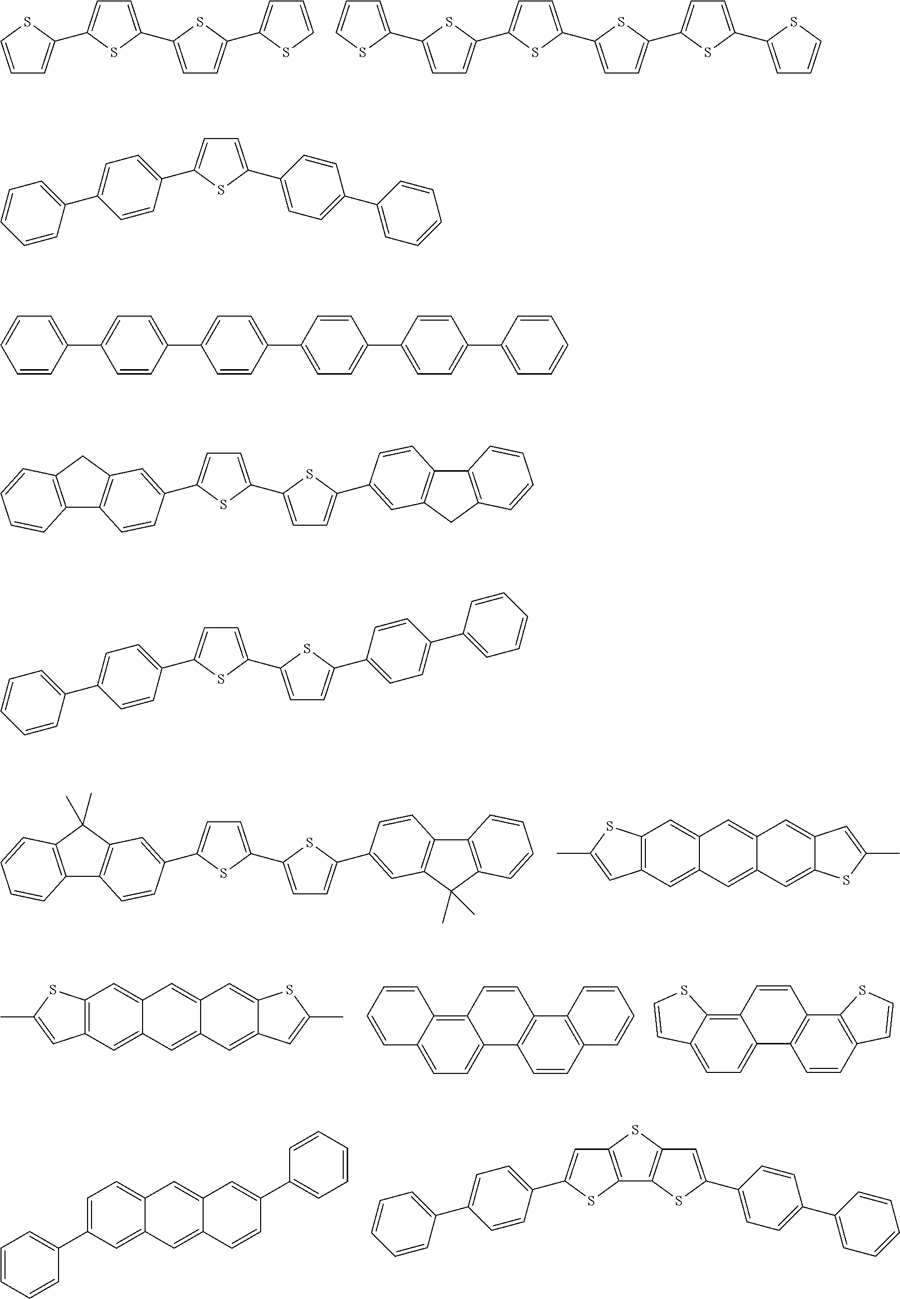

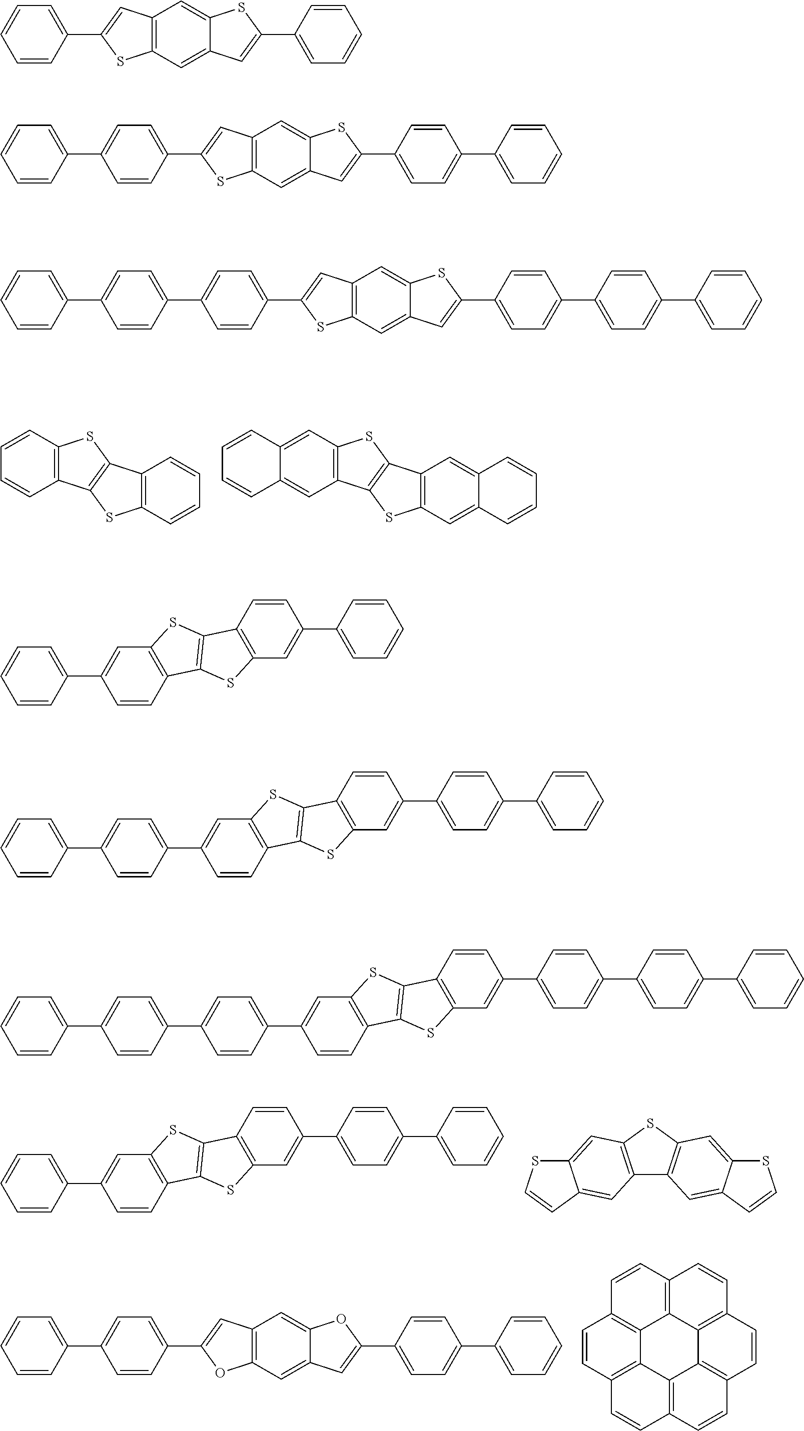

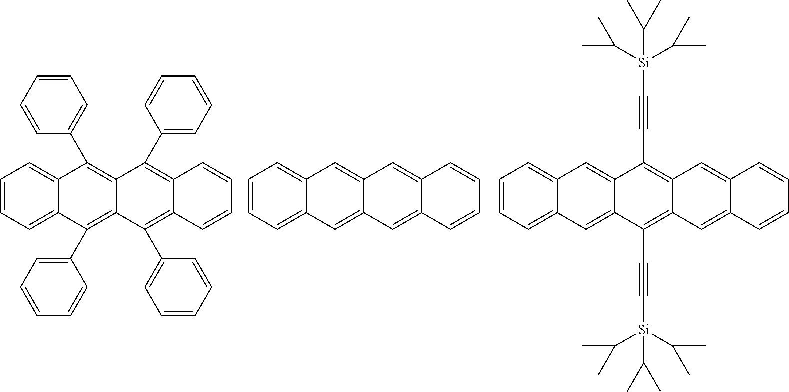

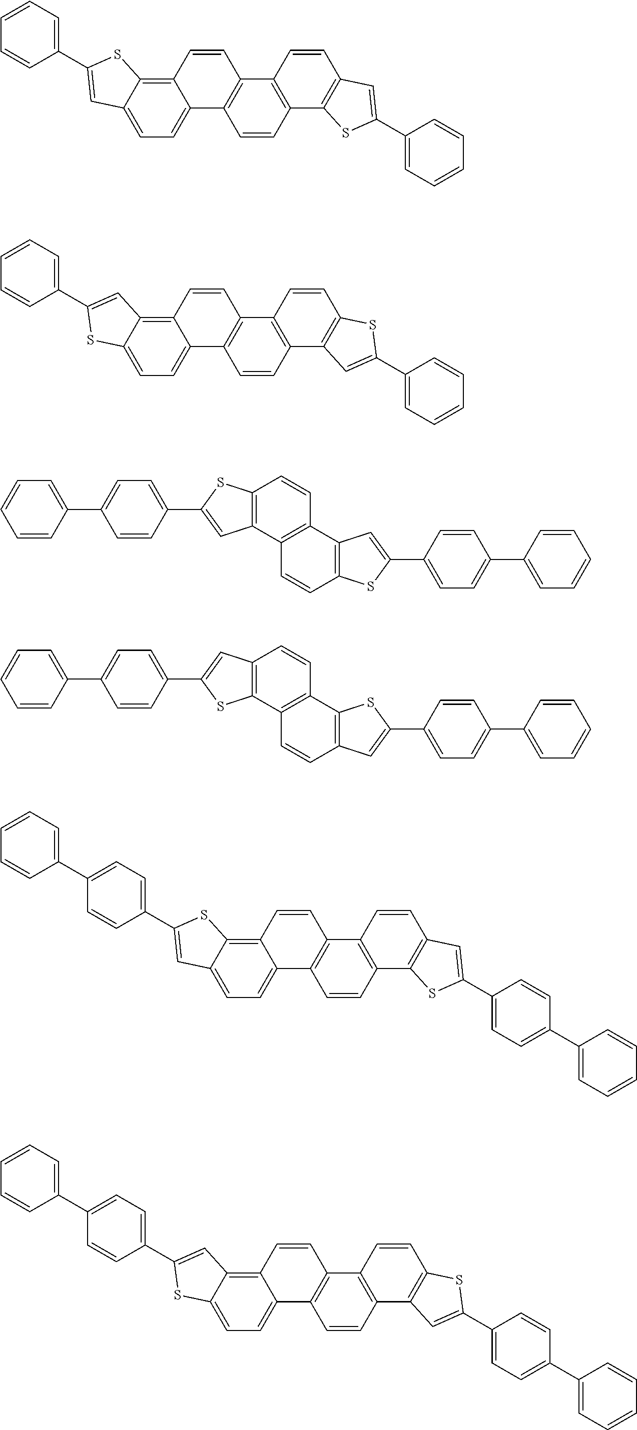 [SMILES: c1ccc(-c2cc3ccc4c5ccc6c(ccc7cc(-c8ccccc8)sc76)c5ccc4c3s2)cc1 ; c1ccc(-c2cc3c(ccc4c3ccc3c5ccc6sc(-c7ccccc7)cc6c5ccc43)s2)cc1 ; c1ccc(-c2ccc(-c3cc4c(ccc5c6cc(-c7ccc(-c8ccccc8)cc7)sc6ccc45)s3)cc2)cc1 ; c1ccc(-c2ccc(-c3cc4ccc5c(ccc6cc(-c7ccc(-c8ccccc8)cc7)sc65)c4s3)cc2)cc1 ; c1ccc(-c2ccc(-c3cc4ccc5c6ccc7c(ccc8cc(-c9ccc(-c%10ccccc%10)cc9)sc87)c6ccc5c4s3)cc2)cc1 ; c1ccc(-c2ccc(-c3cc4c(ccc5c4ccc4c6ccc7sc(-c8ccc(-c9ccccc9)cc8)cc7c6ccc54)s3)cc2)cc1]

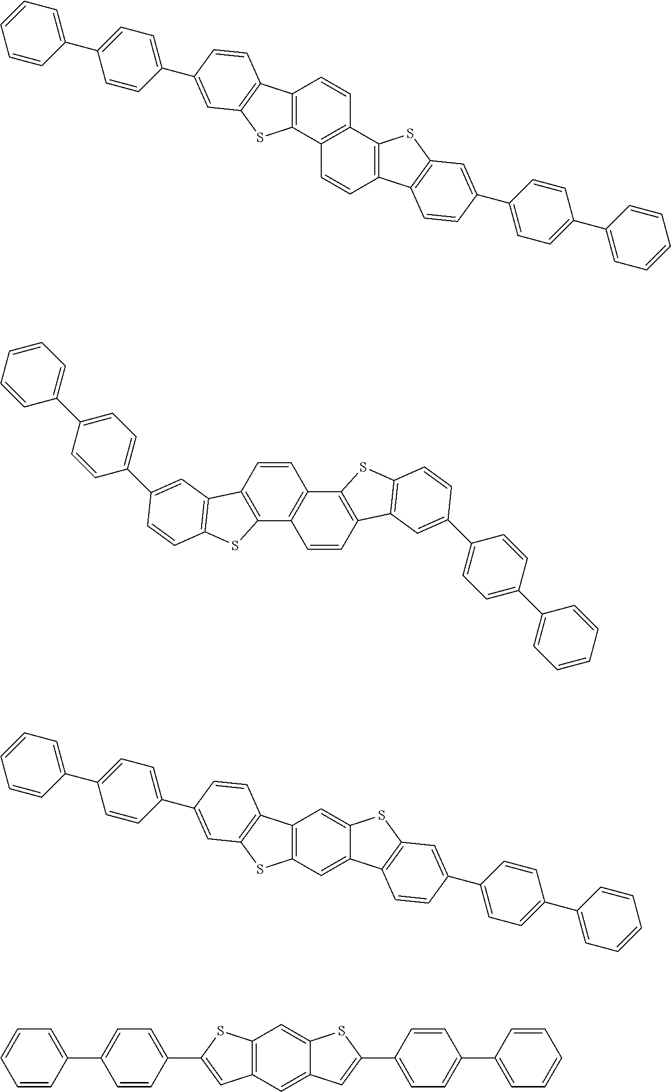 [SMILES: c1ccc(-c2ccc(-c3ccc4c(c3)sc3c4ccc4c3ccc3c5ccc(-c6ccc(-c7ccccc7)cc6)cc5sc34)cc2)cc1 ; c1ccc(-c2ccc(-c3ccc4sc5c(ccc6c5ccc5c7cc(-c8ccc(-c9ccccc9)cc8)ccc7sc56)c4c3)cc2)cc1 ; c1ccc(-c2ccc(-c3ccc4c(c3)sc3cc5c(cc34)sc3cc(-c4ccc(-c6ccccc6)cc4)ccc35)cc2)cc1 ; c1ccc(-c2ccc(-c3cc4cc5cc(-c6ccc(-c7ccccc7)cc6)sc5cc4s3)cc2)cc1]

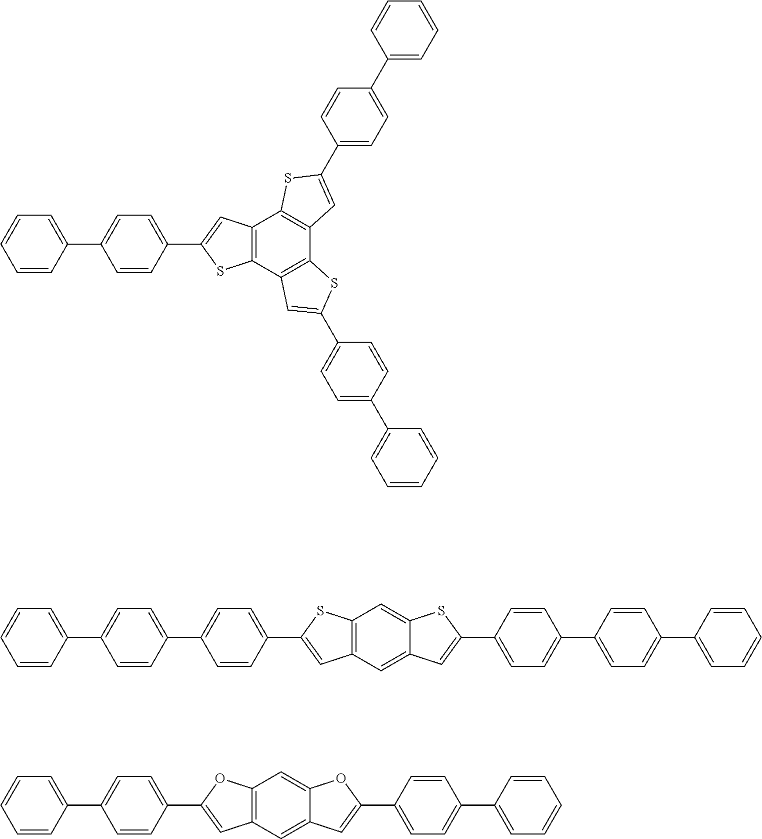 [SMILES: c1ccc(-c2ccc(-c3cc4c(s3)c3cc(-c5ccc(-c6ccccc6)cc5)sc3c3cc(-c5ccc(-c6ccccc6)cc5)sc43)cc2)cc1 ; c1ccc(-c2ccc(-c3ccc(-c4cc5cc6cc(-c7ccc(-c8ccc(-c9ccccc9)cc8)cc7)sc6cc5s4)cc3)cc2)cc1 ; c1ccc(-c2ccc(-c3cc4cc5cc(-c6ccc(-c7ccccc7)cc6)oc5cc4o3)cc2)cc1]

A difference in ionization potential between the specific compound and the p-type organic semiconductor is preferably 0.1 eV or more.

The p-type organic semiconductor material may be used alone or in combination of two or more types thereof.

In a case where the photoelectric conversion film contains the p-type organic semiconductor, a content of the p-type organic semiconductor in the photoelectric conversion film (Film thickness of p-type organic semiconductor in terms of single layer/Film thickness of photoelectric conversion film×100) is preferably 15% to 75% by volume, more preferably 20% to 60% by volume, and still more preferably 25% to 50% by volume.

The photoelectric conversion film containing the specific compound is a non-luminescent film, and has a feature different from an organic light emitting diode (OLED). The non-luminescent film refers to a film having a light emission quantum efficiency of 1% or less, and the light emission quantum efficiency is preferably 0.5% or less and more preferably 0.1% or less. The lower limit thereof is often 0% or more.

### <Coloring Agent>

The photoelectric conversion film preferably contains a coloring agent in addition to the above-described specific compound.

The coloring agent is a compound different from the above-described specific compound.

As the coloring agent, an organic coloring agent is preferable.

Examples of the organic coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a flugide coloring agent, a perylene coloring agent, a phenazine coloring agent, a phenothiazine coloring agent, a quinone coloring agent, a diphenylmethane coloring agent, a polyene coloring agent, an acridine coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, a metal complex, an imidazoquinoxaline coloring agent described in WO2020/013246A, WO2022/168856A, JP2023-10305A, and JP2023-10299A, an acceptor-donor-acceptor type coloring agent in which two acidic nuclei are bonded to a donor, and a donor-acceptor-donor type coloring agent in which two donors are bonded to an acceptor. Among these, from the viewpoint of maximal absorption wavelength, a cyanine coloring agent, an imidazoquinoxaline coloring agent, or an acceptor-donor-acceptor type coloring agent is preferable.

A maximal absorption wavelength of the coloring agent is preferably in the visible light region, more preferably in a wavelength range of 400 to 650 nm, and still more preferably in a wavelength range of 450 to 650 nm.

The coloring agent may be used alone or in combination of two or more types thereof.

A content of the coloring agent with respect to the total content of the specific compound and the coloring agent in the photoelectric conversion film (=(Film thickness of coloring agent in terms of single layer/(Film thickness of specific compound in terms of single layer+Film thickness of coloring agent in terms of single layer)×100)) is preferably 5% to 75% by volume, more preferably 5% to 60% by volume, and still more preferably 5% to 50% by volume.

### <Film Formation Method>

Examples of a film formation method of the above-described photoelectric conversion film include a dry film formation method.

Examples of the dry film formation method include a physical vapor deposition method such as a vapor deposition method (particularly, a vacuum vapor deposition method), a sputtering method, an ion plating method, and a molecular beam epitaxy (MBE) method, and a chemical vapor deposition (CVD) method such as plasma polymerization; and a vacuum vapor deposition method is preferable. In a case where the photoelectric conversion film is formed by the vacuum vapor deposition method, manufacturing conditions such as a degree of vacuum and a vapor deposition temperature can be set according to the conventional method.

A film thickness of the photoelectric conversion film is preferably 10 to 1,000 nm, more preferably 50 to 800 nm, and still more preferably 50 to 500 nm.

### [Electrode]

The photoelectric conversion element preferably includes an electrode.

The electrode (the upper electrode (transparent conductive film) 15 and the lower electrode (conductive film) 11) contains a conductive material. Examples of the conductive material include metals, alloys, metal oxides, electrically conductive compounds, and mixtures thereof.

Since light is incident through the upper electrode 15, the upper electrode 15 is preferably transparent to light to be detected. Examples of a material constituting the upper electrode 15 include conductive metal oxides such as tin oxide doped with antimony, fluorine, or the like (antimony tin oxide (ATO) and fluorine doped tin oxide (FTO)), tin oxide, zinc oxide, indium oxide, indium tin oxide (ITO), and indium zinc oxide (IZO); metal thin films such as gold, silver, chromium, and nickel; mixtures or laminates of these metals and the conductive metal oxides; and organic conductive materials such as polyaniline, polythiophene, and polypyrrole; and nano carbon materials such as carbon nanotubes and graphene. From the viewpoint of high conductivity and transparency, conductive metal oxides are preferable.

In general, in a case where the conductive film is thinner than a certain range, a resistance value rapidly increases in many cases. In a solid-state imaging element in which the photoelectric conversion element according to the present embodiment is incorporated, a sheet resistance may be 100 to 10,000Ω/□, and a degree of freedom of the film thickness range which can be reduced is large.

In addition, as the film thickness of the upper electrode (transparent conductive film) 15 is thinner, the amount of light which is absorbed in the upper electrode is smaller, and thus light transmittance usually increases. The increase in the light transmittance causes an increase in light absorbance in the photoelectric conversion film and an increase in the photoelectric conversion ability, which is preferable. Considering suppression of leakage current, increase in resistance value of the thin film, and increase in transmittance accompanied by the thinning, the thickness of the upper electrode 15 is preferably 5 to 100 nm, and more preferably 5 to 20 nm.

There is a case where the lower electrode 11 has transparency or an opposite case where the lower electrode does not have transparency and reflects light, depending on use. Examples of a material constituting the lower electrode 11 include conductive metal oxides such as tin oxide doped with antimony, fluorine, or the like (ATO and FTO), tin oxide, zinc oxide, indium oxide, indium tin oxide (ITO), and indium zinc oxide (IZO); metals such as gold, silver, chromium, nickel, titanium, tungsten, and aluminum; conductive compounds such as oxides or nitrides of these metals (for example, titanium nitride (TiN)); mixtures or laminates of these metals and conductive metal oxides; organic conductive materials such as polyaniline, polythiophene, and polypyrrole; and carbon materials such as carbon nanotubes and graphene.

A method of forming the electrode can be appropriately selected in accordance with the electrode material. Specific examples thereof include a wet method such as a printing method and a coating method; a physical method such as a vacuum vapor deposition method, a sputtering method, and an ion plating method; and a chemical method such as a CVD method and a plasma CVD method.

In a case where the electrode material is ITO, examples thereof include an electron beam method, a sputtering method, a resistance thermal vapor deposition method, a chemical reaction method (such as a sol-gel method), and a coating method with a dispersion of indium tin oxide.

### [Charge Blocking Film: Electron Blocking Film and Hole Blocking Film]

It is preferable that the photoelectric conversion element includes one or more interlayers between the conductive film and the transparent conductive film, in addition to the photoelectric conversion film.

Examples of the above-described interlayer include a charge blocking film. In a case where the photoelectric conversion element includes the film, the characteristics (such as the quantum efficiency and the response speed) of the photoelectric conversion element to be obtained are more excellent. Examples of the charge blocking film include an electron blocking film and a hole blocking film.

### <Electron Blocking Film>

The electron blocking film is a donor-type organic semiconductor material (compound), and the above-described p-type organic semiconductor can be used.

In addition, a polymer material can also be used in the electron blocking film.

Specific examples of the polymer material include a polymer such as phenylenevinylene, fluorene, carbazole, indole, pyrene, pyrrole, picoline, thiophene, acetylene, and diacetylene, and derivatives thereof.

The electron blocking film may be configured by a plurality of films.

The electron blocking film may be formed of an inorganic material. In general, since an inorganic material has a dielectric constant larger than that of an organic material, in a case where the inorganic material is used in the electron blocking film, a large voltage is applied to the photoelectric conversion film. Therefore, the quantum efficiency increases. Examples of the inorganic material which can be used in the electron blocking film include calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide, and iridium oxide.

### <Hole Blocking Film>

The hole blocking film is an acceptor-type organic semiconductor material (compound), and the above-described n-type organic semiconductor described above can be used.

The hole blocking film may be configured by a plurality of films.

Examples of a method of manufacturing the charge blocking film include a dry film formation method and a wet film formation method. Examples of the dry film formation method include a vapor deposition method and a sputtering method. The vapor deposition method may be a physical vapor deposition (PVD) method or a chemical vapor deposition (CVD) method, and a physical vapor deposition method such as a vacuum vapor deposition method is preferable. Examples of the wet film formation method include an ink jet method, a spray method, a nozzle printing method, a spin coating method, a dip coating method, a casting method, a die coating method, a roll coating method, a bar coating method, and a gravure coating method; and an inkjet method is preferable from the viewpoint of high-precision patterning.

Each film thickness of the charge blocking films (the electron blocking film and the hole blocking film) is preferably 3 to 200 nm, more preferably 5 to 100 nm, and still more preferably 5 to 30 nm.

### [Substrate]

The photoelectric conversion element may further include a substrate.

Examples of the substrate include a semiconductor substrate, a glass substrate, and a plastic substrate.

As a position of the substrate, in general, the conductive film, the photoelectric conversion film, and the transparent conductive film are laminated on the substrate in this order.

### [Sealing Layer]

The photoelectric conversion element may further include a sealing layer.

The performance of the photoelectric conversion material may deteriorate significantly due to the presence of deterioration factors such as water molecules. The deterioration can be prevented by coating and sealing the entirety of the photoelectric conversion film with a sealing layer such as diamond-like carbon (DLC) and ceramics such as metal oxide, metal nitride, or metal nitride oxide, which are dense and into which water molecules do not permeate.

Examples of the sealing layer include sealing layers described in paragraphs [0210] to [0215] of JP2011-082508A, the contents of which are incorporated herein by reference.

### [Imaging Element]

Examples of the application of the photoelectric conversion element include an imaging element.

The imaging element is an element which converts optical information of an image into the electric signal, and is usually an element in which a plurality of photoelectric conversion elements are arranged in a matrix on the same plane, optical signals are converted into electric signals in each photoelectric conversion element (a pixel), and the electric signals can be sequentially output to the outside of the imaging elements for each pixel. Therefore, each pixel is formed of one or more photoelectric conversion elements and one or more transistors.

### [Optical Sensor]

Examples of another application of the photoelectric conversion element include a photoelectric cell and a optical sensor, but it is preferable that the photoelectric conversion element according to the embodiment of the present invention is used as the optical sensor. The above-described photoelectric conversion element may be used alone as the optical sensor, or may be used as a line sensor in which the photoelectric conversion elements are linearly arranged or as a two-dimensional sensor in which the photoelectric conversion elements are arranged on a plane.

### [Compound]

The present invention also includes the specific compound.

### EXAMPLES

Hereinbelow, the present invention will be described in more detail with reference to Examples.

The materials, the amounts of materials used, the proportions, the treatment details, the treatment procedure, and the like shown in Examples below may be modified as appropriate as long as the modifications do not depart from the spirit of the present invention. Therefore, the scope of the present invention should not be construed as being limited to Examples shown below.

### [Compound Used for Photoelectric Conversion Film]

Hereinafter, each material used for a photoelectric conversion film is shown.

### [Synthesis of Compound (1-1)]

A compound (1-1) was synthesized according to the following scheme.

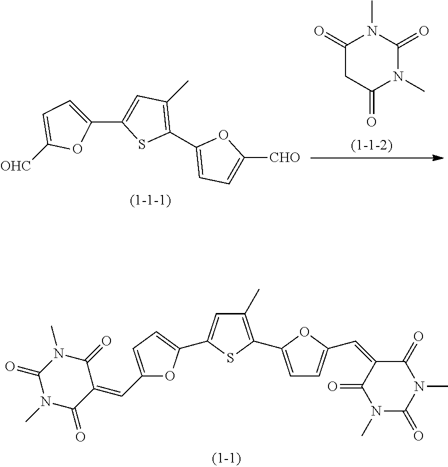 [SMILES: Cc1cc(-c2ccc(C=O)o2)sc1-c1ccc(C=O)o1 ; CN1C(=O)CC(=O)N(C)C1=O ; Cc1cc(-c2ccc(C=C3C(=O)N(C)C(=O)N(C)C3=O)o2)sc1-c1ccc(C=C2C(=O)N(C)C(=O)N(C)C2=O)o1]

A compound (1-1-1) (2.0 mmol), a compound (1-1-2) (5.0 mmol), toluene (60 mL), and piperidine (0.02 mmol) were charged into a glass reaction container, and reacted at 100° C. for 2 hours under a nitrogen atmosphere. The precipitated solid was filtered, and the obtained solid was washed sequentially with tetrahydrofuran (THF), dimethylacetamide (DMAc), and THE, and then sublimated and purified to obtain 1.4 mmol (yield: 70%) of a compound (1-1). A structure of the compound (1-1) was confirmed by LDI-MS.

LDI-MS (Compound (1-1)): 562 (M⁺)

A specific compound used for the photoelectric conversion film, other than the compound (1-1), was synthesized according to the synthesis method of the compound (1-1).

### [Specific Compound]

The specific compounds used for the photoelectric conversion film and comparative compounds of Comparative Examples are shown below.

All of the compounds (1-1) to (1-28), the compounds (2-1) to (2-8), the compounds (3-1) to (3-7), the compounds (4-1) to (4-6), and the compounds (5-1) to (5-9) correspond to the specific compound according to the present invention; and all of the compounds (C-1) to (C-11) are the comparative compounds of Comparative Examples.

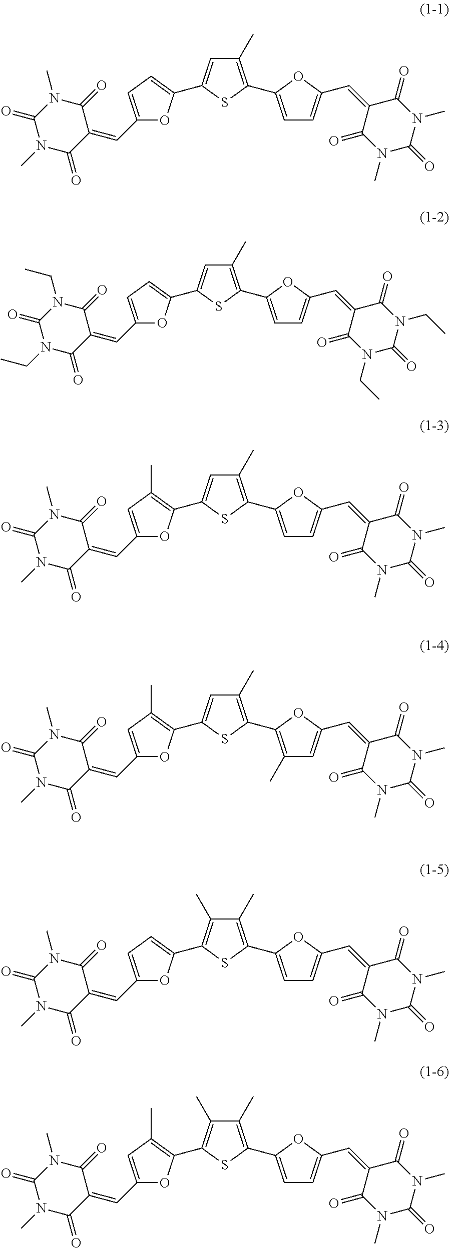

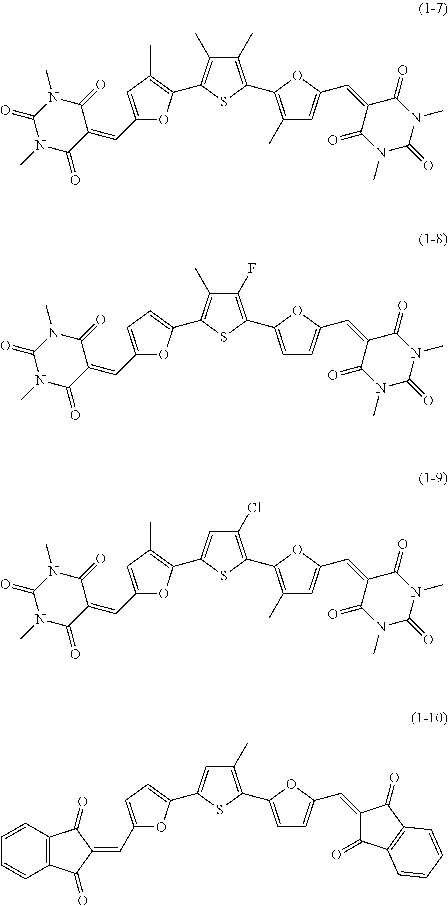 [SMILES: Cc1cc(C=C2C(=O)N(C)C(=O)N(C)C2=O)oc1-c1sc(-c2oc(C=C3C(=O)N(C)C(=O)N(C)C3=O)cc2C)c(C)c1C ; Cc1c(-c2ccc(C=C3C(=O)N(C)C(=O)N(C)C3=O)o2)sc(-c2ccc(C=C3C(=O)N(C)C(=O)N(C)C3=O)o2)c1F ; Cc1cc(C=C2C(=O)N(C)C(=O)N(C)C2=O)oc1-c1cc(Cl)c(-c2oc(C=C3C(=O)N(C)C(=O)N(C)C3=O)cc2C)s1 ; Cc1cc(-c2ccc(C=C3C(=O)c4ccccc4C3=O)o2)sc1-c1ccc(C=C2C(=O)c3ccccc3C2=O)o1]

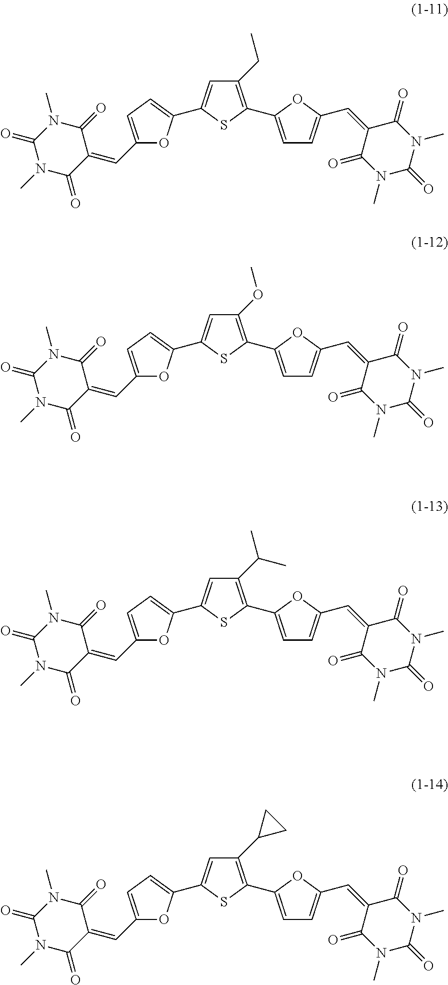 [SMILES: CCc1cc(-c2ccc(C=C3C(=O)N(C)C(=O)N(C)C3=O)o2)sc1-c1ccc(C=C2C(=O)N(C)C(=O)N(C)C2=O)o1 ; COc1cc(-c2ccc(C=C3C(=O)N(C)C(=O)N(C)C3=O)o2)sc1-c1ccc(C=C2C(=O)N(C)C(=O)N(C)C2=O)o1 ; CC(C)c1cc(-c2ccc(C=C3C(=O)N(C)C(=O)N(C)C3=O)o2)sc1-c1ccc(C=C2C(=O)N(C)C(=O)N(C)C2=O)o1 ; CN1C(=O)C(=Cc2ccc(-c3cc(C4CC4)c(-c4ccc(C=C5C(=O)N(C)C(=O)N(C)C5=O)o4)s3)o2)C(=O)N(C)C1=O]

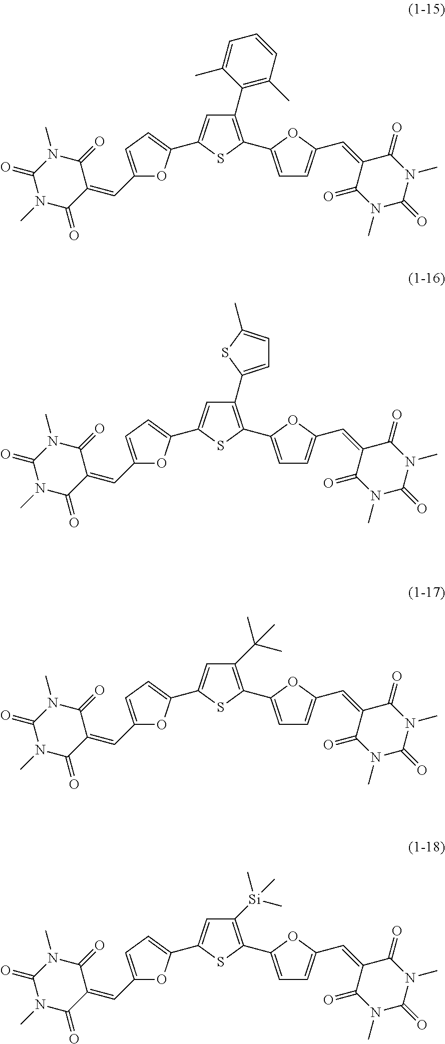 [SMILES: Cc1cccc(C)c1-c1cc(-c2ccc(C=C3C(=O)N(C)C(=O)N(C)C3=O)o2)sc1-c1ccc(C=C2C(=O)N(C)C(=O)N(C)C2=O)o1 ; Cc1ccc(-c2cc(-c3ccc(C=C4C(=O)N(C)C(=O)N(C)C4=O)o3)sc2-c2ccc(C=C3C(=O)N(C)C(=O)N(C)C3=O)o2)s1 ; CN1C(=O)C(=Cc2ccc(-c3cc(C(C)(C)C)c(-c4ccc(C=C5C(=O)N(C)C(=O)N(C)C5=O)o4)s3)o2)C(=O)N(C)C1=O ; CN1C(=O)C(=Cc2ccc(-c3cc([Si](C)(C)C)c(-c4ccc(C=C5C(=O)N(C)C(=O)N(C)C5=O)o4)s3)o2)C(=O)N(C)C1=O]

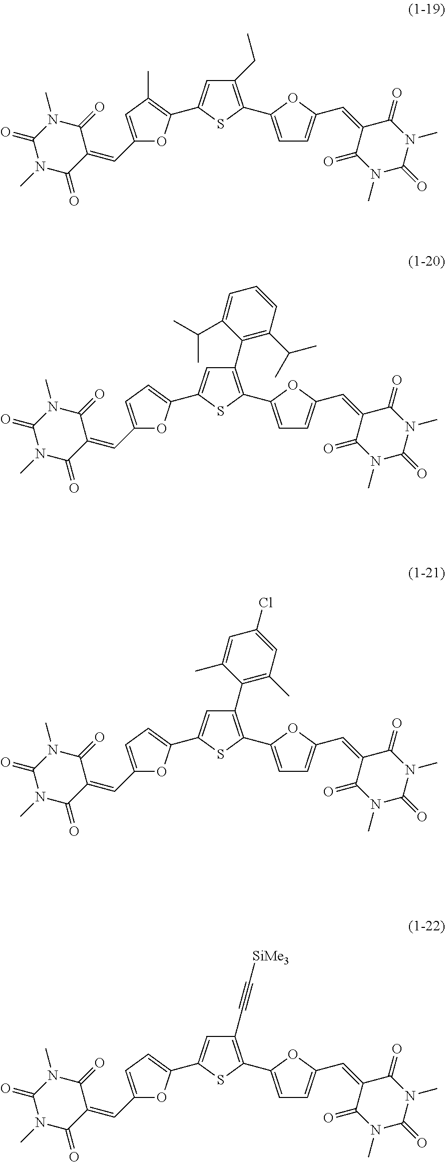 [SMILES: CCc1cc(-c2oc(C=C3C(=O)N(C)C(=O)N(C)C3=O)cc2C)sc1-c1ccc(C=C2C(=O)N(C)C(=O)N(C)C2=O)o1 ; CC(C)c1cccc(C(C)C)c1-c1cc(-c2ccc(C=C3C(=O)N(C)C(=O)N(C)C3=O)o2)sc1-c1ccc(C=C2C(=O)N(C)C(=O)N(C)C2=O)o1 ; Cc1cc(Cl)cc(C)c1-c1cc(-c2ccc(C=C3C(=O)N(C)C(=O)N(C)C3=O)o2)sc1-c1ccc(C=C2C(=O)N(C)C(=O)N(C)C2=O)o1 ; CN1C(=O)C(=Cc2ccc(-c3cc(C#C[Si](C)(C)C)c(-c4ccc(C=C5C(=O)N(C)C(=O)N(C)C5=O)o4)s3)o2)C(=O)N(C)C1=O]

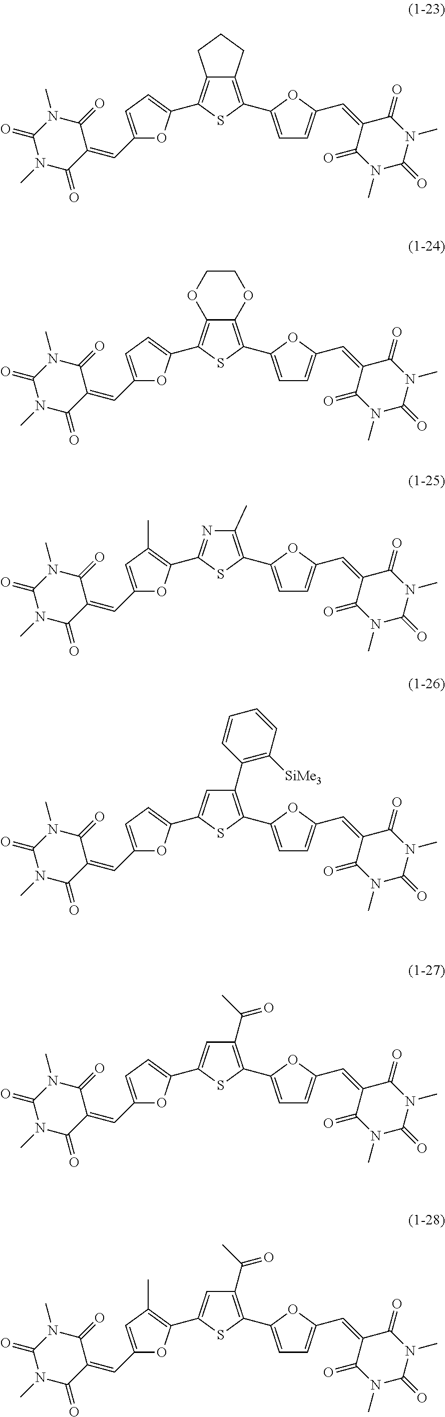

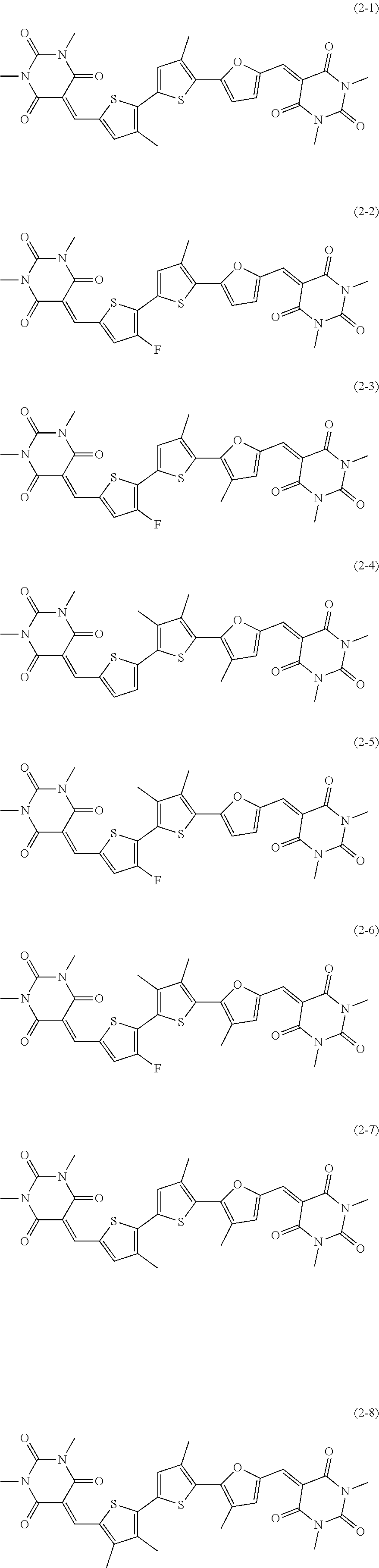

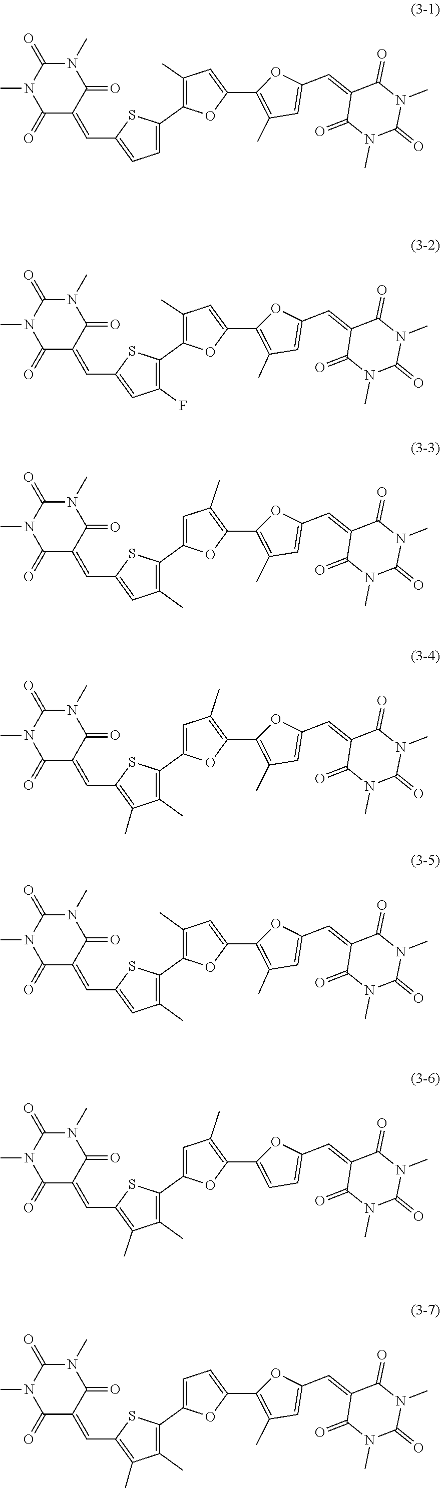

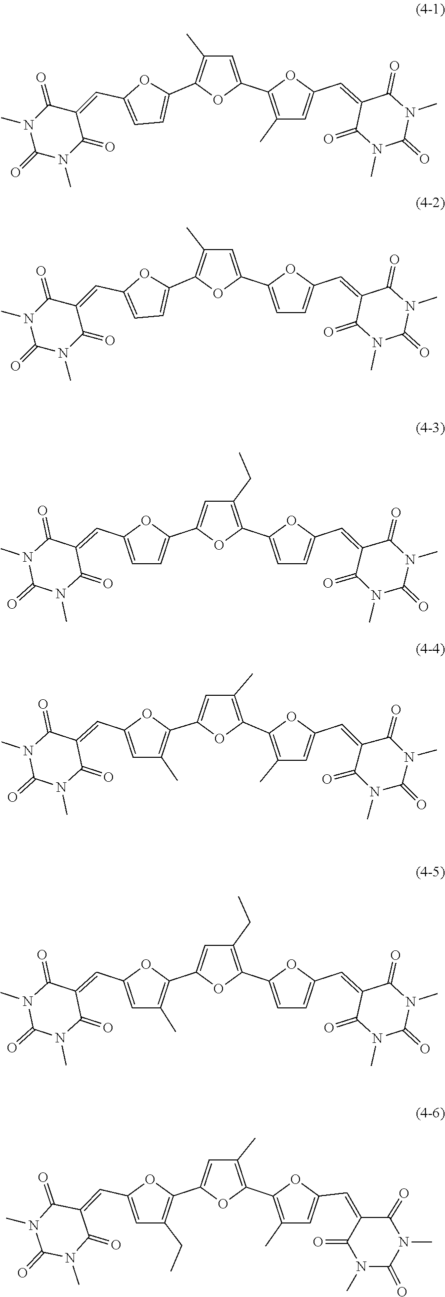

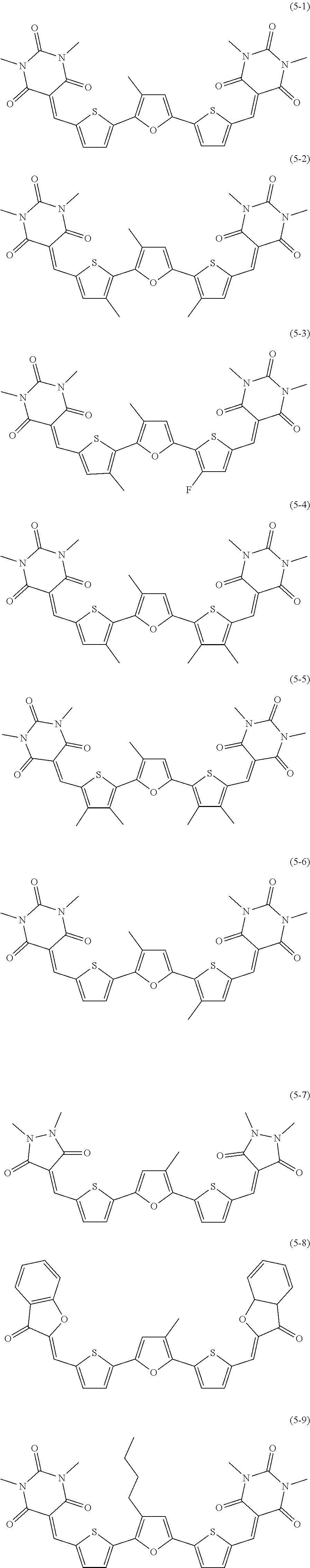

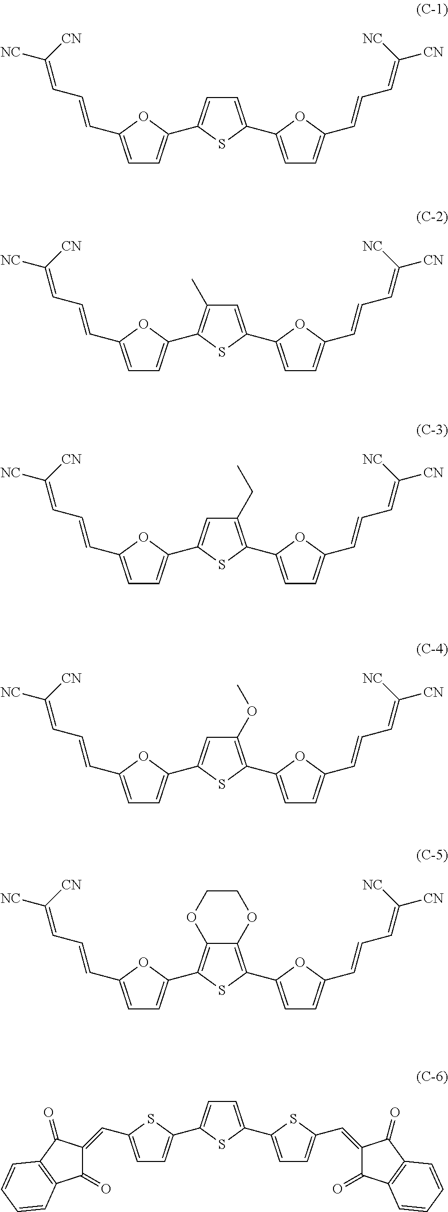 [SMILES: N#CC(C#N)=C/C=C/c1ccc(-c2ccc(-c3ccc(/C=C/C=C(C#N)C#N)o3)s2)o1 ; Cc1cc(-c2ccc(/C=C/C=C(C#N)C#N)o2)sc1-c1ccc(/C=C/C=C(C#N)C#N)o1 ; CCc1cc(-c2ccc(/C=C/C=C(C#N)C#N)o2)sc1-c1ccc(/C=C/C=C(C#N)C#N)o1 ; COc1cc(-c2ccc(/C=C/C=C(C#N)C#N)o2)sc1-c1ccc(/C=C/C=C(C#N)C#N)o1 ; N#CC(C#N)=C/C=C/c1ccc(-c2sc(-c3ccc(/C=C/C=C(C#N)C#N)o3)c3c2OCCO3)o1 ; O=C1C(=Cc2ccc(-c3ccc(-c4ccc(C=C5C(=O)c6ccccc6C5=O)s4)s3)s2)C(=O)c2ccccc21]

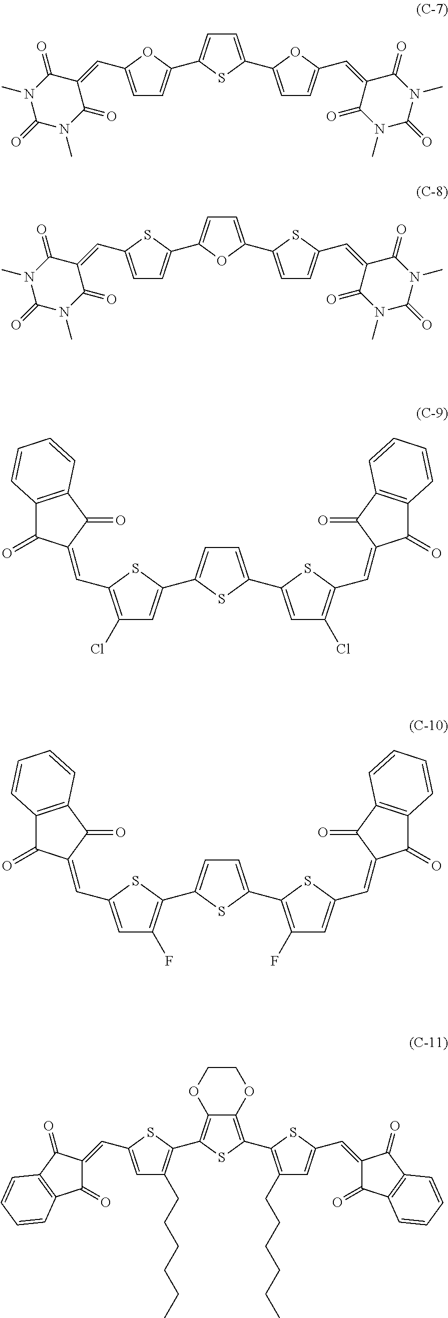

[n-Type Organic Semiconductor]
C60: fullerene (C₆₀)
   [p-Type Organic Semiconductor]

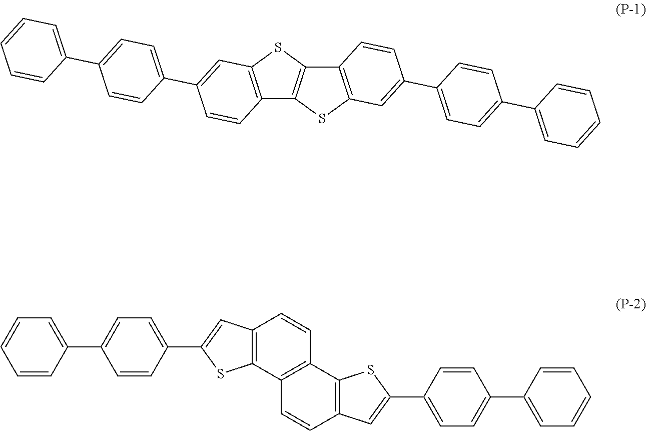 [SMILES: c1ccc(-c2ccc(-c3ccc4c(c3)sc3c5ccc(-c6ccc(-c7ccccc7)cc6)cc5sc43)cc2)cc1 ; c1ccc(-c2ccc(-c3cc4ccc5c(ccc6cc(-c7ccc(-c8ccccc8)cc7)sc65)c4s3)cc2)cc1]

### [Coloring Agent]

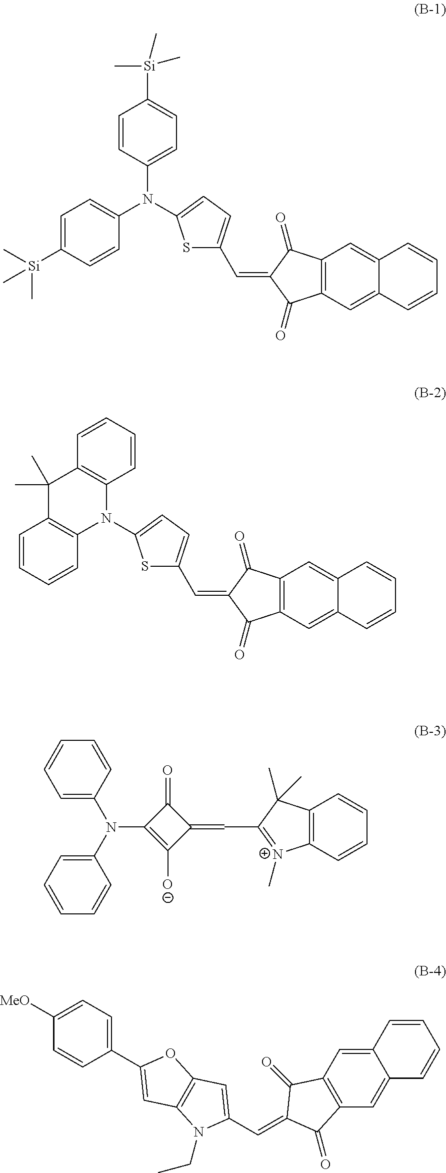 [SMILES: C[Si](C)(C)c1ccc(N(c2ccc([Si](C)(C)C)cc2)c2ccc(C=C3C(=O)c4cc5ccccc5cc4C3=O)s2)cc1 ; CC1(C)c2ccccc2N(c2ccc(C=C3C(=O)c4cc5ccccc5cc4C3=O)s2)c2ccccc21 ; C[N+]1=C(C=C2C(=O)C(N(c3ccccc3)c3ccccc3)=C2[O-])C(C)(C)c2ccccc21 ; CCn1c(C=C2C(=O)c3cc4ccccc4cc3C2=O)cc2oc(-c3ccc(OC)cc3)cc21]

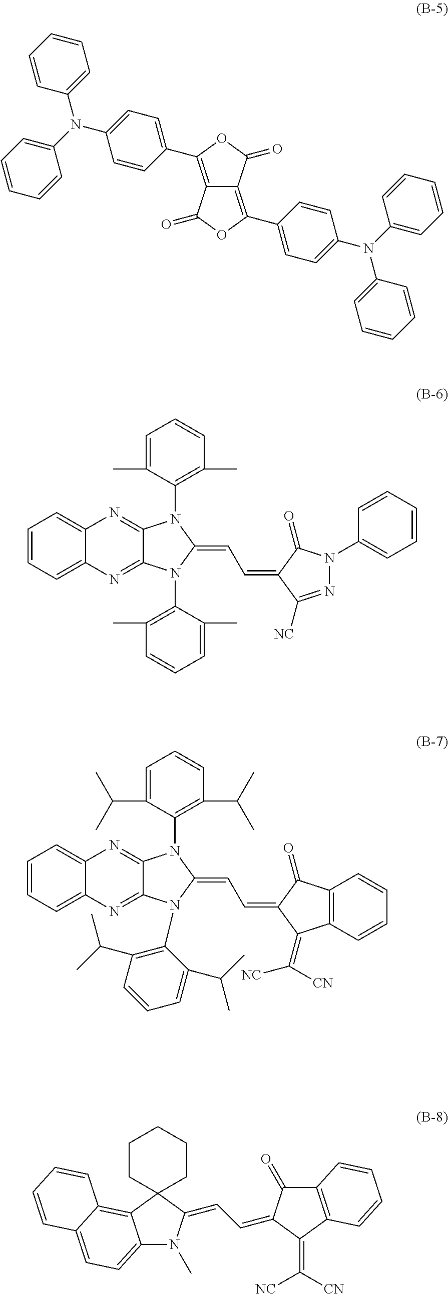 [SMILES: O=C1OC(c2ccc(N(c3ccccc3)c3ccccc3)cc2)=C2C(=O)OC(c3ccc(N(c4ccccc4)c4ccccc4)cc3)=C12 ; Cc1cccc(C)c1N1C(=C/C=C2\C(=O)N(c3ccccc3)N=C2C#N)N(c2c(C)cccc2C)c2nc3ccccc3nc21 ; CC(C)c1cccc(C(C)C)c1N1C(=C/C=C2\C(=O)c3ccccc3C2=C(C#N)C#N)N(c2c(C(C)C)cccc2C(C)C)c2nc3ccccc3nc21 ; CN1/C(=C\C=C2/C(=O)c3ccccc3C2=C(C#N)C#N)C2(CCCCC2)c2c1ccc1ccccc21]

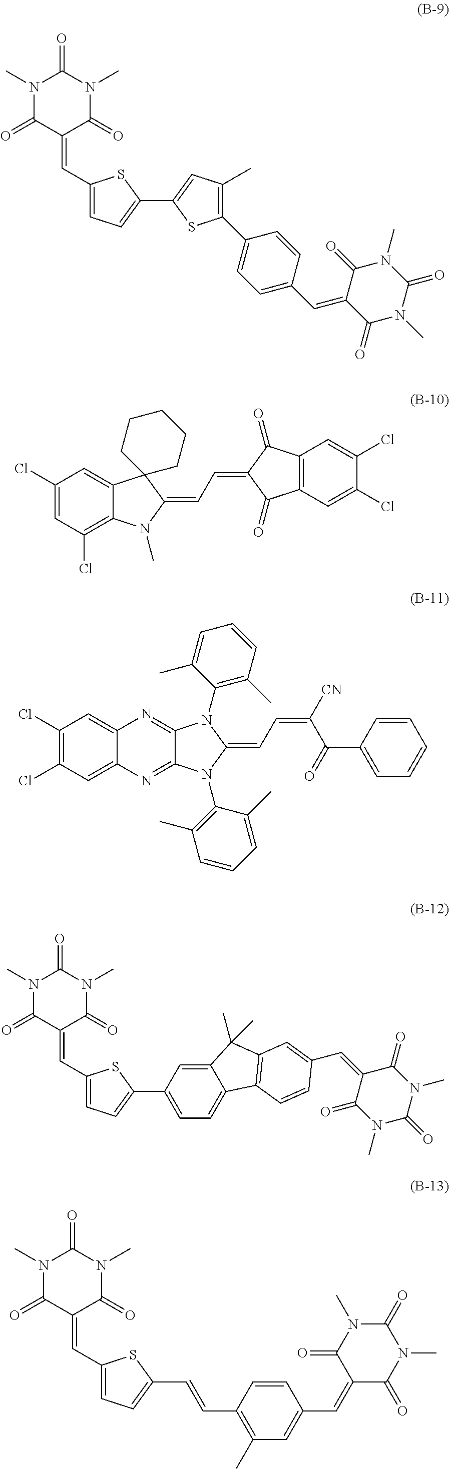

### [Evaluation]

### [Test X]

### <Production of Photoelectric Conversion Element (A)>

A photoelectric conversion element (A) of the form shown in FIG. 2 was produced using the obtained compound. Here, the photoelectric conversion element included a lower electrode 11, an electron blocking film 16A, a photoelectric conversion film 12, a hole blocking film 16B, and an upper electrode 15.

Specifically, an amorphous ITO was formed into a film on a glass substrate by a sputtering method to form the lower electrode 11 (thickness: 30 nm), and the compound (EB-1) was further formed into a film on the lower electrode 11 by a vacuum thermal vapor deposition method to form the electron blocking film 16A (thickness: 30 nm).

Furthermore, in a state in which the temperature of the glass substrate was controlled to 25° C., each specific compound or each comparative compound shown in Table 1 or Table 2, an n-type organic semiconductor (fullerene (C₆₀)), and a p-type organic semiconductor shown in Table 1 or Table 2 were subjected to co-vapor deposition by a vacuum vapor deposition method at a predetermined ratio (specific compound:n-type organic semiconductor:p-type organic semiconductor=1:1:1; in terms of thickness) to form a film on the electron blocking film 16A. As a result, the photoelectric conversion film 12 having a bulk hetero structure with 300 nm was formed. In this case, a film formation rate of the photoelectric conversion film 12 was set to 1.0 Å/sec.

Furthermore, the compound (EB-2) was vapor-deposited on the photoelectric conversion film 12 to form the hole blocking film 16B (thickness: 10 nm). Amorphous ITO was formed into a film on the hole blocking film 16B by a sputtering method to form the upper electrode 15 (transparent conductive film) (film thickness: 10 nm). After the SiO film was formed as a sealing layer on the upper electrode 15 by a vacuum vapor deposition method, an aluminum oxide (Al₂O₃) layer was formed thereon by an atomic layer chemical vapor deposition (ALCVD) method to produce each photoelectric conversion element (A).

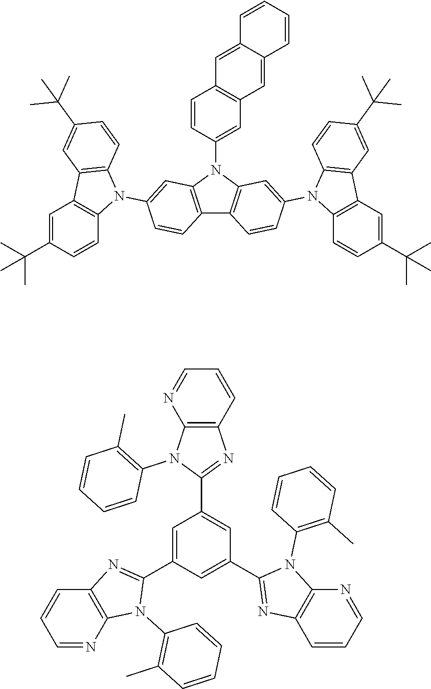 [SMILES: CC(C)(C)c1ccc2c(c1)c1cc(C(C)(C)C)ccc1n2-c1ccc2c3ccc(-n4c5ccc(C(C)(C)C)cc5c5cc(C(C)(C)C)ccc54)cc3n(-c3ccc4cc5ccccc5cc4c3)c2c1 ; Cc1ccccc1-n1c(-c2cc(-c3nc4cccnc4n3-c3ccccc3C)cc(-c3nc4cccnc4n3-c3ccccc3C)c2)nc2cccnc21]

### <Measurement of Dark Current>

A dark current of each obtained photoelectric conversion element (A) was measured by the following method.

A voltage was applied to the lower electrode and the upper electrode of each of the photoelectric conversion elements (A) with an electric field strength of 2.5×10⁵ V/cm, and a current value (dark current) in a dark place was measured. As a result, it was confirmed that all of the photoelectric conversion elements (A) had a dark current of 50 nA/cm² or less, which indicates that all of the photoelectric conversion elements had a sufficiently low dark current.

### <Evaluation of Quantum Efficiency (External Quantum Efficiency)>

Quantum efficiency of each photoelectric conversion element (A) was evaluated by the following method.

A voltage was applied to each photoelectric conversion element (A) with an electric field strength of 2.0×10⁵ V/cm. Thereafter, light was emitted from the upper electrode (transparent conductive film) side, and the measurement of incident photon-to-current conversion efficiency (IPCE) was carried out to extract values of photoelectric conversion efficiency (external quantum efficiency) at a wavelength of 460 nm and a wavelength of 600 nm. The photoelectric conversion efficiency was measured using a constant energy quantum efficiency measuring device manufactured by OPTEL Co., LTD. The amount of light emitted was 50 μW/cm².

Using the obtained photoelectric conversion efficiency of each photoelectric conversion element (A), a relative ratio of the quantum efficiency at each wavelength was calculated according to Expression (S1). From the obtained values, the quantum efficiency was evaluated according to the following evaluation standard.

Relative ratio of quantum efficiency=(Photoelectric conversion efficiency of each photoelectric conversion element(A))/(Photoelectric conversion efficiency of photoelectric conversion element(A) of Comparative Example1-10) Expression (S1):

In the calculation of the relative ratio, the values of the photoelectric conversion efficiency at the same wavelength were used as the numerator and the denominator.

A: relative ratio of the quantum efficiency was 1.4 or more.

B: relative ratio of the quantum efficiency was 1.2 or more and less than 1.4.

C: relative ratio of the quantum efficiency was 1.0 or more and less than 1.2.

D: relative ratio of the quantum efficiency was 0.8 or more and less than 1.0.

E: relative ratio of quantum efficiency was less than 0.8

### <Evaluation of Response Speed>

A response speed of each photoelectric conversion element (A) was evaluated by the following method.

A voltage was applied to each photoelectric conversion element (A) with a strength of 2.0×10⁵ V/cm. Thereafter, a light emitting diode (LED) was turned on for an instant to emit light from the upper electrode (transparent conductive film) side, a photocurrent at a wavelength of 580 nm was measured with an oscilloscope, and a rise time until the signal intensity rose from 0% to 97% was measured. Using the obtained rise time of each photoelectric conversion element (A) at a wavelength of 580 nm, a relative response speed was calculated according to Expression (S2). From the obtained values, the response speed was evaluated according to the following evaluation standard.

Relative response speed=(Rise time of each photoelectric conversion element(A))/(Rise time of photoelectric conversion element(A) of Comparative Example1-10) Expression (S2):

A: relative response speed was less than 0.5.

B: relative response speed was 0.5 or more and less than 1.0.

C: relative response speed was 1.0 or more and less than 1.5.

D: relative response speed was 1.5 or more and less than 2.5.

E: relative response speed was 2.5 or more.

### <Evaluation of Electric Field Strength Dependence of Response Speed>

For each photoelectric conversion element (A), electric field strength dependence of the response speed was evaluated by the following method.

A rise time at an applied voltage of 7.5×10⁴ V/cm was measured by the same procedure as in <Evaluation of response speed>, except that the voltage applied to each photoelectric conversion element (A) was changed to 7.5×10⁴ V/cm.

Using the rise time of each photoelectric conversion element (A) at a wavelength of 580 nm at each applied voltage, a relative ratio of the rise time was calculated according to Expression (S3). From the obtained values, electric field strength dependence of the response speed was evaluated according to the following evaluation standard.

Relative ratio of rise time=(Rise time of each photoelectric conversion element(A) at applied voltage of 7.5×10⁴V/cm)/(Rise time of each photoelectric conversion element(A) at applied voltage of 2.0×10⁵V/cm) Expression (S3):

A: relative ratio of the rise time was less than 2.0.

B: relative ratio of the rise time was 2.0 or more and less than 3.0.

C: relative ratio of the rise time was 3.0 or more and less than 4.0.

D: relative ratio of the rise time was 4.0 or more and less than 5.0.

E: relative ratio of the rise time was 5.0 or more.

### <Evaluation of Manufacturing Suitability>

Manufacturing suitability of the photoelectric conversion element having the configuration of each of Examples and Comparative Examples was evaluated by the following method.

A photoelectric conversion element (B) of each of Examples or Comparative Examples was produced according to the same procedure as that of the photoelectric conversion element (A), except that the film formation rate of the photoelectric conversion film 12 was set to 3.0 Å/sec. Next, a photoelectric conversion efficiency of the obtained photoelectric conversion element (B) was measured by the same method as in <Evaluation of quantum efficiency (external quantum efficiency)>.

Using the measured values of the photoelectric conversion efficiency at 600 nm of the photoelectric conversion element (A) having the same configuration in Examples or Comparative Examples and the photoelectric conversion element (B), a relative ratio B/A of the photoelectric conversion efficiency was calculated according to Expression (S4). From the obtained values, the manufacturing suitability was evaluated according to the following evaluation standard.

As the value of the relative ratio B/A was closer to 1, the characteristics of the photoelectric conversion element were less likely to be deteriorated in a case where the film formation rate was increased, that is, the manufacturing suitability was more excellent.

Relative ratio B/A of photoelectric conversion efficiency=(Photoelectric conversion efficiency of photoelectric conversion element(B))/(Photoelectric conversion efficiency of photoelectric conversion element(A)) Expression (S4):

A: relative ratio B/A was 0.90 or more.

B: relative ratio B/A was 0.85 or more and less than 0.90.

C: relative ratio B/A was 0.80 or more and less than 0.85.

D: relative ratio B/A was 0.75 or more and less than 0.80.

E: relative ratio B/A was less than 0.75.

### <Result>

The evaluation results are shown in Tables 1 and 2 below.

In the column of “Formula (1)” of the tables, a case where the specific compound was the compound represented by Formula (1) is indicated as “A”, and a case other than the case is indicated as “B”.

In the column of “X¹” of the tables, a case where the specific compound was a compound in which X¹ in Formula (1) was an oxygen atom is indicated as “A”, and a case other than the case is indicated as “B”.

In the column of “X¹, X³” of the tables, a case where the specific compound was a compound in which X¹ and X³ in Formula (1) were oxygen atoms is indicated as “A”, and a case other than the case is indicated as “B”.

In the column of “Formula (A-2)” of the tables, a case where the specific compound was a compound in which A¹ and A² in Formula (1) were the group represented by Formula (A-2) is indicated as “A”, and a case other than the case is indicated as “B”.

In the column of “Formula (C-1), Formula (C-2)” of the tables, a case where the specific compound was a compound in which A¹ and A² in Formula (1) were the group represented by Formula (C-1) is indicated as “C-1”, a case where the specific compound was a compound in which A¹ and A² in Formula (1) were the group represented by Formula (C-2) is indicated as “C-2”, and a case other than the case is indicated as “B”.

In the column of “R¹” of the tables, a case where the specific compound was a compound in which R^{Z2} in Formula (1) was a group selected from the substituent group R1 is indicated as “A”, and a case other than the case is indicated as “B”.

In the column of “R²” of the tables, a case where the specific compound was a compound in which R^{Z2} in Formula (1) was a group selected from the substituent group R² is indicated as “A”, and a case other than the case is indicated as “B”.

TABLE 1

| | | | | | | | | | | Quantum | | | | |
|---|---|---|---|---|---|---|---|---|---|---|---|---|---|---|
| | | | | | | | | | | efficiency | | | Electric field strength | |
| | | Formula | | | Formula | Formula (C-1), | | | p-Type organic | 460 | 600 | Response | dependence of response | Manufacturing |
| | Compound | (1) | X¹ | X¹, X³ | (A-2) | Formula (C-2) | R1 | R2 | semiconductor | nm | nm | speed | speed | suitability |
| Example 1-1 | 1-1 | A | A | A | A | C-2 | A | A | P-1 | A | A | A | A | A |
| Example 1-2 | 1-1 | A | A | A | A | C-2 | A | A | P-2 | A | A | A | A | A |
| Example 1-3 | 1-2 | A | A | A | A | C-2 | A | A | P-1 | A | A | A | A | A |
| Example 1-4 | 1-3 | A | A | A | A | C-2 | A | A | P-1 | A | A | A | A | A |
| Example 1-5 | 1-4 | A | A | A | A | C-2 | A | A | P-1 | A | A | A | A | A |
| Example 1-6 | 1-5 | A | A | A | A | C-2 | A | A | P-1 | A | A | A | A | A |
| Example 1-7 | 1-6 | A | A | A | A | C-2 | A | A | P-1 | A | A | A | A | A |
| Example 1-8 | 1-7 | A | A | A | A | C-2 | A | A | P-1 | A | A | A | A | A |
| Example 1-9 | 1-8 | A | A | A | A | C-2 | A | A | P-1 | A | A | A | A | A |
| Example 1-10 | 1-9 | A | A | A | A | C-2 | A | A | P-1 | A | A | A | A | A |
| Example 1-11 | 1-10 | A | A | A | A | C-1 | A | A | P-1 | A | A | A | A | B |
| Example 1-12 | 1-11 | A | A | A | A | C-2 | A | A | P-1 | A | A | A | A | A |
| Example 1-13 | 1-12 | A | A | A | A | C-2 | A | B | P-1 | A | A | A | A | B |
| Example 1-14 | 1-13 | A | A | A | A | C-2 | A | A | P-1 | A | A | A | A | A |
| Example 1-15 | 1-14 | A | A | A | A | C-2 | A | A | P-1 | A | A | A | A | A |
| Example 1-16 | 1-15 | A | A | A | A | C-2 | A | A | P-1 | A | A | A | A | A |
| Example 1-17 | 1-16 | A | A | A | A | C-2 | A | A | P-1 | A | A | A | A | A |
| Example 1-18 | 1-17 | A | A | A | A | C-2 | A | A | P-1 | A | A | A | A | A |
| Example 1-19 | 1-18 | A | A | A | A | C-2 | A | A | P-1 | A | A | A | A | A |
| Example 1-20 | 1-19 | A | A | A | A | C-2 | A | A | P-1 | A | A | A | A | A |
| Example 1-21 | 1-20 | A | A | A | A | C-2 | A | A | P-1 | A | A | A | A | A |
| Example 1-22 | 1-21 | A | A | A | A | C-2 | A | A | P-1 | A | A | A | A | A |
| Example 1-23 | 1-22 | A | A | A | A | C-2 | B | B | P-1 | A | A | A | A | C |
| Example 1-24 | 1-23 | A | A | A | A | C-2 | B | B | P-1 | A | A | A | A | C |
| Example 1-25 | 1-24 | A | A | A | A | C-2 | B | B | p-1 | A | A | A | A | C |
| Example 1-26 | 1-25 | A | A | A | A | C-2 | A | A | P-1 | A | A | A | A | A |
| Example 1-27 | 1-26 | A | A | A | A | C-2 | A | A | P-1 | A | A | A | A | A |
| Example 1-28 | 1-27 | A | A | A | A | C-2 | A | A | P-1 | A | A | A | A | A |
| Example 1-29 | 1-28 | A | A | A | A | C-2 | A | A | P-1 | A | A | A | A | A |
| Example 1-30 | 2-1 | A | A | B | A | C-2 | A | A | P-1 | A | A | A | A | B |
| Example 1-31 | 2-2 | A | A | B | A | C-2 | A | A | P-1 | A | A | A | A | B |
| Example 1-32 | 2-3 | A | A | B | A | C-2 | A | A | P-1 | A | A | A | A | B |
| Example 1-33 | 2-4 | A | A | B | A | C-2 | A | A | P-1 | A | A | A | A | B |
| Example 1-34 | 2-5 | A | A | B | A | C-2 | A | A | P-1 | A | A | A | A | B |
| Example 1-35 | 2-6 | A | A | B | A | C-2 | A | A | P-1 | A | A | A | A | B |
| Example 1-36 | 2-7 | A | A | B | A | C-2 | A | A | P-1 | A | A | A | A | B |
| Example 1-37 | 2-8 | A | A | B | A | C-2 | A | A | P-1 | A | A | A | A | B |

TABLE 2

| | | | | | | | | | | Quantum | | | | |
|---|---|---|---|---|---|---|---|---|---|---|---|---|---|---|
| | | | | | | | | | | efficiency | | | Electric field strength | |
| | | Formula | | | Formula | Formula (C-1), | | | p-Type organic | 460 | 600 | Response | dependence of response | Manufacturing |
| | Compound | (1) | X¹ | X¹, X³ | (A-2) | Formula (C-2) | R1 | R2 | semiconductor | nm | nm | speed | speed | suitability |
| Example 1-38 | 3-1 | A | A | B | A | C-2 | A | A | P-1 | A | A | A | A | B |
| Example 1-39 | 3-2 | A | A | B | A | C-2 | A | A | P-1 | A | A | A | A | B |
| Example 1-40 | 3-3 | A | A | B | A | C-2 | A | A | P-1 | A | A | A | A | B |
| Example 1-41 | 3-4 | A | A | B | A | C-2 | A | A | P-1 | A | A | A | A | B |
| Example 1-42 | 3-5 | A | A | B | A | C-2 | A | A | P-1 | A | A | A | A | B |
| Example 1-43 | 3-6 | A | A | B | A | C-2 | A | A | P-1 | A | A | A | A | B |
| Example 1-44 | 3-7 | A | A | B | A | C-2 | A | A | P-1 | A | A | A | A | B |
| Example 1-45 | 4-1 | A | A | A | A | C-2 | A | A | P-1 | A | A | A | A | A |
| Example 1-46 | 4-2 | A | A | A | A | C-2 | A | A | P-1 | A | A | A | A | A |
| Example 1-47 | 4-3 | A | A | A | A | C-2 | A | A | P-1 | A | A | A | A | A |
| Example 1-48 | 4-4 | A | A | A | A | C-2 | A | A | P-1 | A | A | A | A | A |
| Example 1-49 | 4-5 | A | A | A | A | C-2 | A | A | P-1 | A | A | A | A | A |
| Example 1-50 | 4-6 | A | A | A | A | C-2 | A | A | P-1 | A | A | A | A | A |
| Example 1-51 | 5-1 | A | B | B | A | C-2 | A | A | P-1 | A | A | A | A | C |
| Example 1-52 | 5-2 | A | B | B | A | C-2 | A | A | P-1 | A | A | A | A | C |
| Example 1-53 | 5-3 | A | B | B | A | C-2 | A | A | P-1 | A | A | A | A | C |
| Example 1-54 | 5-4 | A | B | B | A | C-2 | A | A | P-1 | A | A | A | A | C |
| Example 1-55 | 5-5 | A | B | B | A | C-2 | A | A | P-1 | A | A | A | A | C |
| Example 1-56 | 5-6 | A | B | B | A | C-2 | A | A | P-1 | A | A | A | A | C |
| Example 1-57 | 5-7 | A | B | B | A | B | A | A | P-1 | B | B | A | A | C |
| Example 1-58 | 5-8 | A | B | B | B | B | A | A | P-1 | B | B | B | B | C |
| Example 1-59 | 5-9 | A | B | B | A | C-2 | B | B | P-1 | B | B | B | B | D |
| Comparative | C-1 | B | A | A | B | B | B | B | P-1 | D | D | E | E | E |
| Example 1-1 | | | | | | | | | | | | | | |
| Comparative | C-2 | B | A | A | B | B | A | A | P-1 | B | B | E | E | E |
| Example 1-2 | | | | | | | | | | | | | | |
| Comparative | C-3 | B | A | A | B | B | A | A | P-1 | B | B | E | E | E |
| Example 1-3 | | | | | | | | | | | | | | |
| Comparative | C-4 | B | A | A | B | B | A | B | P-1 | B | B | E | E | E |
| Example 1-4 | | | | | | | | | | | | | | |
| Comparative | C-5 | B | A | A | B | B | A | B | P-1 | B | B | E | E | E |
| Example 1-5 | | | | | | | | | | | | | | |
| Comparative | C-6 | B | B | B | A | C-1 | B | B | P-1 | D | D | D | D | E |
| Example 1-6 | | | | | | | | | | | | | | |
| Comparative | C-7 | B | A | A | A | C-2 | B | B | P-1 | D | D | D | D | E |
| Example 1-7 | | | | | | | | | | | | | | |
| Comparative | C-8 | B | B | B | A | C-2 | B | B | P-1 | D | D | D | D | E |
| Example 1-8 | | | | | | | | | | | | | | |
| Comparative | C-9 | B | B | B | A | C-1 | A | A | P-1 | E | E | D | D | E |
| Example 1-9 | | | | | | | | | | | | | | |
| Comparative | C-10 | B | B | B | A | C-1 | A | A | P-1 | C | C | C | D | E |
| Example 1-10 | | | | | | | | | | | | | | |
| Comparative | C-11 | B | B | B | A | C-1 | B | B | P-1 | E | E | D | D | E |
| Example 1-11 | | | | | | | | | | | | | | |

From the results shown in Tables 1 and 2, it was found that the photoelectric conversion element according to the embodiment of the present invention had excellent quantum efficiency and excellent manufacturing suitability. In addition, it was found that the photoelectric conversion element according to the embodiment of the present invention also had excellent response speed and electric field strength dependence of the response speed.

From the comparison of Examples 1-30 to 1-44, Examples 1-51 to 1-59, and other examples, it was found that, in a case where X¹ in Formula (1) was an oxygen atom, the manufacturing suitability was more excellent, and in a case where X¹ and X³ were oxygen atoms, the manufacturing suitability was further excellent.

From the comparison of Examples 1-57 and 1-58 and Examples 1-51 to 1-56, it was found that, in a case where A¹ and A² in Formula (1) were the group represented by Formula (A-2), the response speed and the electric field strength dependence of the response speed were more excellent, and in a case where A¹ and A² were the group represented by Formula (C-1) or the group represented by Formula (C-2), the quantum efficiency was more excellent.

From the comparison of Example 1-11 and Examples 1-1 to 1-10, it was found that, in a case where A¹ and A² in Formula (1) were the group represented by Formula (C-2), the manufacturing suitability was more excellent.

From the comparison of Examples 1-13, 1-23 to 1-25, and 1-59, and other examples, it was found that, in a case where R^{Z2} in Formula (1) was a group selected from the above-described substituent group R1, the quantum efficiency, the response speed, the electric field strength dependence of the response speed, and the manufacturing suitability were more excellent, and in a case where R^{Z2} was a group selected from the substituent group R², the manufacturing suitability was further excellent.

### [Test Y]

### <Production of Photoelectric Conversion Element (C)>

A photoelectric conversion element (C) of each of Examples and Comparative Examples was produced in the same manner as in Test X, except that each specific compound or each comparative compound shown in Table 3, C60 as the n-type organic semiconductor, a compound P-1 as the p-type organic semiconductor, and a compound shown in Table 3 as a coloring agent were subjected to co-vapor deposition by a vacuum vapor deposition method at a predetermined ratio (specific compound:coloring agent:p-type organic semiconductor:n-type organic semiconductor=1:1:2:2; in terms of thickness) to form a film, thereby forming the photoelectric conversion film 12 having a thickness of 300 nm.

### <Dark Current>

The dark current of the obtained photoelectric conversion element (C) was measured according to the same procedure as in <Measurement of dark current> in [Test X].

As a result, it was confirmed that all of the photoelectric conversion elements (C) had a dark current of 50 nA/cm² or less, which indicates that all of the photoelectric conversion elements had a sufficiently low dark current.

### <Evaluation of Quantum Efficiency (External Quantum Efficiency)>

The photoelectric conversion efficiency of the obtained photoelectric conversion element (C) at a wavelength of 460 nm and a wavelength of 600 nm was calculated in the same procedure as in <Evaluation of quantum efficiency (external quantum efficiency)> in [Test X].

Using the photoelectric conversion efficiency of each photoelectric conversion element (C), a relative ratio of the quantum efficiency at each wavelength was calculated according to Expression (S5). From the obtained values, the quantum efficiency was evaluated according to the following evaluation standard.

Relative ratio of quantum efficiency=(Photoelectric conversion efficiency of each photoelectric conversion element(C))/(Photoelectric conversion efficiency of photoelectric conversion element(C) of Comparative Example2-10)Expression(S5):

AA: relative ratio of the quantum efficiency was 1.6 or more

A: relative ratio of the quantum efficiency was 1.4 or more and less than 1.6.

B: relative ratio of the quantum efficiency was 1.2 or more and less than 1.4.

C: relative ratio of the quantum efficiency was 1.0 or more and less than 1.2.

D: relative ratio of the quantum efficiency was 0.8 or more and less than 1.0.

E: relative ratio of quantum efficiency was less than 0.8

### <Evaluation of Response Speed>

The rise time of the obtained photoelectric conversion element (C) at a wavelength of 580 nm was measured according to the same procedure as in <Evaluation of response speed> in [Test X].

Using the obtained rise time of each photoelectric conversion element (C) at a wavelength of 580 nm, a relative response speed was calculated according to Expression (S6). From the obtained values, the response speed was evaluated according to the following evaluation standard.

Relative response speed=(Rise time of each photoelectric conversion element(C))/(Rise time of photoelectric conversion element(C) of Comparative Example2-10) Expression (S6):

A: relative response speed was less than 0.5.

B: relative response speed was 0.5 or more and less than 1.0.

C: relative response speed was 1.0 or more and less than 1.5.

D: relative response speed was 1.5 or more and less than 2.5.

E: relative response speed was 2.5 or more.

### <Evaluation of Electric Field Strength Dependence of Response Speed>

For each photoelectric conversion element (C), electric field strength dependence of the response speed was evaluated by the following method.

The rise time of each photoelectric conversion element (C) at an applied voltage of 7.5×10⁴ V/cm and an applied voltage of 2.0×10⁵ V/cm was measured in the same procedure as in <Evaluation of electric field strength dependence of response speed> in [Test Y].

Using the rise time of each photoelectric conversion element (C) at a wavelength of 580 nm at each applied voltage, a relative ratio of the rise time was calculated according to Expression (S7). From the obtained values, electric field strength dependence of the response speed was evaluated according to the following evaluation standard.

Relative ratio of rise time=(Rise time of each photoelectric conversion element(C) at applied voltage of 7.5×10⁴V/cm)/(Rise time of each photoelectric conversion element(C) at applied voltage of 2.0×10⁵V/cm) Expression (S7):

A: relative ratio of the rise time was less than 2.0.

B: relative ratio of the rise time was 2.0 or more and less than 3.0.

C: relative ratio of the rise time was 3.0 or more and less than 4.0.

D: relative ratio of the rise time was 4.0 or more and less than 5.0.

E: relative ratio of the rise time was 5.0 or more.

### <Evaluation of Manufacturing Suitability>

Manufacturing suitability of the photoelectric conversion element having the configuration of each of Examples and Comparative Examples was evaluated by the following method.

A photoelectric conversion element (D) of each of Examples or Comparative Examples was produced according to the same procedure as that of the photoelectric conversion element (C), except that the film formation rate of the photoelectric conversion film 12 was set to 3.0 Å/sec. Next, a photoelectric conversion efficiency of the obtained photoelectric conversion element (D) was measured by the same method as in <Evaluation of quantum efficiency (external quantum efficiency)>.

Using the measured values of the photoelectric conversion efficiency at 600 nm of the photoelectric conversion element (C) having the same configuration in Examples or Comparative Examples and the photoelectric conversion element (D), a relative ratio D/C of the photoelectric conversion efficiency was calculated according to Expression (S8). From the obtained values, the manufacturing suitability was evaluated according to the following evaluation standard.

As the value of the relative ratio D/C was closer to 1, the characteristics of the photoelectric conversion element were less likely to be deteriorated in a case where the film formation rate was increased, that is, the manufacturing suitability was more excellent.

Relative ratio D/C of photoelectric conversion efficiency=(Photoelectric conversion efficiency of photoelectric conversion element(D))/(Photoelectric conversion efficiency of photoelectric conversion element(C)) Expression (S8):

A: relative ratio D/C was 0.90 or more.

B: relative ratio D/C was 0.85 or more and less than 0.90.

C: relative ratio D/C was 0.80 or more and less than 0.85.

D: relative ratio D/C was 0.75 or more and less than 0.80.

E: relative ratio D/C was less than 0.75.

### <Result>

The evaluation results are shown in Table 3 below.

In Table 3, the columns of “Formula (1)”, “X¹”, “X¹, X³”, “Formula (A-2)”, “Formula (C-1), Formula (C-2)”, “R¹”, and “R²” are the same as those in Tables 1 and 2.

TABLE 3

| | | | | | | | | | | Quantum | | | | |
|---|---|---|---|---|---|---|---|---|---|---|---|---|---|---|
| | | | | | | | | | | efficiency | | | Electric field strength | |
| | | Formula | | | Formula | Formula (C-1), | | | Coloring | 460 | 600 | Response | dependence of response | Manufacturing |
| | Compound | (1) | X¹ | X¹, X³ | (A-2) | Formula (C-2) | R1 | R2 | agent | nm | nm | speed | speed | suitability |
| Example 2-1 | 1-1 | A | A | A | A | C-2 | A | A | B-1 | AA | AA | A | A | A |
| Example 2-2 | 1-3 | A | A | A | A | C-2 | A | A | B-2 | AA | AA | A | A | A |
| Example 2-3 | 1-4 | A | A | A | A | C-2 | A | A | B-3 | AA | AA | A | A | A |
| Example 2-4 | 1-8 | A | A | A | A | C-2 | A | A | B-9 | AA | AA | A | A | A |
| Example 2-5 | 1-14 | A | A | A | A | C-2 | A | A | B-2 | AA | AA | A | A | A |
| Example 2-6 | 1-15 | A | A | A | A | C-2 | A | A | B-4 | AA | AA | A | A | A |
| Example 2-7 | 2-1 | A | A | B | A | C-2 | A | A | B-7 | AA | AA | A | A | B |
| Example 2-8 | 2-2 | A | A | B | A | C-2 | A | A | B-13 | AA | AA | A | A | B |
| Example 2-9 | 2-3 | A | A | B | A | C-2 | A | A | B-12 | AA | AA | A | A | B |
| Example 2-10 | 2-7 | A | A | B | A | C-2 | A | A | B-6 | AA | AA | A | A | B |
| Example 2-11 | 3-1 | A | A | B | A | C-2 | A | A | B-5 | AA | AA | A | A | B |
| Example 2-12 | 3-2 | A | A | B | A | C-2 | A | A | B-8 | AA | AA | A | A | B |
| Example 2-13 | 3-3 | A | A | B | A | C-2 | A | A | B-9 | AA | AA | A | A | B |
| Example 2-14 | 3-5 | A | A | B | A | C-2 | A | A | B-10 | AA | AA | A | A | B |
| Example 2-15 | 4-1 | A | A | A | A | C-2 | A | A | B-10 | AA | AA | A | A | A |
| Example 2-16 | 4-2 | A | A | A | A | C-2 | A | A | B-11 | AA | AA | A | A | A |
| Example 2-17 | 4-3 | A | A | A | A | C-2 | A | A | B-9 | AA | AA | A | A | A |
| Example 2-18 | 4-4 | A | A | A | A | C-2 | A | A | B-7 | AA | AA | A | A | A |
| Example 2-19 | 4-5 | A | A | A | A | C-2 | A | A | B-6 | AA | AA | A | A | A |
| Example 2-20 | 5-1 | A | B | B | A | C-2 | A | A | B-5 | AA | AA | A | A | C |
| Example 2-21 | 5-2 | A | B | B | A | C-2 | A | A | B-4 | AA | AA | A | A | C |
| Example 2-22 | 5-3 | A | B | B | A | C-2 | A | A | B-1 | AA | AA | A | A | C |
| Example 2-23 | 5-4 | A | B | B | A | C-2 | A | A | B-8 | AA | AA | A | A | C |
| Example 2-24 | 5-5 | A | B | B | A | C-2 | A | A | B-6 | AA | AA | A | A | C |
| Example 2-25 | 5-6 | A | B | B | A | C-2 | A | A | B-9 | AA | AA | A | A | C |
| Example 2-26 | 5-7 | A | B | B | A | B | A | A | B-10 | A | A | A | A | C |
| Example 2-27 | 5-8 | A | B | B | B | B | A | A | B-11 | A | A | B | B | C |
| Example 2-28 | 5-9 | A | B | B | A | C-2 | B | B | B-12 | A | A | B | B | D |
| Comparative | C-1 | B | A | A | B | B | B | B | B-1 | C | C | E | E | E |
| Example 2-1 | | | | | | | | | | | | | | |
| Comparative | C-2 | B | A | A | B | B | A | A | B-3 | B | B | E | E | E |
| Example 2-2 | | | | | | | | | | | | | | |
| Comparative | C-3 | B | A | A | B | B | A | A | B-13 | B | B | E | E | E |
| Example 2-3 | | | | | | | | | | | | | | |
| Comparative | C-4 | B | A | A | B | B | A | B | B-14 | B | B | E | E | E |
| Example 2-4 | | | | | | | | | | | | | | |
| Comparative | C-5 | B | A | A | B | B | A | B | B-12 | B | B | E | E | E |
| Example 2-5 | | | | | | | | | | | | | | |
| Comparative | C-6 | B | B | B | A | C-1 | B | B | B-5 | C | C | D | D | E |
| Example 2-6 | | | | | | | | | | | | | | |
| Comparative | C-7 | B | A | A | A | C-2 | B | B | B-6 | C | C | D | D | E |
| Example 2-7 | | | | | | | | | | | | | | |
| Comparative | C-8 | B | B | B | A | C-2 | B | B | B-12 | C | C | D | D | E |
| Example 2-8 | | | | | | | | | | | | | | |
| Comparative | C-9 | B | B | B | A | C-1 | A | A | B-10 | E | E | D | D | E |
| Example 2-9 | | | | | | | | | | | | | | |
| Comparative | C-10 | B | B | B | A | C-1 | A | A | B-8 | C | C | C | D | E |
| Example 2-10 | | | | | | | | | | | | | | |
| Comparative | C-11 | B | B | B | A | C-1 | B | B | B-7 | E | E | D | D | E |
| Example 2-11 | | | | | | | | | | | | | | |

From the results shown in Table 3, it was found that the photoelectric conversion element having the configuration of the test Y according to the present invention had excellent effects of the present invention, the response speed and the electric field strength dependence of the response speed with respect to light having a wavelength of 460 nm or 600 nm.

From the comparison of Examples 2-7 to 2-14 and 2-20 to 2-28, and other examples, it was found that, in a case where X¹ in Formula (1) was an oxygen atom, the manufacturing suitability was more excellent, and in a case where X¹ and X³ were oxygen atoms, the manufacturing suitability was further excellent.

From the comparison of Examples 2-26 and 2-27 and Examples 2-20 to 2-25, it was found that, in a case where A¹ and A² in Formula (1) were the group represented by Formula (A-2), the response speed and the electric field strength dependence of the response speed were more excellent, and in a case where A¹ and A² were the group represented by Formula (C-1) or the group represented by Formula (C-2), the quantum efficiency was more excellent.

From the comparison of Example 2-28 and Examples 2-20 to 2-25, it was found that, in a case where R^{Z2} was a group selected from the above-described substituent group R1, the quantum efficiency, the response speed, the electric field strength dependence of the response speed, and the manufacturing suitability were more excellent.

### EXPLANATION OF REFERENCES

10a, 10b: photoelectric conversion element
11: conductive film (lower electrode)
12: photoelectric conversion film
15: transparent conductive film (upper electrode)
16A: electron blocking film
16B: hole blocking film

What is claimed is:

1. A photoelectric conversion element comprising, in the following order:
a conductive film;
a photoelectric conversion film; and
a transparent conductive film,
wherein the photoelectric conversion film contains a compound represented by Formula (1), 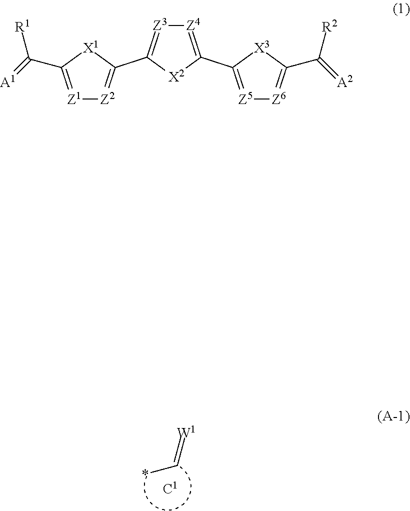
in Formula (1), R¹ and R² each independently represent a hydrogen atom or a substituent,
X¹ to X³ each independently represent a sulfur atom, an oxygen atom, a selenium atom, or a tellurium atom, where at least one of X¹, X², or X³ is an oxygen atom,
Z¹ to Z⁶ each independently represent —CR^{Z1}═ or —N═, R^{Z1} represents a hydrogen atom or a substituent, where at least one of Z¹, . . . , or Z⁶ is —CR^{Z2}═, R^{Z2} represents an aliphatic hydrocarbon group which may have a substituent, an acyl group which may have a substituent, an aromatic ring group which may have a substituent, an aliphatic heterocyclic group which may have a substituent, or a group represented by —Si(R^{Si})₃,
the aliphatic hydrocarbon group represented by R^{Z2} may have a halogen atom and may have an etheric oxygen atom, the acyl group represented by R^{Z2} may have a halogen atom,
R^{Si}'s each independently represent an aliphatic hydrocarbon group which may have a substituent or an aromatic ring group which may have a substituent,
the aliphatic hydrocarbon group represented by R^{Si} may have a halogen atom and may have an etheric oxygen atom,
in a case where Z¹ and Z² are —CR^{Z2}═, two R^{Z2}'s may be bonded to each other to form a ring, in a case where Z³ and Z⁴ are —CR^{Z2}═, two R^{Z2}'s may be bonded to each other to form a ring, and in a case where Z⁵ and Z⁶ are —CR^{Z2}═, two R^{Z2}'s may be bonded to each other to form a ring, and
A¹ and A² each independently represent a group represented by Formula (A-1),
in Formula (A-1), C¹ represents a ring which contains at least two carbon atoms and may have a substituent,
W¹ represents an oxygen atom, a sulfur atom, ═NR^{W1}, or ═CR^{W2}R^{W3}, R^{W1} represents a hydrogen atom or a substituent, R^{W2} and R^{W3} each independently represent a cyano group, —SO₂R^{W4}, —COOR^{W5}, or —COR^{W6}, R^{W4}, R^{W5}, and R^{W6} each independently represent an aliphatic hydrocarbon group which may have a substituent, an aromatic ring group which may have a substituent, or an aliphatic heterocyclic group which may have a substituent, and
* represents a bonding position.

2. The photoelectric conversion element according to claim 1,
wherein X¹ is an oxygen atom.

3. The photoelectric conversion element according to claim 1,
wherein X¹ and X³ are oxygen atoms.

4. The photoelectric conversion element according to claim 1,
wherein the group represented by Formula (A-1) is a group represented by Formula (A-2), 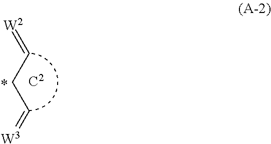
in Formula (A-2), C² represents a ring which contains at least three carbon atoms and may have a substituent,
W² and W³ each independently represent an oxygen atom, a sulfur atom, ═NR^{W1}, or ═CR^{W2}R^{W3}, R^{W1}, R^{W2}, and R^{W3} each have the same meaning as R^{W1}, R^{W2}, and R^{W3} in Formula (1), and
* represents a bonding position.

5. The photoelectric conversion element according to claim 1,
wherein the group represented by Formula (A-1) is a group represented by Formula (C-1) or a group represented by Formula (C-2), 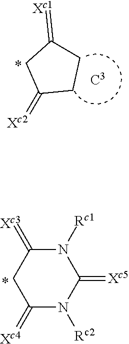
in Formula (C-1), X^{c1} and X^{c2} each independently represent an oxygen atom, a sulfur atom, ═NR^{W1}, or ═CR^{W2}R^{W3}, R^{W1}, R^{W2}, and R^{W3} each have the same meaning as R^{W1}, R^{W2}, and R^{W3} in Formula (1),
C³ represents an aromatic ring which may have a substituent, and
* represents a bonding position,
in Formula (C-2), X^{c3} to X^{c5} each independently represent an oxygen atom, a sulfur atom, ═NR^{W1}, or ═CR^{W2}R^{W3}, R^{W1}, R^{W2}, and R^{W3} each have the same meaning as R^{W1}, R^{W2}, and R^{W3} in Formula (1),
R^{c1} and Rc2 each independently represent a hydrogen atom or a substituent, and
* represents a bonding position.

6. The photoelectric conversion element according to claim 1,
wherein R^{Z2} represents a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, which may have a substituent selected from a substituent group S, an acyl group having 2 to 5 carbon atoms, an aromatic ring group which may have a substituent selected from the substituent group S, an aliphatic heterocyclic group which may have a substituent selected from the substituent group S, or a group represented by —Si(R^{Si2})₃,
provided that the linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms, which may have a substituent selected from the substituent group S, each of which is represented by R^{Z2}, may have a halogen atom and may have an etheric oxygen atom, and the acyl group having 2 to 5 carbon atoms, represented by R^{Z2}, may have a halogen atom,
the substituent group S: a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, a halogen atom, and a group represented by —Si(R^{Si2})₃,
the linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms in the substituent group S may have a halogen atom and may have an etheric oxygen atom,
R^{Si2}'s each independently represent a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, which may have a substituent selected from the substituent group S, or an aromatic ring group which may have a substituent selected from the substituent group S, and
the linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms, which may have a substituent selected from the substituent group S, each of which is represented by R^{Si2}, may have a halogen atom and may have an etheric oxygen atom.

7. The photoelectric conversion element according to claim 1,
wherein R^{Z2} represents a linear aliphatic hydrocarbon group having 1 or 2 carbon atoms, a branched aliphatic hydrocarbon group having 3 or 4 carbon atoms, an acyl group having 2 or 3 carbon atoms, a cyclic aliphatic hydrocarbon group having 3 to 6 carbon atoms, which may have a substituent selected from a substituent group T, an aromatic ring group which may have a substituent selected from the substituent group T, an aliphatic heterocyclic group which may have a substituent selected from the substituent group T, or a group represented by —Si(R^{Si3})₃,
the substituent group T: a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, a halogen atom, and a group represented by —Si(R^{Si3})₃,
R^{Si3}'s each independently represent a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, which may have a substituent selected from the substituent group T, or an aromatic ring group which may have a substituent selected from the substituent group T.

8. The photoelectric conversion element according to claim 1,
wherein the photoelectric conversion film further contains an n-type organic semiconductor, and
the photoelectric conversion film has a bulk hetero structure formed in a state in which the compound represented by Formula (1) and the n-type organic semiconductor are mixed with each other.

9. The photoelectric conversion element according to claim 8,
wherein the n-type organic semiconductor includes fullerenes selected from the group consisting of a fullerene and derivatives of the fullerene.

10. The photoelectric conversion element according to claim 1,
wherein the photoelectric conversion film further contains a p-type organic semiconductor.

11. The photoelectric conversion element according to claim 1,
wherein the photoelectric conversion film further contains a coloring agent.

12. The photoelectric conversion element according to claim 1, further comprising:
one or more interlayers between the conductive film and the transparent conductive film, in addition to the photoelectric conversion film.

13. An imaging element comprising:
the photoelectric conversion element according to claim 1.

14. An optical sensor comprising:
the photoelectric conversion element according to claim 1.

15. A compound represented by Formula (1), 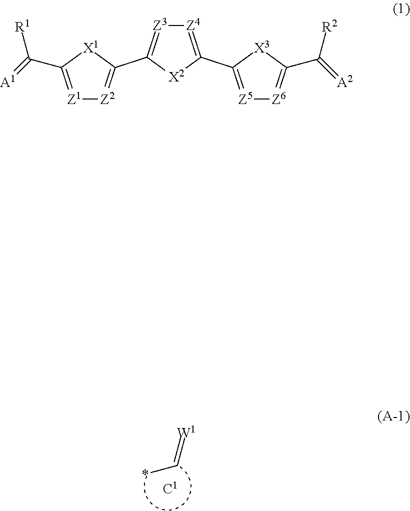
in Formula (1), R¹ and R² each independently represent a hydrogen atom or a substituent,
X¹ to X³ each independently represent a sulfur atom, an oxygen atom, a selenium atom, or a tellurium atom, where at least one of X¹, X², or X³ is an oxygen atom,
Z¹ to Z⁶ each independently represent —CR^{Z1}═ or —N═, R^{Z1} represents a hydrogen atom or a substituent, where at least one of Z¹, . . . , or Z⁶ is —CR^{Z2}═, R^{Z2} represents an aliphatic hydrocarbon group which may have a substituent, an acyl group which may have a substituent, an aromatic ring group which may have a substituent, an aliphatic heterocyclic group which may have a substituent, or a group represented by —Si(R^{Si})₃,
the aliphatic hydrocarbon group represented by R^{Z2} may have a halogen atom and may have an etheric oxygen atom, the acyl group represented by R^{Z2} may have a halogen atom,
R^{Si}'s each independently represent an aliphatic hydrocarbon group which may have a substituent or an aromatic ring group which may have a substituent,
the aliphatic hydrocarbon group represented by R^{Si} may have a halogen atom and may have an etheric oxygen atom,
in a case where Z¹ and Z² are —CR^{Z2}═, two R^{Z2}'s may be bonded to each other to form a ring, in a case where Z³ and Z⁴ are —CR^{Z2}═, two R^{Z2}'s may be bonded to each other to form a ring, and in a case where Z⁵ and Z⁶ are —CR^{Z2}═, two R^{Z2}'s may be bonded to each other to form a ring, and
A¹ and A² each independently represent a group represented by Formula (A-1),
in Formula (A-1), C¹ represents a ring which contains at least two carbon atoms and may have a substituent,
W¹ represents an oxygen atom, a sulfur atom, ═NR^{W1}, or ═CR^{W2}R^{W3}, R^{W1} represents a hydrogen atom or a substituent, R^{W2} and R^{W3} each independently represent a cyano group, —SO₂R^{W4}, —COOR^{W5}, or —COR^{W6}, R^{W4}, R^{W5}, and R^{W6} each independently represent an aliphatic hydrocarbon group which may have a substituent, an aromatic ring group which may have a substituent, or an aliphatic heterocyclic group which may have a substituent, and
* represents a bonding position.

16. The compound according to claim 15,
wherein X¹ is an oxygen atom.

17. The compound according to claim 15,
wherein X¹ and X³ are oxygen atoms.

18. The compound according to claim 15,
wherein the group represented by Formula (A-1) is a group represented by Formula (A-2), 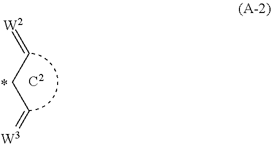
in Formula (A-2), C² represents a ring which contains at least three carbon atoms and may have a substituent,
W² and W³ each independently represent an oxygen atom, a sulfur atom, ═NR^{W1}, or ═CR^{W2}R^{W3}, R^{W1}, R^{W2}, and R^{W3} each have the same meaning as R^{W1}, R^{W2}, and R^{W3} in Formula (1), and
* represents a bonding position.

19. The compound according to claim 15,
wherein the group represented by Formula (A-1) is a group represented by Formula (C-1) or a group represented by Formula (C-2), 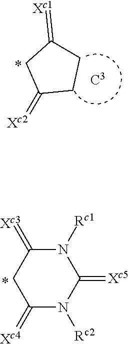
in Formula (C-1), X^{c1} and X^{c2} each independently represent an oxygen atom, a sulfur atom, ═NR^{W1}, or ═CR^{W2}R^{W3}, R^{W1}, R^{W2}, and R^{W3} each have the same meaning as R^{W1}, R^{W2} and R^{W3} in Formula (1),
C³ represents an aromatic ring which may have a substituent, and
* represents a bonding position,
in Formula (C-2), X^{c3} to X^{c5} each independently represent an oxygen atom, a sulfur atom, ═NR^{W1}, or ═CR^{W2}R^{W3}, R^{W1}, R^{W2}, and R^{W3} each have the same meaning as R^{W1}, R^{W2}, and R^{W3} in Formula (1),
R^{c1} and R^{c2} each independently represent a hydrogen atom or a substituent, and
* represents a bonding position.

20. The compound according to claim 1,
wherein R^{Z2} represents a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, which may have a substituent selected from a substituent group S, an acyl group having 2 to 5 carbon atoms, an aromatic ring group which may have a substituent selected from the substituent group S, an aliphatic heterocyclic group which may have a substituent selected from the substituent group S, or a group represented by —Si(R^{Si2})₃,
provided that the linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms, which may have a substituent selected from the substituent group S, each of which is represented by R^{Z2}, may have a halogen atom and may have an etheric oxygen atom, and the acyl group having 2 to 5 carbon atoms, represented by R^{Z2}, may have a halogen atom,
the substituent group S: a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, a halogen atom, and a group represented by —Si(R^{Si2})₃,
the linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms in the substituent group S may have a halogen atom and may have an etheric oxygen atom,
R^{Si2}'s each independently represent a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, which may have a substituent selected from the substituent group S, or an aromatic ring group which may have a substituent selected from the substituent group S, and
the linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms, which may have a substituent selected from the substituent group S, each of which is represented by R^{Si2}, may have a halogen atom and may have an etheric oxygen atom.

21. The compound according to claim 1,
wherein R^{Z2} represents a linear aliphatic hydrocarbon group having 1 or 2 carbon atoms, a branched aliphatic hydrocarbon group having 3 or 4 carbon atoms, an acyl group having 2 or 3 carbon atoms, a cyclic aliphatic hydrocarbon group having 3 to 6 carbon atoms, which may have a substituent selected from a substituent group T, an aromatic ring group which may have a substituent selected from the substituent group T, an aliphatic heterocyclic group which may have a substituent selected from the substituent group T, or a group represented by —Si(R^{Si3})₃,
the substituent group T: a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, a halogen atom, and a group represented by —Si(R^{Si3})₃,
R^{Si3}'s each independently represent a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, which may have a substituent selected from the substituent group T, or an aromatic ring group which may have a substituent selected from the substituent group T.
